(19) United States
(12) Patent Application Publication
(10) Pub. No.: US 2009/0048291 A1
(43) Pub. Date: Feb. 19, 2009

(54) Novel Piperidine-4-Acetic Acid Derivatives and Their Use

(75) Inventors: Jacques Huck, Lyon (FR); Frederic Ooms, Hannut (BE); Julien Parcq, Valenciennes (FR); Regereau Yannick, Bruxellese (BE); Hamid R. Hoveyda, Bruxelles (BE); Guillaume Dutheuil, Flenu (BE)

Correspondence Address: EDWARDS ANGELL PALMER & DODGE LLP, P.O. BOX 55874, BOSTON, MA, 02205, US

(73) Assignee: Euroscreen S.A., Gosselies (BE)

(21) Appl. No.: 12/185,475

(22) Filed: Aug. 4, 2008

Related U.S. Application Data
(63) Continuation of application No. PCT/EP2008/059136, filed on Jul. 11, 2008.
(60) Provisional application No. 60/959,331, filed on Jul. 13, 2007.

(30) Foreign Application Priority Data
Jul. 13, 2007 (EP) 07112470.5

(51) Int. Cl. A61K 31/439; C07D 211/34; A61K 31/445; A61K 31/4545; A61P 31/18; A61P 29/00; C07D 401/12; C07D 453/02

(52) U.S. Cl. 514/305; 546/234; 514/331; 514/318; 546/194; 546/133

(57) ABSTRACT

The present invention is directed to compounds of formula I

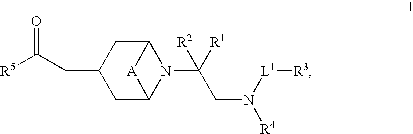

compositions comprising them and their use.

The present invention relates to pharmaceutically active piperidine derivatives and their use as agonists of CC chemokine receptor activity, more specifically of CCR5 activity. Chemokines are chemotactic cytokines which play an important role in immune and inflammatory responses.

### BACKGROUND OF THE INVENTION

The Chemokines comprise a large family of proteins which have common important structural features and which have the ability to attack leukocytes. The chemokine family is divided into two main groups exhibiting characteristic structural motifs, the Cys-X-Cys (CXC) and Cys-Cys (CC) subfamilies.

CC chemokine receptors are integral membrane proteins that specifically bind and respond to cytokines of the CC chemokine family. They represent one subfamily of chemokine receptors, a large family of G protein-linked receptors that are known as seven transmembrane (7-TM) proteins since they span the cell membrane seven times. To date, ten true members of the CC chemokine receptor subfamily have been described. These are named CCR1 to CCR10 according to the IUIS/WHO Subcommittee on Chemokine Nomenclature.

Among the CC chemokine receptors, CCR5 is defined as a major co-receptor implicated in susceptibility to HIV-1 infection and disease. CCR5 is

a receptor expressed on several cell types including T-lymphocytes, peripheral blood-derived dendritic cells, CD34+ hematopoietic progenitor cells and certain activated/memory Th1 lymphocytes.

Because of this well-known activity as HIV-1 co-receptor, antagonists for CCR5 have been developed with the aim of inhibiting CCR5-mediated HIV entry. The most advanced of these, Maraviroc, from Pfizer, is in good way to obtain the final FDA approval for entry on the market.

In the prior art, such as in WO0276948, for example, blocking this receptor with a CCR5 antagonist or inducing receptor internalization with a CCR5 agonist was considered of great interest to protect cells from viral infection by HIV-1.

WO0276948 describe compounds having activity as pharmaceuticals, in particular as modulators (such as agonists, partial agonists, inverse agonists or antagonists) of chemokine receptor (especially CCR5) activity.

This invention proposes alternative compounds having activity as pharmaceuticals, in particular as modulators (such as agonists, partial agonists, inverse agonists or antagonists) of chemokine receptor (especially CCR5) for use in the treatment of autoimmune, inflammatory, infectious, proliferative, hyperproliferative diseases, or immunologically-mediated diseases (including rejection of transplanted organs or tissues and Acquired Immunodeficiency Syndrome (AIDS)).

### SUMMARY OF THE INVENTION

The invention encompasses compounds of general formula (I) and methods of use of such compounds or compositions as chemokine receptor modulators.

In a general aspect, the invention provides compounds of general formula I:

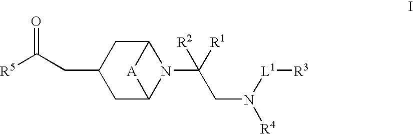

and pharmaceutically acceptable salts and solvates thereof, wherein
A is —CH₂—CH₂— or absent;
R¹ and R² independently are H, halo, optionally substituted alkyl, aryl, heteroaryl, cycloalkyl, cycloalkylalkyl, heterocyclyl;
R³ and R⁴ independently are a group selected from aryl, heteroaryl, cycloalkyl, and heterocyclyl, each group being optionally substituted by one or more substituent(s) selected from halo, oxo, nitro, cyano, azido, alkyl, hydroxyalkyl, haloalkyl, cycloalkyl, cycloalkylalkyl, alkenyl, alkynyl, heteroalkyl, heterocyclyl, heterocyclylalkyl, aryl, aralkyl, heteroaryl, heteroarylalkyl, hydroxyl, alkoxy, haloalkoxy, cycloalkyloxy, heterocyclyloxy, aryloxy, thiol, alkylthio, thioalkyl, haloalkylthio, acyl, thioacyl, aroyl, amino, alkylamino, aminoalkyl, carboxy, alkoxycarbonyl, cycloalkyloxycarbonyl, heterocyclyloxycarbonyl, aryloxycarbonyl, heteroaryloxycarbonyl, alkylcarbonyloxy, cycloalkylcarbonyloxy, heterocyclylcarbonyloxy, arylcarbonyloxy, heteroarylcarbonyloxy, arylalkyloxy, alkylcarbonylamino, haloalkylcarbonylamino, cycloalkylcarbonylamino, heterocyclylcarbonylamino arylcarbonylamino, heteroarylcarbonylamino, alkylcarbonylaminoalkyl, acylamino, carbamoyl, hydroxycarbamoyl, alkylcarbamoyl, arylcarbamoyl, heteroarylcarbamoyl, carbamoylalkyl, carbamoylamino, alkylcarbamoylamino, sulfino, alkylsulfinyl, sulfo, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, haloalkylsulfonylamino, or two substituents form an alkylenedioxy group or a haloalkylenedioxy group, or fused to the cycloalkyl, aryl, or heterocyclyl group may be one or more cycloalkyl, aryl, heterocyclyl or heteroaryl group, each of said group being optionally substituted by one or more further substituent(s) selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, nitro, oxo, and sulfonyl; more preferably, R³ and R⁴ independently are a group selected from aryl, heteroaryl, cycloalkyl, and heterocyclyl, such as phenyl, pyridinyl, and cyclohexyl, each group being optionally substituted by one or more substituent(s) selected from halo, cyano, SO2R, or SO2NR′R″ wherein R is an alkyl and R′, R″ are H or alkyl.

L¹ is NRCO, NRSO₂, CO, CONR, CONRCH₂, CH₂CO, COCH₂ CH₂CH₂CO, CH₂COCH₂, COCH₂CH₂, SO₂, SO₂NR, SO₂CH₂, SO₂CH₂CH₂, a single bond or a group selected from C₁-C₃ alkylene, C₂-C₄ alkenylene and C₂-C₄ alkynylene, each group being optionally substituted with one or more substituent(s) selected from alkyl, aryl, heteroaryl, halo, alkylcarbonyl, alkylamino, alkoxy, alkylcarbonylamino, and alkylcarbonylalkyl, wherein R is hydrogen or C₁-C₆ alkyl;

R⁵ is selected from NR⁶(L²-R⁸), O (L²-R⁸), and CR⁶R⁷ (L²-R⁸);
R⁶ and R⁷ independently are selected from hydrogen, C₁-C₄ alkyl, allyl, propargyl, —CH₂—CH₂—OH, —CH₂—CH₂—CH₂—OH, cyclopropyl, cyclopropylmethyl, aryl, and heteroaryl;
L² is a single bond or C₁-C₄ alkylene, optionally substituted by one or more substituent(s) selected from halo, oxo, cyano, alkyl, hydroxyalkyl, haloalkyl, cycloalkyl, cycloalkylalkyl, and alkoxy, or L² is CR^{a}R^{b}, wherein R^{a} and R^{b} form together with the carbon to which they are attached a carbocycle having 3 to 6 ring atoms;
R⁸ is a group selected from aryl, heteroaryl, cycloalkyl, and heterocyclyl, each group being optionally substituted by one or more substituent(s) selected from halo, oxo, nitro, cyano, azido, alkyl, hydroxyalkyl, haloalkyl, cycloalkyl, cycloalkylalkyl, alkenyl, alkynyl, heteroalkyl, heterocyclyl, heterocyclylalkyl, aryl, aralkyl, heteroaryl, heteroarylalkyl, hydroxyl, alkoxy, haloalkoxy, cycloalkyloxy, heterocyclyloxy, aryloxy, thiol, alkylthio, thioalkyl, haloalkylthio, acyl, thioacyl, aroyl, amino, alkylamino, aminoalkyl, carboxy, alkoxycarbonyl, cycloalkyloxycarbonyl, heterocyclyloxycarbonyl, aryloxycarbonyl, heteroaryloxycarbonyl, alkylcarbonyloxy, cycloalkylcarbonyloxy, heterocyclylcarbonyloxy, arylcarbonyloxy, heteroarylcarbonyloxy, arylalkyloxy, alkylcarbonylamino, haloalkylcarbonylamino, cycloalkylcarbonylamino, heterocyclylcarbonylamino arylcarbonylamino, heteroarylcarbonylamino, alkylcarbonylaminoalkyl, acylamino, carbamoyl, hydroxycarbamoyl, alkylcarbamoyl, arylcarbamoyl, heteroarylcarbamoyl, carbamoylalkyl, carbamoylamino, alkylcarbamoylamino, sulfino, alkylsulfinyl, sulfo, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, and haloalkylsulfonylamino, or two substituents form an alkylenedioxy group or a haloalkylenedioxy group, or fused to the cycloalkyl, aryl, or heterocyclyl group may be one or more cycloalkyl, aryl, heterocyclyl or heteroaryl group, each of said group being optionally substituted by one or more further substituent(s) selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, nitro, oxo, and sulfonyl, or
R⁶ and L²-R⁸ form together with the nitrogen atom to which they are connected a 5 to 8 membered saturated, or unsaturated cycle, which cycle is optionally substituted by one or more groups selected from aryl, heteroaryl, cycloalkyl, and heterocyclyl, each group being optionally substituted by one or more substituent(s) selected from halo, oxo, nitro, cyano, azido, alkyl, hydroxyalkyl, haloalkyl, cycloalkyl, cycloalkylalkyl, alkenyl, alkynyl, heteroalkyl, heterocyclyl, heterocyclylalkyl, aryl, aralkyl, heteroaryl, heteroarylalkyl, hydroxyl, alkoxy, haloalkoxy, cycloalkyloxy, heterocyclyloxy, aryloxy, thiol, alkylthio, thioalkyl, haloalkylthio, acyl, thioacyl, aroyl, amino, alkylamino, aminoalkyl, carboxy, alkoxycarbonyl, cycloalkyloxycarbonyl, heterocyclyloxycarbonyl, aryloxycarbonyl, heteroaryloxycarbonyl, alkylcarbonyloxy, cycloalkylcarbonyloxy, heterocyclylcarbonyloxy, arylcarbonyloxy, heteroarylcarbonyloxy, arylalkyloxy, alkylcarbonylamino, haloalkylcarbonylamino, cycloalkylcarbonylamino, heterocyclylcarbonylamino arylcarbonylamino, heteroarylcarbonylamino, alkylcarbonylaminoalkyl, acylamino, carbamoyl, hydroxycarbamoyl, alkylcarbamoyl, arylcarbamoyl, heteroarylcarbamoyl, carbamoylalkyl, carbamoylamino, alkylcarbamoylamino, sulfino, alkylsulfinyl, sulfo, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, and haloalkylsulfonylamino, or two substituents form an alkylenedioxy group or a haloalkylenedioxy group, or fused to the 5 to 8 membered saturated, or unsaturated cycle may be one or more cycloalkyl, aryl, heterocyclyl or heteroaryl group, each of said groups being optionally substituted by one or more further substituent(s) selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, nitro, oxo, and sulfonyl, or
R⁶ and L²-R⁸ form together with the carbon atom to which they are connected a 5 to 8 membered saturated, partially unsaturated or aromatic cycle, which cycle is optionally substituted by one or more groups selected from aryl, heteroaryl, cycloalkyl, and heterocyclyl, each group being optionally substituted by one or more substituent(s) selected from halo, oxo, nitro, cyano, azido, alkyl, hydroxyalkyl, haloalkyl, cycloalkyl, cycloalkylalkyl, alkenyl, alkynyl, heteroalkyl, heterocyclyl, heterocyclylalkyl, aryl, aralkyl, heteroaryl, heteroarylalkyl, hydroxyl, alkoxy, haloalkoxy, cycloalkyloxy, heterocyclyloxy, aryloxy, thiol, alkylthio, thioalkyl, haloalkylthio, acyl, thioacyl, aroyl, amino, alkylamino, aminoalkyl, carboxy, alkoxycarbonyl, cycloalkyloxycarbonyl, heterocyclyloxycarbonyl, aryloxycarbonyl, heteroaryloxycarbonyl, alkylcarbonyloxy, cycloalkylcarbonyloxy, heterocyclylcarbonyloxy, arylcarbonyloxy, heteroarylcarbonyloxy, arylalkyloxy, alkylcarbonylamino, haloalkylcarbonylamino, cycloalkylcarbonylamino, heterocyclylcarbonylamino arylcarbonylamino, heteroarylcarbonylamino, alkylcarbonylaminoalkyl, acylamino, carbamoyl, hydroxycarbamoyl, alkylcarbamoyl, arylcarbamoyl, heteroarylcarbamoyl, carbamoylalkyl, carbamoylamino, alkylcarbamoylamino, sulfino, alkylsulfinyl, sulfo, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, and haloalkylsulfonylamino, or two substituents form an alkylenedioxy group or a haloalkylenedioxy group, or fused to the 5 to 8 membered saturated, partially unsaturated or aromatic cycle may be one or more cycloalkyl, aryl, heterocyclyl or heteroaryl group, each of said groups being optionally substituted by one or more further substituent(s) selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, nitro, oxo, and sulfonyl.

The invention also relates to the use of the above compounds or their pharmaceutically acceptable salts and solvates as modulators of CCR5, preferably as antagonists or agonists of CCR5, and even more preferably as agonists of CCR5.

The invention further provides methods for the treatment or prevention of autoimmune, inflammatory, infectious, proliferative, hyperproliferative diseases, or immunologically-mediated diseases (including rejection of transplanted organs or tissues and Acquired Immunodeficiency Syndrome (AIDS)).

### DETAILED DESCRIPTION OF THE INVENTION

As noted above, the invention relates to compounds of formula I, as well as their pharmaceutically acceptable salts and solvates.

Preferred compounds of formula I and pharmaceutically acceptable salts and solvates thereof are those wherein

A is absent;
R¹ and R² independently are hydrogen, or C₁-C₄ alkyl; preferably hydrogen or methyl;
L¹, R³, R⁴ and R⁸ are as defined above in respect of general formula I;

### R⁵ is NR⁶ (L²-R⁸)

R⁶ is selected from hydrogen, C₁-C₄ alkyl, allyl, propargyl, —CH₂—CH₂—OH, —CH₂—CH₂—CH₂—OH, cyclopropyl, cyclopropylmethyl, aryl, and heteroaryl; preferably hydrogen or C₁-C₄ alkyl; most preferably hydrogen; and
L² is a single bond.

Even more preferred compounds of formula I and pharmaceutically acceptable salts and solvates thereof are those wherein

R³ is as defined above in respect of general formula I;
A is absent;
R¹ is hydrogen;
R² is hydrogen or methyl, preferably methyl;
R⁴ is aryl, optionally substituted by one or more substituent(s) selected from halo, oxo, nitro, cyano, azido, alkyl, hydroxyalkyl, haloalkyl, cycloalkyl, cycloalkylalkyl, alkenyl, alkynyl, heteroalkyl, heterocyclyl, heterocyclylalkyl, aryl, aralkyl, heteroaryl, heteroarylalkyl, hydroxyl, alkoxy, haloalkoxy, cycloalkyloxy, heterocyclyloxy, aryloxy, thiol, alkylthio, thioalkyl, haloalkylthio, acyl, thioacyl, aroyl, amino, alkylamino, aminoalkyl, carboxy, alkoxycarbonyl, cycloalkyloxycarbonyl, heterocyclyloxycarbonyl, aryloxycarbonyl, heteroaryloxycarbonyl, alkylcarbonyloxy, cycloalkylcarbonyloxy, heterocyclylcarbonyloxy, arylcarbonyloxy, heteroarylcarbonyloxy, arylalkyloxy, alkylcarbonylamino, haloalkylcarbonylamino, cycloalkylcarbonylamino, heterocyclylcarbonylamino arylcarbonylamino, heteroarylcarbonylamino, alkylcarbonylaminoalkyl, acylamino, carbamoyl, hydroxycarbamoyl, alkylcarbamoyl, arylcarbamoyl, heteroarylcarbamoyl, carbamoylalkyl, carbamoylamino, alkylcarbamoylamino, sulfino, alkylsulfinyl, sulfo, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, and haloalkylsulfonylamino, or two substituents form an alkylenedioxy group or a haloalkylenedioxy group, or fused to the aryl group may be one or more cycloalkyl, aryl, heterocyclyl or heteroaryl group, each of said group being optionally substituted by one or more further substituent(s) selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, nitro, oxo, and sulfonyl; more preferably, R⁴ is aryl optionally substituted by one or more substituent(s) selected from halo, cyano, SO2R, or SO₂NR′R″ wherein R is an alkyl and R′, R″ are H or alkyl;
L¹ is as defined above in respect of general formula I;

### R⁵ is NR⁶(L²-R⁸);

R⁶ is hydrogen;
L² is a single bond; and
R⁸ is as defined above in respect of general formula I.

In one embodiment, preferred compounds of formula I are those of formula Ia:

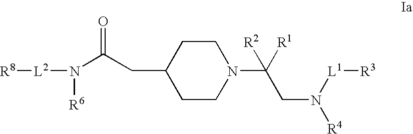

and pharmaceutically acceptable salts and solvates thereof, wherein
R¹, R², R³, and R⁴ are as defined above in respect of general formula I,
L¹ is as defined above in respect of Formula I, preferably L¹ is CO, CONH, CONHCH₂, CH₂CO, COCH₂ CH₂CH₂CO, CH₂COCH₂, COCH₂CH₂, SO₂, SO₂NH, SO₂CH₂, SO₂CH₂CH₂, a single bond or a group selected from C₁-C₃ alkylene, C₂-C₄ alkenylene, and C₂-C₄ alkylylene, each group being optionally substituted with one or more substituent(s) selected from alkyl, aryl, heteroaryl, halo, alkylcarbonyl, alkylamino, alkoxy, alkylcarbonylamino, and alkylcarbonylalkyl; and
R⁶, L² and R⁸ are as defined above in respect of Formula I.

Preferred compounds of formula Ia are those wherein

L¹, R³, R⁴ and R⁸ are as defined above in respect of general formula Ia;
R¹ and R² independently are hydrogen, or C₁-C₄ alkyl; preferably hydrogen or methyl;
R⁶ is selected from hydrogen, C₁-C₄ alkyl, allyl, propargyl, —CH₂—CH₂—OH, —CH₂—CH₂—CH₂—OH, cyclopropyl, cyclopropylmethyl, aryl, and heteroaryl; preferably hydrogen or C₁-C₄ alkyl; most preferably hydrogen; and
L² is a single bond.

In another embodiment, preferred compounds of formula I are those of formula Ib:

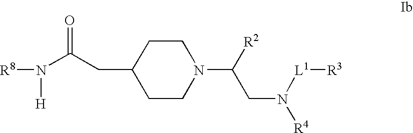

and pharmaceutically acceptable salts and solvates thereof, wherein
R² is H, or C₁-C₄ alkyl, preferably methyl;
R³ and R⁴ are as defined above in respect of formula I;
L¹ is CO, CONH, CONHCH₂, CH₂CO, COCH₂ CH₂CH₂CO, CH₂COCH₂, COCH₂CH₂, SO₂NH, SO₂, SO₂CH₂, SO₂CH₂CH₂, a single bond or a group selected from C₁-C₃ alkylene, C₂-C₄ alkenylene and C₂-C₄ alkynylene, each group being optionally substituted with one or more substituent(s) selected from alkyl, aryl, heteroaryl, halo, alkylcarbonyl, alkylamino, alkoxy, alkylcarbonylamino, and alkylcarbonylalkyl; and
R⁸ is a group selected from aryl, heteroaryl, cycloalkyl, and heterocyclyl, each group being optionally substituted by one or more substituent(s) selected from halo, oxo, nitro, cyano, azido, alkyl, hydroxyalkyl, haloalkyl, cycloalkyl, cycloalkylalkyl, alkenyl, alkynyl, heteroalkyl, heterocyclyl, heterocyclylalkyl, aryl, aralkyl, heteroaryl, heteroarylalkyl, hydroxyl, alkoxy, haloalkoxy, cycloalkyloxy, heterocyclyloxy, aryloxy, thiol, alkylthio, thioalkyl, haloalkylthio, acyl, thioacyl, aroyl, amino, alkylamino, aminoalkyl, carboxy, alkoxycarbonyl, cycloalkyloxycarbonyl, heterocyclyloxycarbonyl, aryloxycarbonyl, heteroaryloxycarbonyl, alkylcarbonyloxy, cycloalkylcarbonyloxy, heterocyclylcarbonyloxy, arylcarbonyloxy, heteroarylcarbonyloxy, arylalkyloxy, alkylcarbonylamino, haloalkylcarbonylamino, cycloalkylcarbonylamino, heterocyclylcarbonylamino arylcarbonylamino, heteroarylcarbonylamino, alkylcarbonylaminoalkyl, acylamino, carbamoyl, hydroxycarbamoyl, alkylcarbamoyl, arylcarbamoyl, heteroarylcarbamoyl, carbamoylalkyl, carbamoylamino, alkylcarbamoylamino, sulfino, alkylsulfinyl, sulfo, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, and haloalkylsulfonylamino, or two substituents form an alkylenedioxy group or a haloalkylenedioxy group, or fused to the cycloalkyl, aryl, or heterocyclyl group may be one or more cycloalkyl, aryl, heterocyclyl or heteroaryl group, each of said groups being optionally substituted by one or more further substituent(s) selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, nitro, oxo, and sulfonyl.

Preferred compounds of formula Ib are those wherein

R³, R⁸ and L¹ are as defined above in respect of general formula Ib;

R² is hydrogen, or C₁-C₄ alkyl; preferably hydrogen or methyl; most preferably methyl; and

R⁴ is aryl, preferably phenyl, optionally substituted by one or more substituent(s) selected from halo, oxo, nitro, cyano, azido, alkyl, hydroxyalkyl, haloalkyl, cycloalkyl, cycloalkylalkyl, alkenyl, alkynyl, heteroalkyl, heterocyclyl, heterocyclylalkyl, aryl, aralkyl, heteroaryl, heteroarylalkyl, hydroxyl, alkoxy, haloalkoxy, cycloalkyloxy, heterocyclyloxy, aryloxy, thiol, alkylthio, thioalkyl, haloalkylthio, acyl, thioacyl, aroyl, amino, alkylamino, aminoalkyl, carboxy, alkoxycarbonyl, cycloalkyloxycarbonyl, heterocyclyloxycarbonyl, aryloxycarbonyl, heteroaryloxycarbonyl, alkylcarbonyloxy, cycloalkylcarbonyloxy, heterocyclylcarbonyloxy, arylcarbonyloxy, heteroarylcarbonyloxy, arylalkyloxy, alkylcarbonylamino, haloalkylcarbonylamino, cycloalkylcarbonylamino, heterocyclylcarbonylamino arylcarbonylamino, heteroarylcarbonylamino, alkylcarbonylaminoalkyl, acylamino, carbamoyl, hydroxycarbamoyl, alkylcarbamoyl, arylcarbamoyl, heteroarylcarbamoyl, carbamoylalkyl, carbamoylamino, alkylcarbamoylamino, sulfino, alkylsulfinyl, sulfo, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, and haloalkylsulfonylamino, or two substituents form an alkylenedioxy group or a haloalkylenedioxy group, or fused to the aryl, preferably phenyl, group may be one or more cycloalkyl, aryl, heterocyclyl or heteroaryl group, each of said substituents being optionally substituted by one or more further substituent(s) selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, nitro, oxo, and sulfonyl; more preferably, R⁴ is aryl, preferably phenyl, optionally substituted by one or more substituent(s) selected from halo, cyano, SO2R, or SO2NR′R″ wherein R is an alkyl and R′, R″ are H or alkyl.

Most preferred compounds of formula Ib are those wherein

R² is H, or C₁-C₄ alkyl, preferably methyl;

R³ is as defined above in respect of general Formula Ib;

R⁴ and R⁸ independently are aryl, preferably phenyl, optionally substituted by one or more substituent(s) selected from halo, oxo, nitro, cyano, azido, alkyl, hydroxyalkyl, haloalkyl, cycloalkyl, cycloalkylalkyl, alkenyl, alkynyl, heteroalkyl, heterocyclyl, heterocyclylalkyl, aryl, aralkyl, heteroaryl, heteroarylalkyl, hydroxyl, alkoxy, haloalkoxy, cycloalkyloxy; heterocyclyloxy, aryloxy, thiol, alkylthio, thioalkyl, haloalkylthio, acyl, thioacyl, aroyl, amino, alkylamino, aminoalkyl, carboxy, alkoxycarbonyl, cycloalkyloxycarbonyl, heterocyclyloxycarbonyl, aryloxycarbonyl, heteroaryloxycarbonyl, alkylcarbonyloxy, cycloalkylcarbonyloxy, heterocyclylcarbonyloxy, arylcarbonyloxy, heteroarylcarbonyloxy, arylalkyloxy, alkylcarbonylamino, haloalkylcarbonylamino, cycloalkylcarbonylamino, heterocyclylcarbonylamino arylcarbonylamino, heteroarylcarbonylamino, alkylcarbonylaminoalkyl, acylamino, carbamoyl, hydroxycarbamoyl, alkylcarbamoyl, arylcarbamoyl, heteroarylcarbamoyl, carbamoylalkyl, carbamoylamino, alkylcarbamoylamino, sulfino, alkylsulfinyl, sulfo, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, and haloalkylsulfonylamino, or two substituents form an alkylenedioxy group or a haloalkylenedioxy group, or fused to the aryl, preferably phenyl, group may be one or more cycloalkyl, aryl, heterocyclyl or heteroaryl group, each of said groups being optionally substituted by one or more further substituent(s) selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, nitro, oxo, and sulfonyl; more preferably, R⁴ and R⁸ independently are aryl, preferably phenyl, optionally substituted by one or more substituent(s) selected from halo, cyano, SO2R, or SO2NR′R″ wherein R is an alkyl and R′, R″ are H or alkyl; and

L¹ is CO, CONH, CONHCH₂, CH₂CO, COCH₂ CH₂CH₂CO, CH₂COCH₂, COCH₂CH₂, SO₂NH, SO₂, SO₂CH₂, SO₂CH₂CH₂, a single bond or a group selected from C₁-C₃ alkylene, C₂-C₄ alkenylene and C₂-C₄ alkynylene, each group being optionally substituted with one or more substituent(s) selected from alkyl, aryl, heteroaryl, halo, alkylcarbonyl, alkylamino, alkoxy, alkylcarbonylamino, and alkylcarbonylalkyl.

In still another embodiment, preferred compounds of formula I are those of formula Ic:

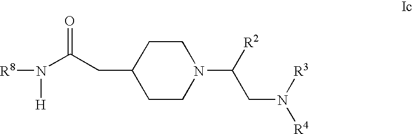

and pharmaceutically acceptable salts and solvates thereof, wherein
R² is H, or C₁-C₄ alkyl, preferably methyl; and
R³, R⁴ and R⁸ independently are aryl, preferably phenyl, optionally substituted by one or more substituent(s) selected from halo, oxo, nitro, cyano, azido, alkyl, hydroxyalkyl, haloalkyl, cycloalkyl, cycloalkylalkyl, alkenyl, alkynyl, heteroalkyl, heterocyclyl, heterocyclylalkyl, aryl, aralkyl, heteroaryl, heteroarylalkyl, hydroxyl, alkoxy, haloalkoxy, cycloalkyloxy, heterocyclyloxy, aryloxy, thiol, alkylthio, thioalkyl, haloalkylthio, acyl, thioacyl, aroyl, amino, alkylamino, aminoalkyl, carboxy, alkoxycarbonyl, cycloalkyloxycarbonyl, heterocyclyloxycarbonyl, aryloxycarbonyl, heteroaryloxycarbonyl, alkylcarbonyloxy, cycloalkylcarbonyloxy, heterocyclylcarbonyloxy, arylcarbonyloxy, heteroarylcarbonyloxy, arylalkyloxy, alkylcarbonylamino, haloalkylcarbonylamino, cycloalkylcarbonylamino, heterocyclylcarbonylamino arylcarbonylamino, heteroarylcarbonylamino, alkylcarbonylaminoalkyl, acylamino, carbamoyl, hydroxycarbamoyl, alkylcarbamoyl, arylcarbamoyl, heteroarylcarbamoyl, carbamoylalkyl, carbamoylamino, alkylcarbamoylamino, sulfino, alkylsulfinyl, sulfo, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, and haloalkylsulfonylamino, or two substituents form an alkylenedioxy group or a haloalkylenedioxy group, or fused to the aryl, preferably phenyl, group may be one or more cycloalkyl, aryl, heterocyclyl or heteroaryl group, each of said groups being optionally substituted by one or more further substituent(s) selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, nitro, oxo, and sulfonyl; more preferably, R³, R⁴ and R⁸ independently are aryl, preferably phenyl optionally substituted by one or more substituent(s) selected from halo, cyano, SO₂R, or SO₂NR′R″ wherein R is an alkyl and R′, R″ are H or alkyl.

In another embodiment, preferred compounds of formula I are those of formula Id:

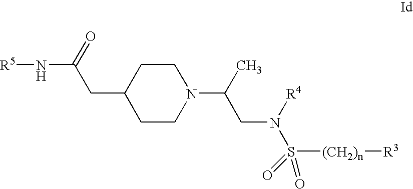

and pharmaceutically acceptable salts and solvates thereof, wherein
n is 0, 1 or 2;
R³ is aryl, heteroaryl or cycloalkyl, preferably phenyl, pyridinyl, or cyclohexyl, optionally substituted by one or more substituent(s) selected from halo, oxo, nitro, cyano, azido, alkyl, hydroxyalkyl, cycloalkyl, alkynyl, hydroxyl, alkoxy, haloalkoxy, thiol, alkylthio, thioalkyl, haloalkylthio, acyl, thioacyl, amino, alkylamino, aminoalkyl, carboxy, alkoxycarbonyl, cycloalkyloxycarbonyl, alkylcarbonyloxy, alkylcarbonylamino, haloalkylcarbonylamino, cycloalkylcarbonylamino, alkylcarbonylaminoalkyl, acylamino, carbamoyl, hydroxycarbamoyl, alkylcarbamoyl, carbamoylalkyl, carbamoylamino, alkylcarbamoylamino, sulfino, alkylsulfinyl, sulfo, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, and haloalkylsulfonylamino, or two substituents form an alkylenedioxy group or a haloalkylenedioxy group, or fused to the phenyl or pyridinyl group may be one or more cycloalkyl, aryl, heterocyclyl or heteroaryl group; more preferably, R³ is phenyl, pyridinyl, or cyclohexyl, each being optionally substituted by one or more substituent(s) selected from halo, cyano, SO₂R, or SO₂NR′R″ wherein R is an alkyl and R′, R″ are H or alkyl;
R⁴ is defined as above in respect of formula I, preferably is a group selected from phenyl, pyridinyl, pyrrolyl, pyrazolyl, imidazolyl, oxazolyl, isooxazolyl, thiazolyl, isothiozalyl, piperidyl, piperazyl, pyrrolidyl, tetrahydropyranyl, tetrahydrofuranyl, tetrahydrothiopyranyl, tetrahydrothiopyranyl-1,1-dioxide, tetrahydrothiophenyl, furanyl, pyrrolyl, thiophenyl, cyclopentyl, cyclohexyl, and indolyl, optionally substituted by one or more substituent(s) selected from halo, oxo, nitro, cyano, azido, alkyl, hydroxyalkyl, cycloalkyl, alkynyl, hydroxyl, alkoxy, haloalkoxy, thiol, alkylthio, thioalkyl, haloalkylthio, acyl, thioacyl, amino, alkylamino, aminoalkyl, carboxy, alkoxycarbonyl, cycloalkyloxycarbonyl, alkylcarbonyloxy, alkylcarbonylamino, haloalkylcarbonylamino, cycloalkylcarbonylamino, alkylcarbonylaminoalkyl, acylamino, carbamoyl, hydroxycarbamoyl, alkylcarbamoyl, carbamoylalkyl, carbamoylamino, alkylcarbamoylamino, sulfino, alkylsulfinyl, sulfo, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, and haloalkylsulfonylamino, or two substituents form an alkylenedioxy group or a haloalkylenedioxy group, or fused to the phenyl, pyridinyl, pyrrolyl, pyrazolyl, imidazolyl, oxazolyl, isooxazolyl, thiazolyl, isothiozalyl, piperidyl, piperazyl, pyrrolidyl, tetrahydropyranyl, tetrahydrofuranyl, tetrahydrothiopyranyl, tetrahydrothiopyranyl-1,1-dioxide, tetrahydrothiophenyl, furanyl, pyrrolyl, thiophenyl, cyclopentyl, cyclohexyl, or indolyl group may be one or more cycloalkyl, aryl, heterocyclyl or heteroaryl group; more preferably, R⁴ is a group selected from phenyl, pyridinyl, pyrrolyl, pyrazolyl, imidazolyl, oxazolyl, isooxazolyl, thiazolyl, isothiozalyl, piperidyl, piperazyl, pyrrolidyl, tetrahydropyranyl, tetrahydrofuranyl, tetrahydrothiopyranyl, tetrahydrothiopyranyl-1,1-dioxide, tetrahydrothiophenyl, furanyl, pyrrolyl, thiophenyl, cyclopentyl, cyclohexyl, and indolyl, and most preferably R⁴ is phenyl, each group being optionally substituted by one or more substituent(s) selected from halo, cyano, HN═C(NH₂)—, SO2R, or SO2NR′R″ wherein R is alkyl and R′, R″ are H or alkyl; and
R⁵ is defined as above in respect of formula (I), preferably R⁵ is a group selected from aryl, heteroaryl, cycloalkyl, and heterocyclyl, each group being optionally substituted by one or more substituent(s) selected from halo, oxo, nitro, cyano, azido, alkyl, hydroxyalkyl, cycloalkyl, alkynyl, hydroxyl, alkoxy, haloalkoxy, thiol, alkylthio, thioalkyl, haloalkylthio, acyl, thioacyl, amino, alkylamino, aminoalkyl, carboxy, alkoxycarbonyl, cycloalkyloxycarbonyl, alkylcarbonyloxy, alkylcarbonylamino, haloalkylcarbonylamino, cycloalkylcarbonylamino, alkylcarbonylaminoalkyl, acylamino, carbamoyl, hydroxycarbamoyl, alkylcarbamoyl, carbamoylalkyl, carbamoylamino, alkylcarbamoylamino, HN═C(NH₂)—, sulfino, alkylsulfinyl, sulfo, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, and haloalkylsulfonylamino, or two substituents form an alkylenedioxy group or a haloalkylenedioxy group, or fused to the cycloalkyl, aryl, heterocyclyl group may be one or more cycloalkyl, aryl, heterocyclyl or heteroaryl group; more preferably, R⁵ is a group selected from aryl, heteroaryl, cycloalkyl, and heterocyclyl, each group being optionally substituted by halo, cyano, HN═C(NH₂)—, SO₂R, or SO₂NR′R″ wherein R is an alkyl and R′, R″ are H or alkyl.

Preferably, compounds of formula Id are those of formula Id′

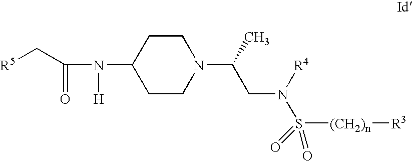

and pharmaceutically acceptable salts and solvates thereof, wherein n, R³, R⁴, and R⁵ are defined as in respect of formula Id above.

Particularly preferred compounds of the invention are those listed in Table 1 hereafter:

TABLE 1

| Compound | Structure |
|---|---|
| 1 | 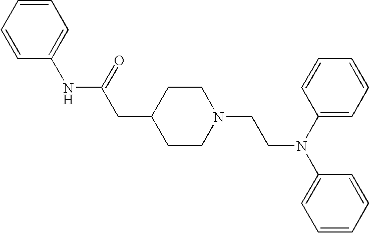 |
| 2 | 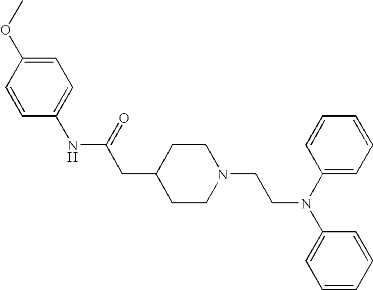 |
| 3 | 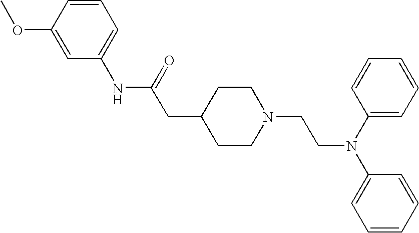 |
| 4 | 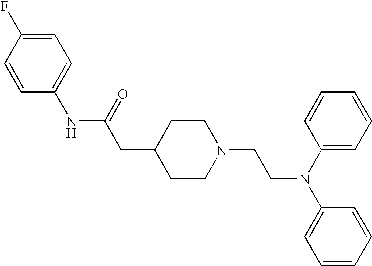 |
| 5 | 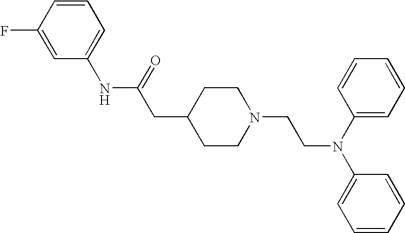 |
| 6 | 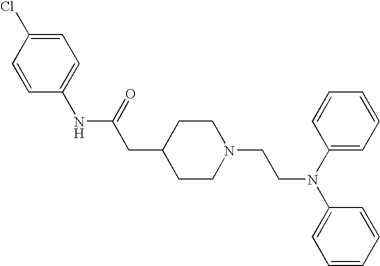 |
| 7 | 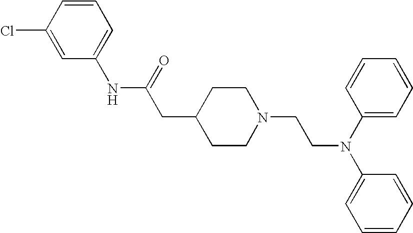 |
| 8 | 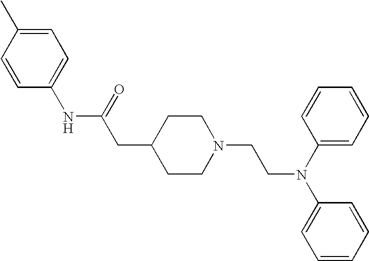 |
| 9 | 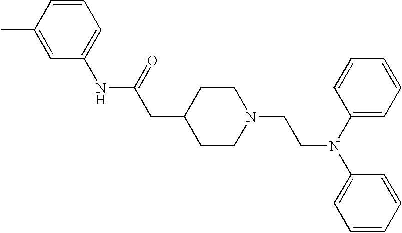 |
| 10 | 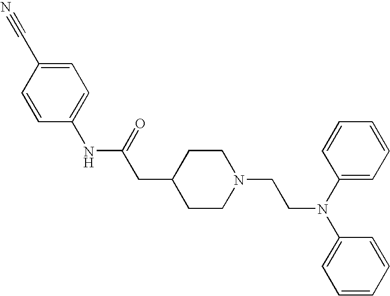 |
| 11 | 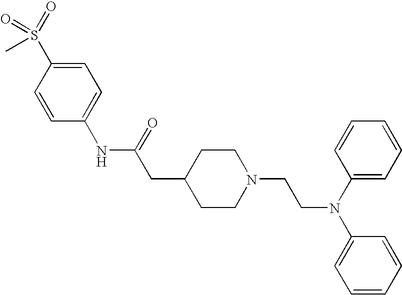 |
| 12 | 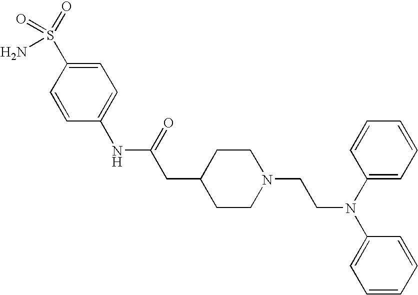 |
| 13 | 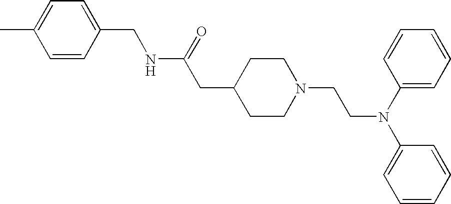 |
| 14 | 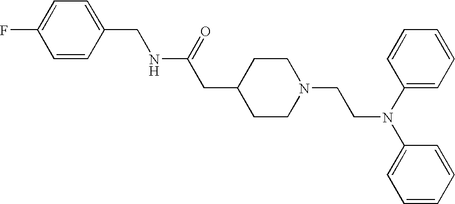 |
| 15 | 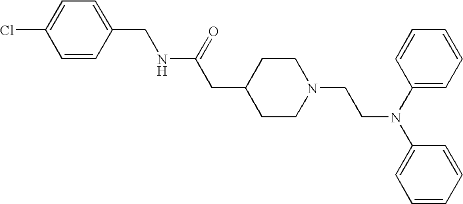 |
| 16 | 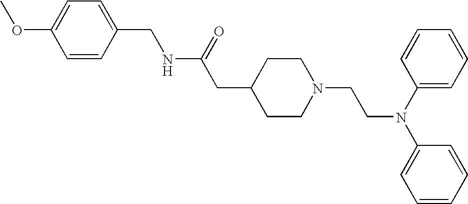 |
| 17 | 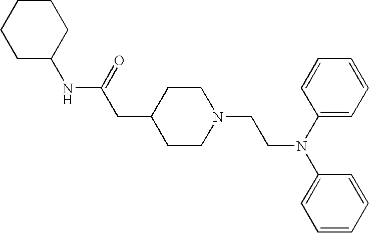 |
| 18 | 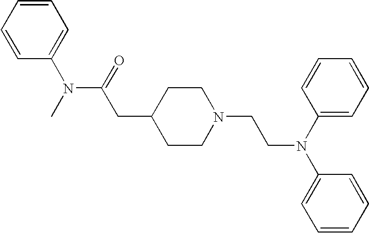 |
| 19 | 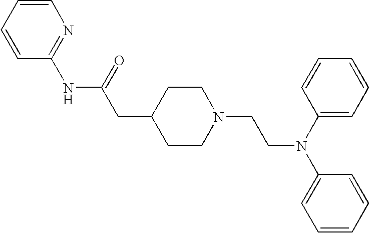 |
| 20 | 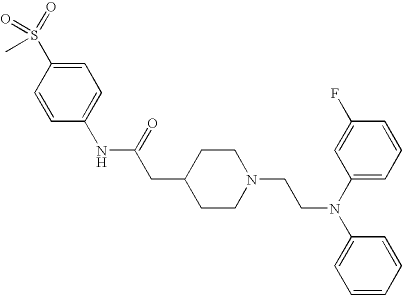 |
| 21 | 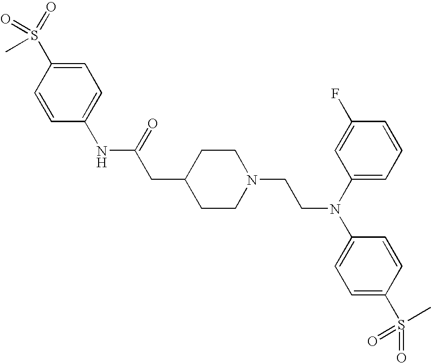 |
| 22 | 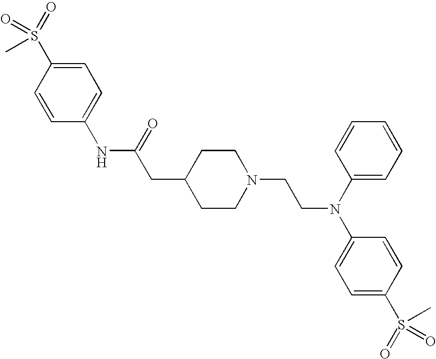 |
| 23 | 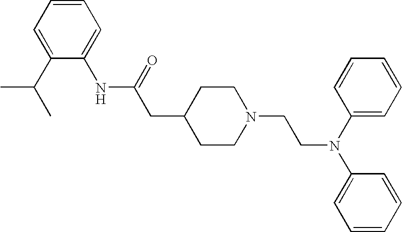 |
| 24 | 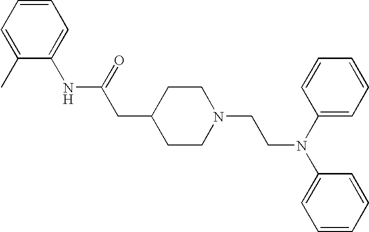 |
| 25 | 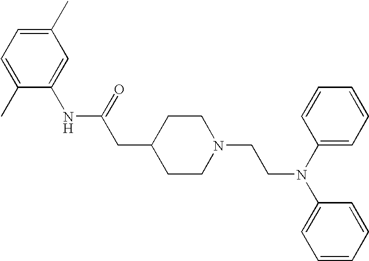 |
| 26 | 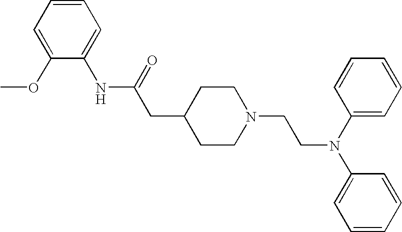 |
| 27 | 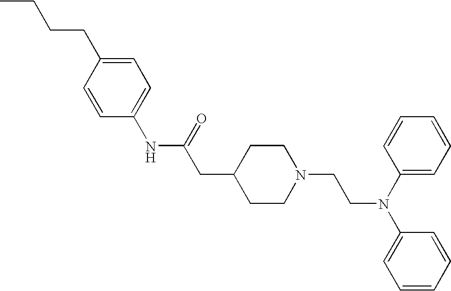 |
| 28 | 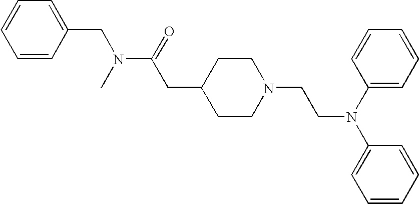 |
| 29 | 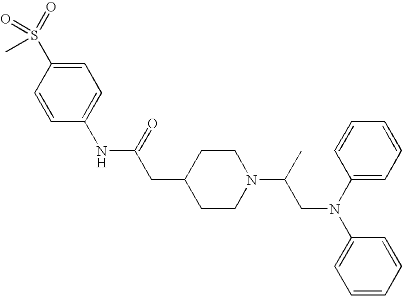 |
| 30 | 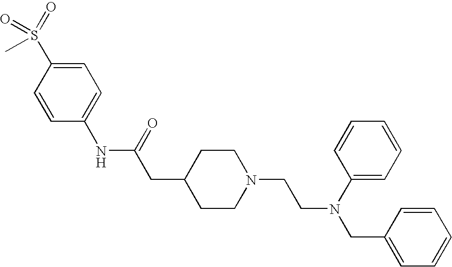 |
| 31 | 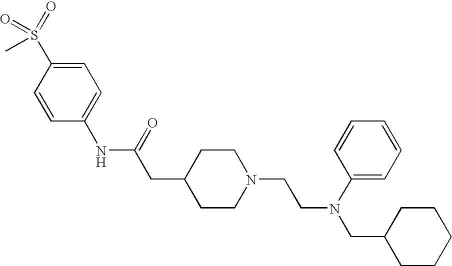 |
| 32 | 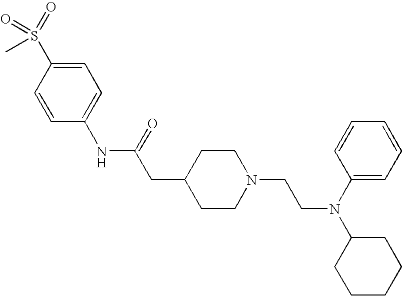 |
| 33 | 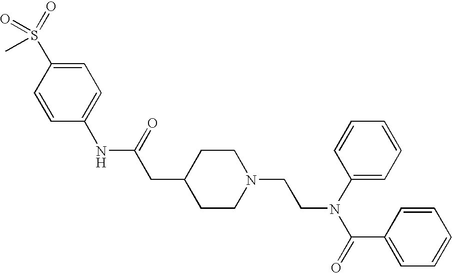 |
| 34 | 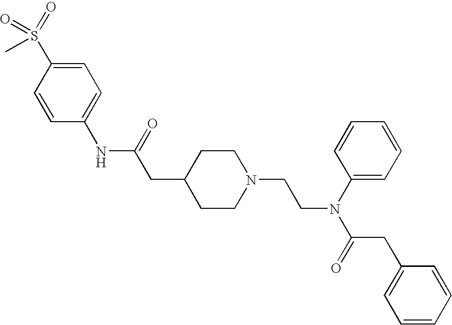 |
| 35 | 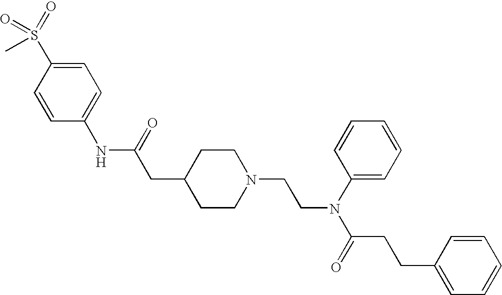 |
| 36 | 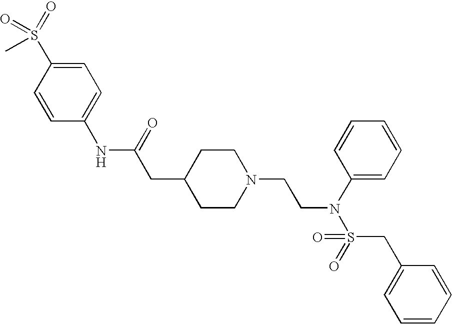 |
| 37 | 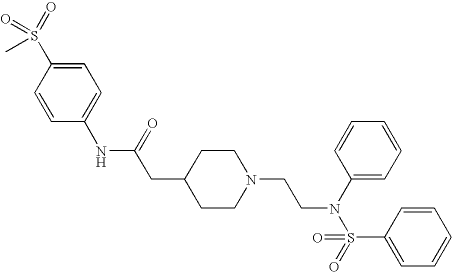 |
| 38 | 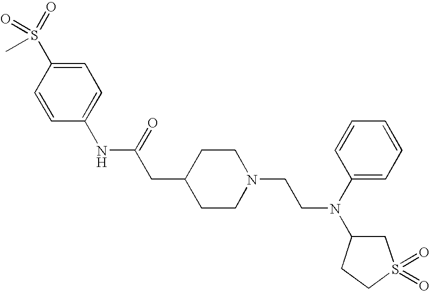 |
| 39 | 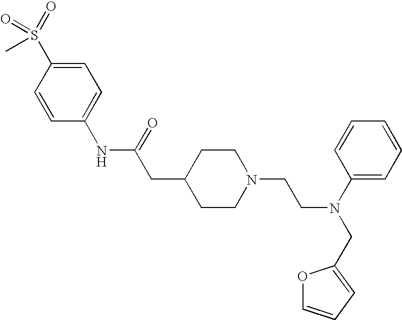 |
| 40 | 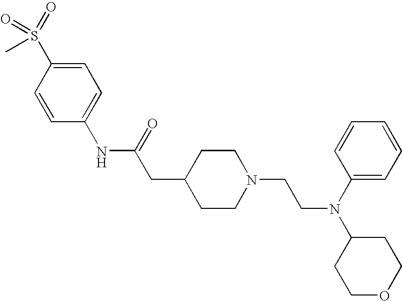 |
| 41 | 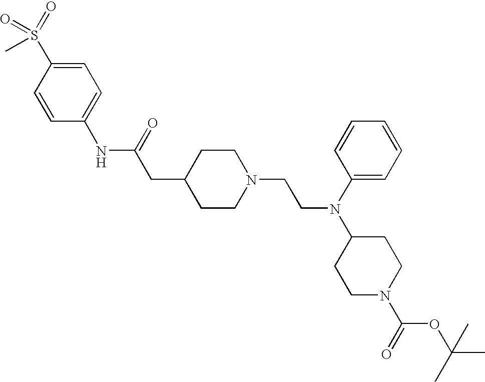 |
| 42 | 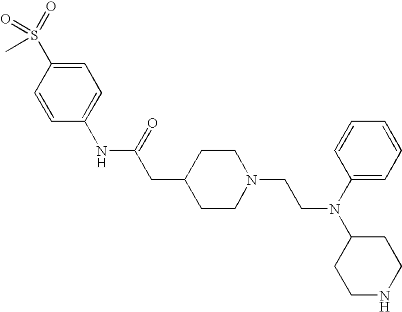 |
| 43 | 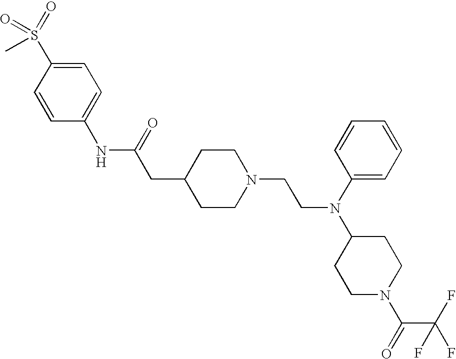 |
| 44 | 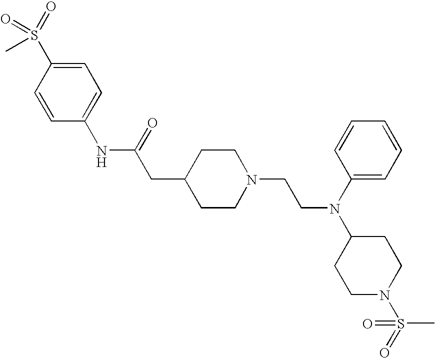 |
| 45 | 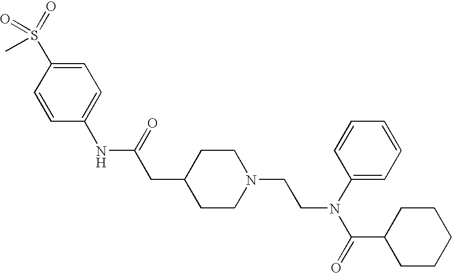 |
| 46 | 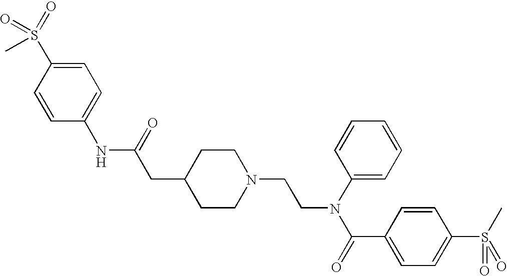 |
| 47 | 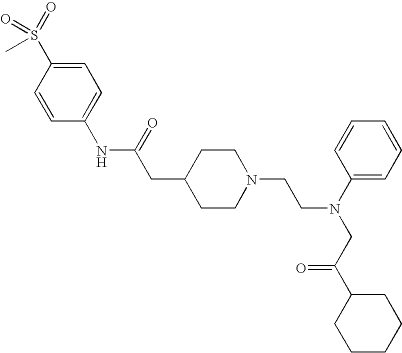 |
| 48 | 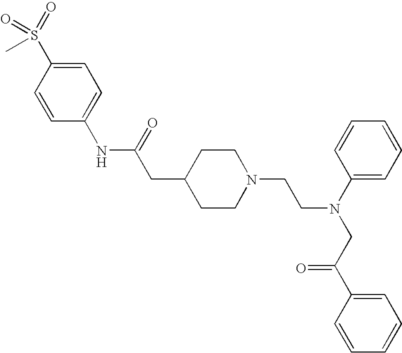 |
| 49 | 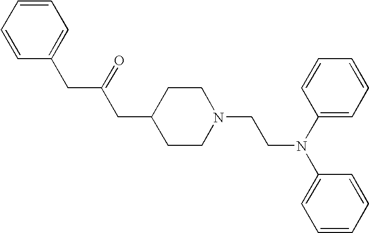 |
| 50 | 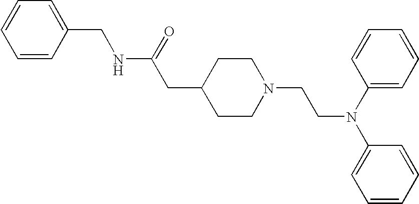 |
| 51 | 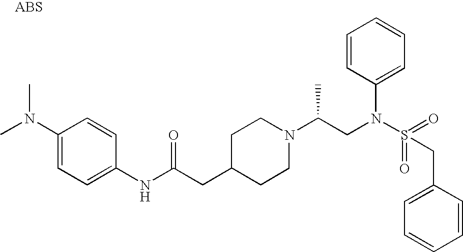 |
| 52 | 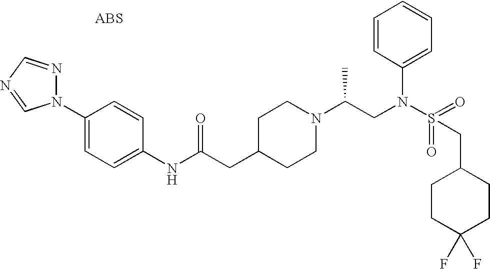 |
| 53 | 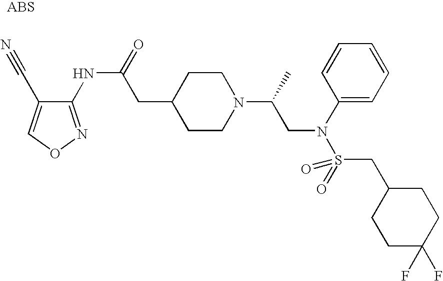 |
| 54 | 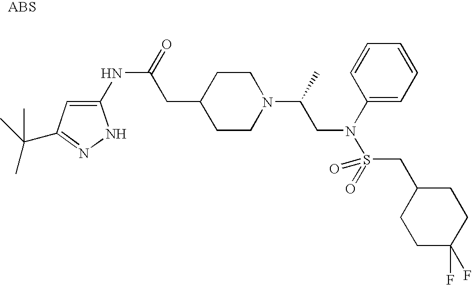 |
| 55 | 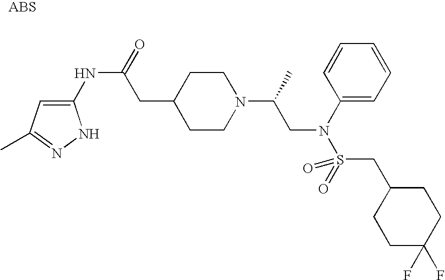 |
| 56 | 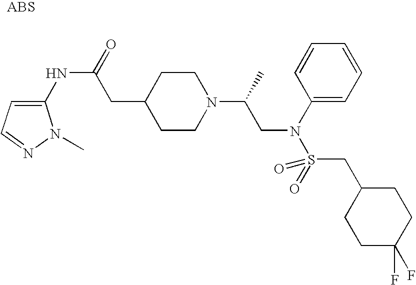 |
| 57 | 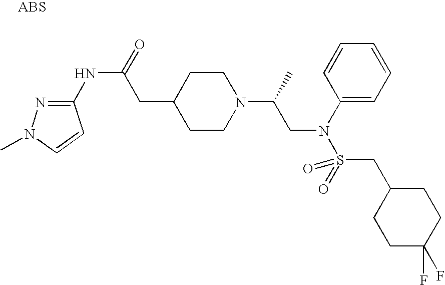 |
| 58 | 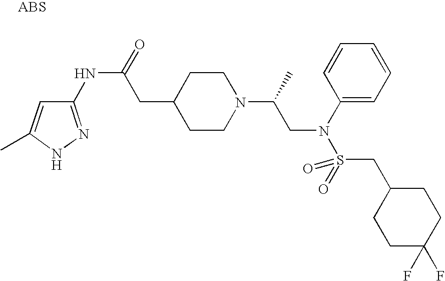 |
| 59 | 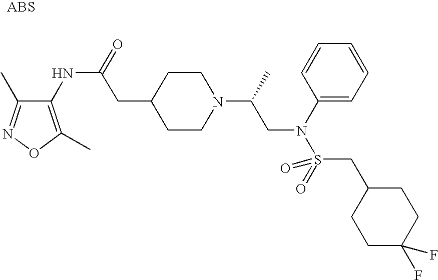 |
| 60 | 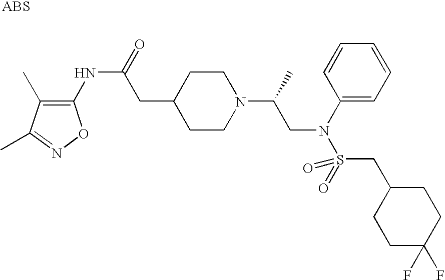 |
| 61 | 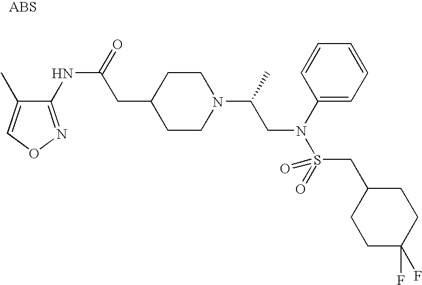 |
| 62 | 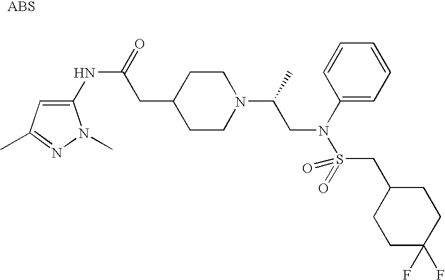 |
| 63 | 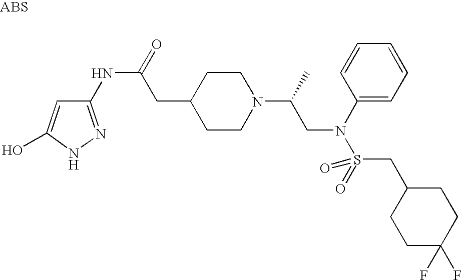 |
| 64 | 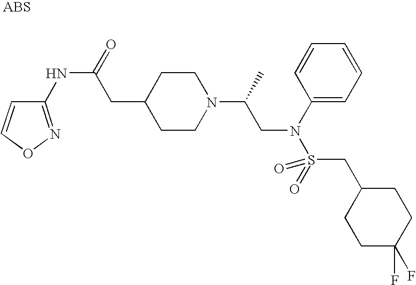 |
| 65 | 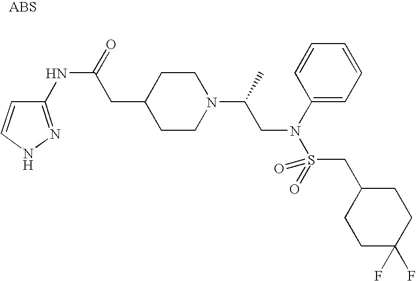 |
| 66 | 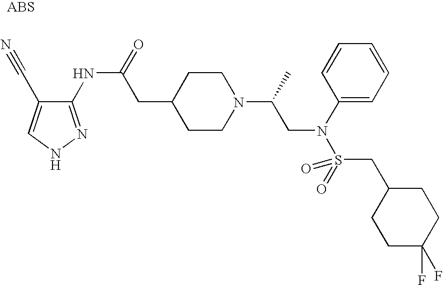 |
| 67 | 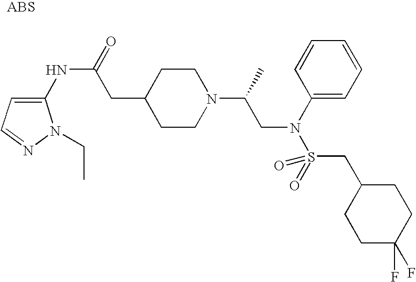 |
| 68 | 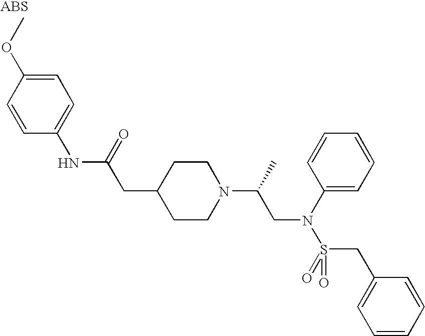 |
| 69 | 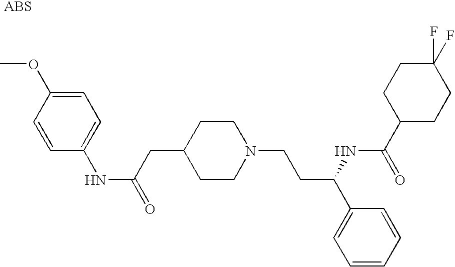 |
| 70 | 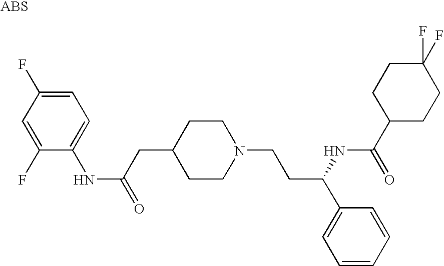 |
| 71 | 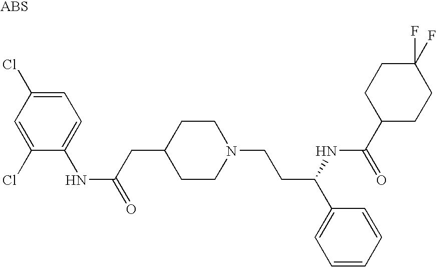 |
| 72 | 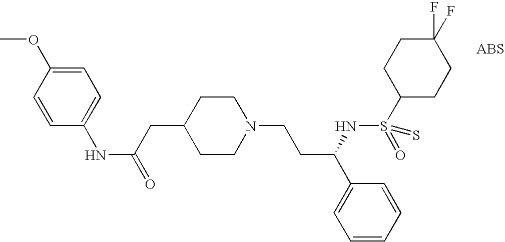 |
| 73 | 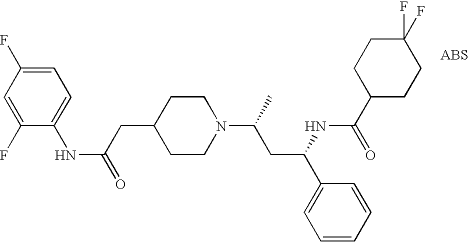 |
| 74 | 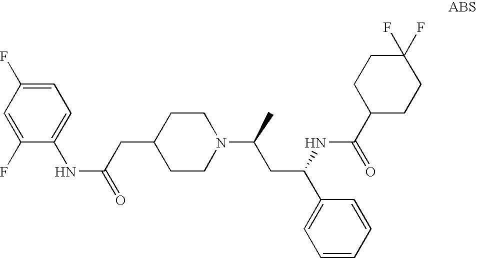 |
| 75 | 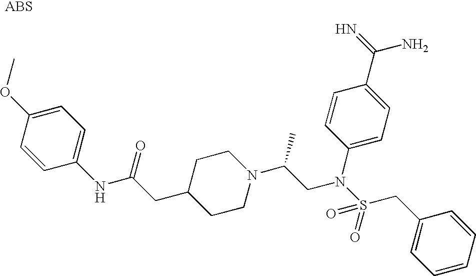 |
| 76 | 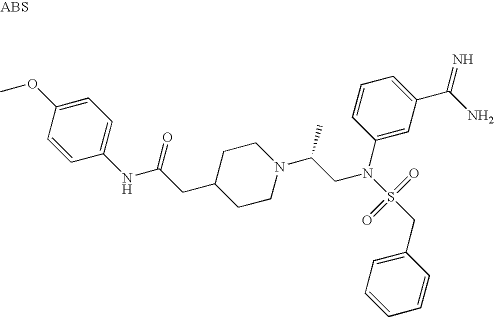 |
| 77 | 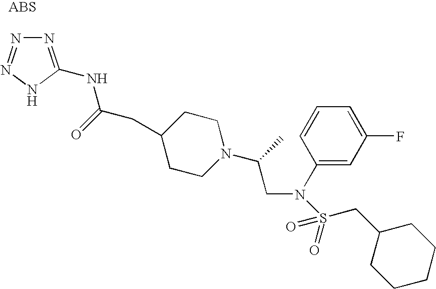 |
| 78 | 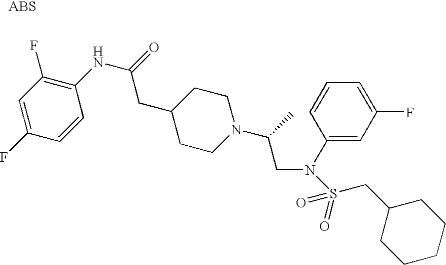 |
| 79 | 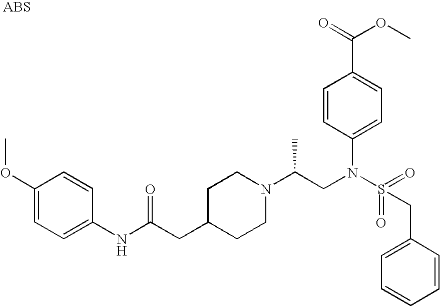 |
| 80 | 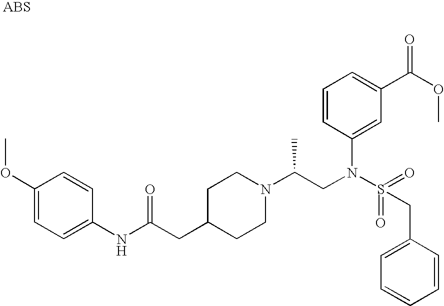 |
| 81 | 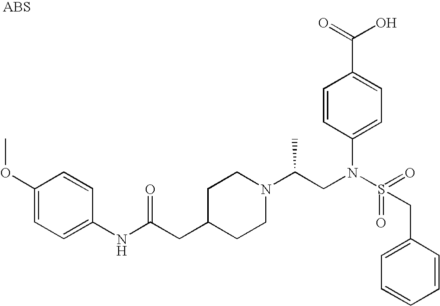 |
| 82 | 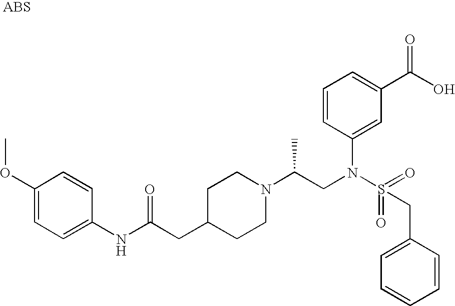 |
| 83 | 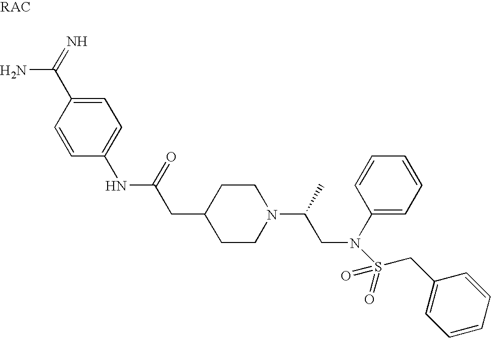 |
| 84 | 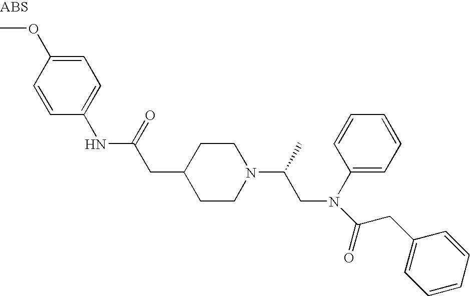 |
| 85 | 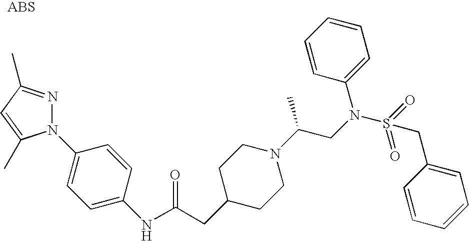 |
| 86 | 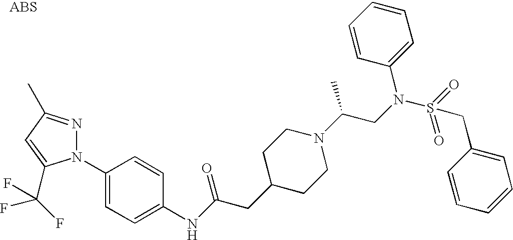 |
| 87 | 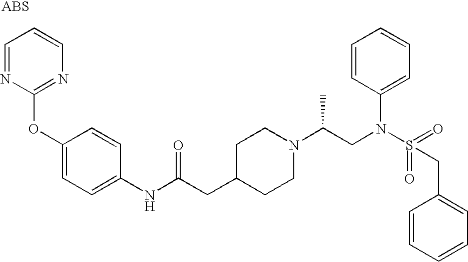 |
| 88 | 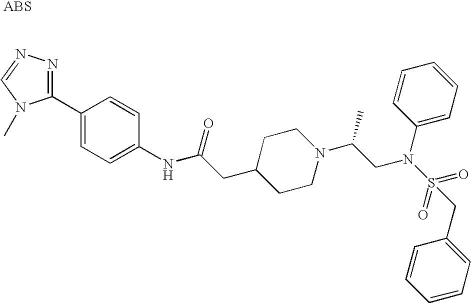 |
| 89 | 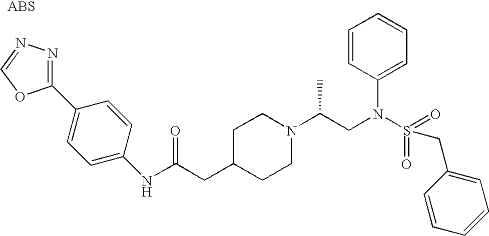 |
| 90 | 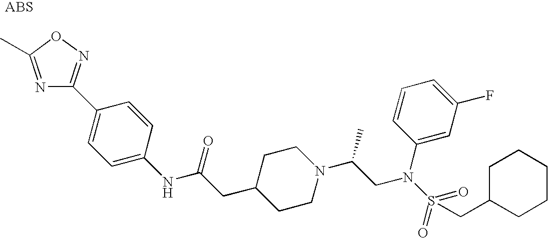 |
| 91 | 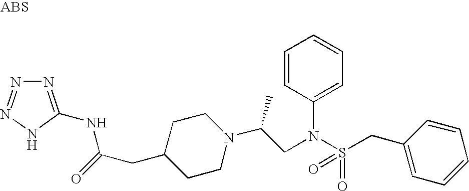 |
| 92 | 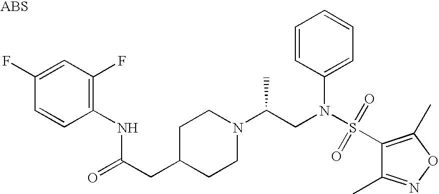 |
| 93 | 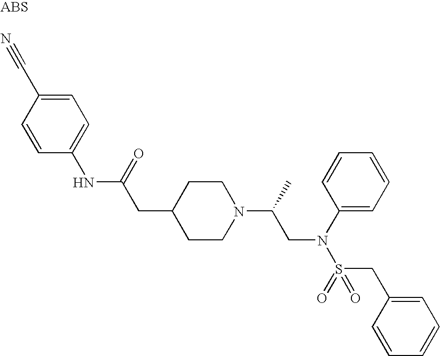 |
| 94 | 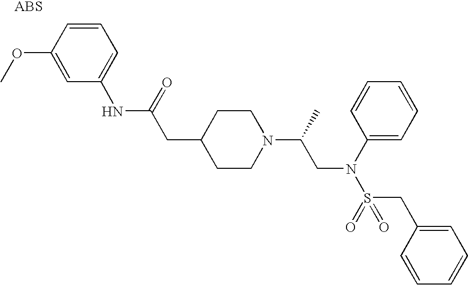 |
| 95 | 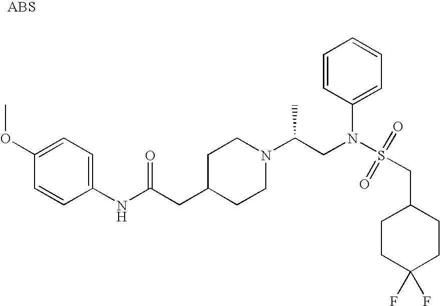 |
| 96 | 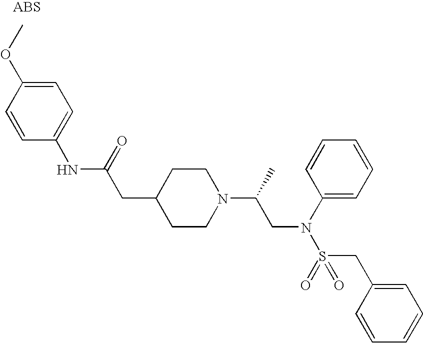 |
| 97 | 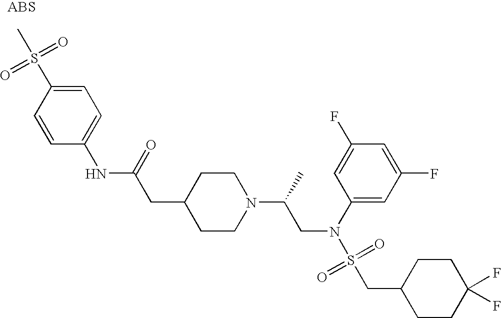 |
| 98 | 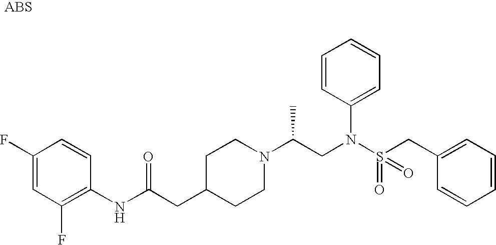 |
| 99 | 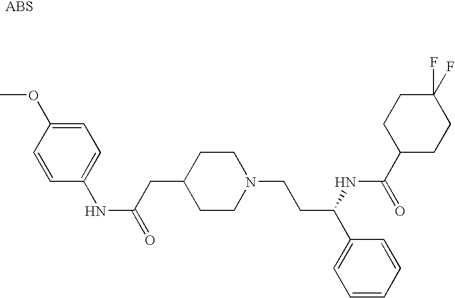 |
| 100 | 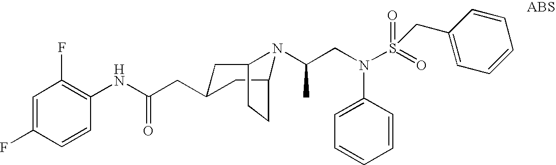 |
| 101 | 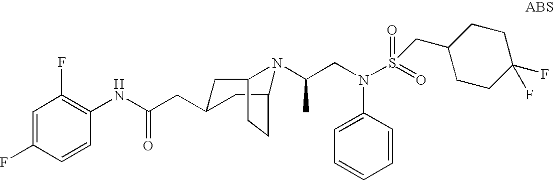 |
| 102 | 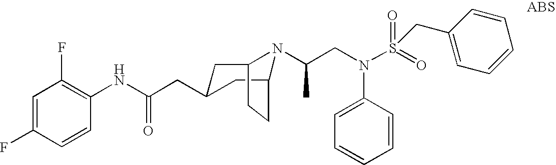 |

The compounds of formula I can be prepared by different ways with reactions known by the person skilled in the art. Reaction schemes I to III (Example section), illustrate by way of example different possible approaches.

The invention further provides the use of the compounds of the invention or pharmaceutically acceptable salts or solvates thereof as modulators of chemokine receptor activity, especially as modulators of CCR5 activity. In a preferred embodiment the compounds of Formula I or pharmaceutically acceptable salts or solvates thereof are used as CCR5 antagonists or CCR5 agonists.

In still another embodiment the administration of agonists only, may be advantageous in comparison with the antagonist approach because a CCR5-agonist may reduce the generation of certain types of HIV variants. Indeed, agonist molecules will promote CCR5 receptor disappearance from the cell surface by inducing its internalization. This would prevent the emergence of variants of the type able to bind the antagonist-bound CCR5, as previously observed for example with the small molecule antagonist Maraviroc (Westby M et al (2007) J Virol 81(5):2359-71). Avoiding generation of HIV variants, for example variants of the type able to bind the antagonist-bound CCR5, and therefore avoiding therapeutic resistance is one of the goal of this invention.

Accordingly, in a particularly preferred embodiment, the invention relates to the use of compounds of formula I, Ia, Ib, Ic, Id and Id′, or pharmaceutically acceptable salts or solvates thereof, as CCR5 agonists. Examples of such compounds are represented in table 2:

TABLE 2

| Compound | Structure |
|---|---|
| 1 | 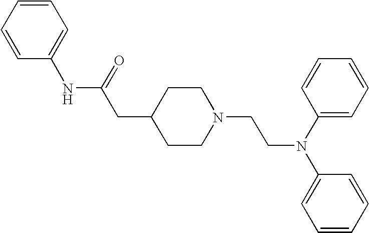 |
| 2 | 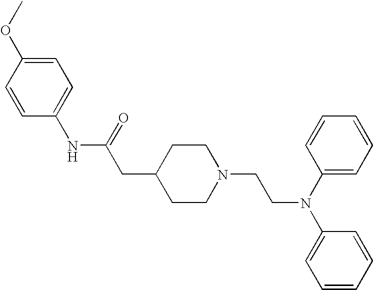 |
| 3 | 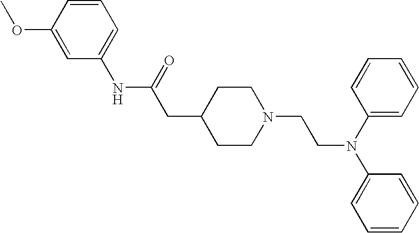 |
| 4 | 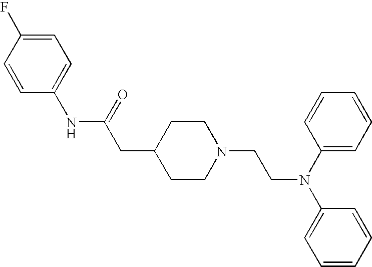 |
| 5 | 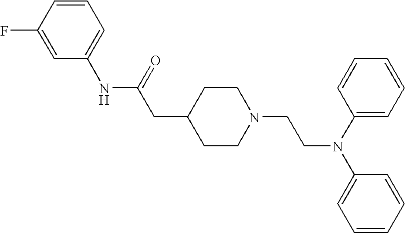 |
| 6 | 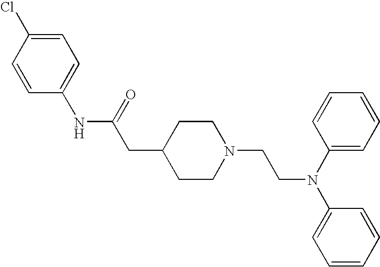 |
| 7 | 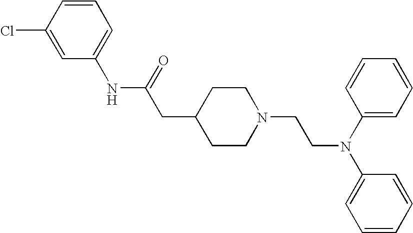 |
| 8 | 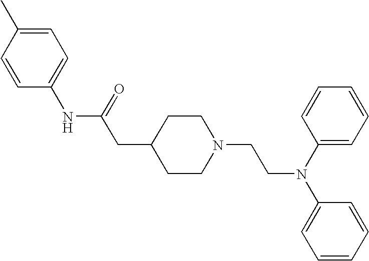 |
| 9 | 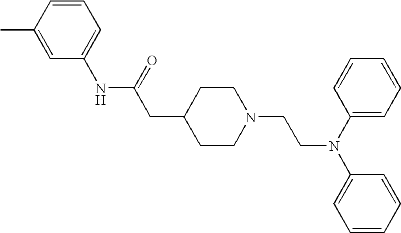 |
| 12 | 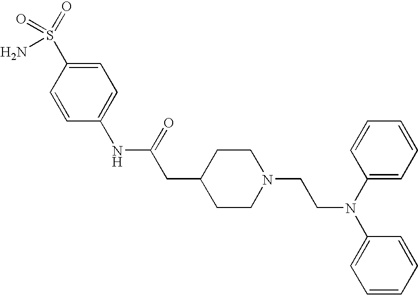 |
| 15 | 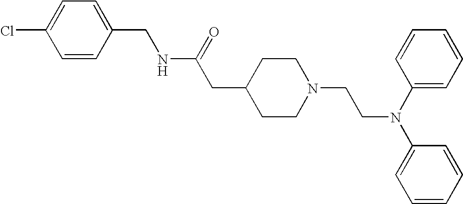 |
| 16 | 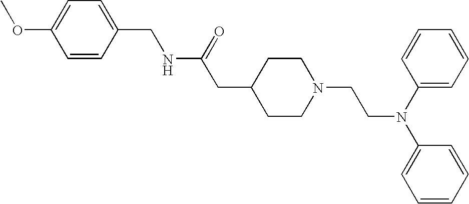 |
| 17 | 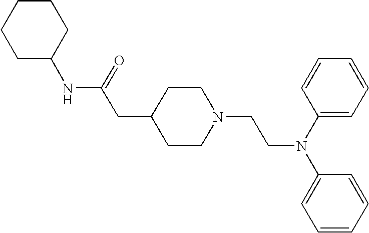 |
| 18 | 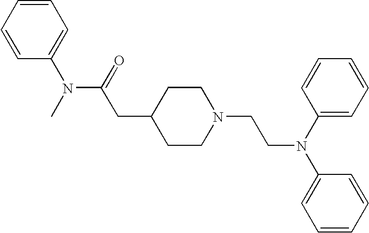 |
| 19 | 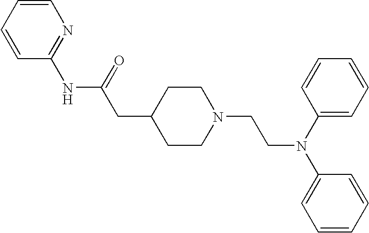 |
| 23 | 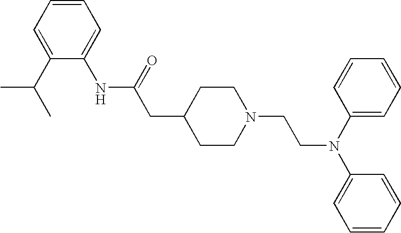 |
| 27 | 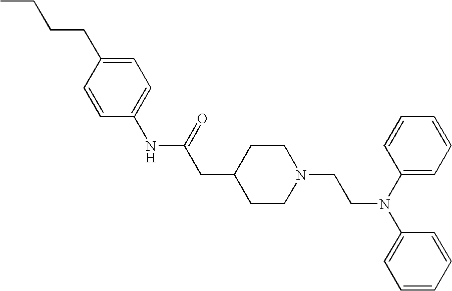 |
| 28 | 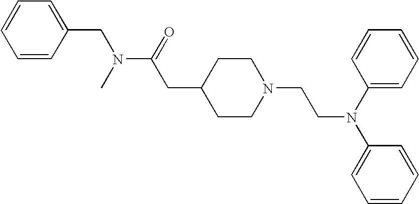 |
| 49 | 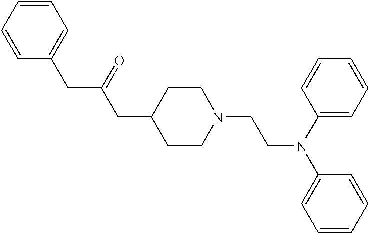 |
| 50 | 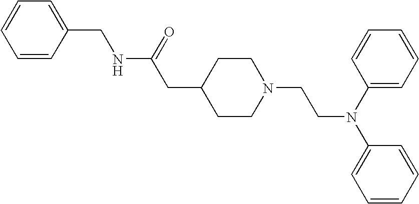 |
| 51 | 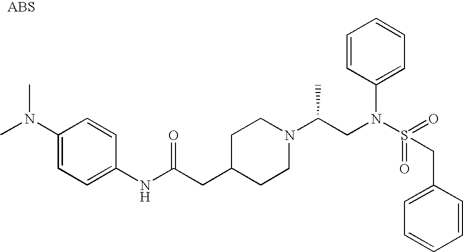 |
| 52 | 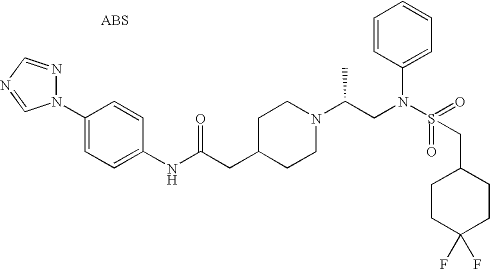 |
| 53 | 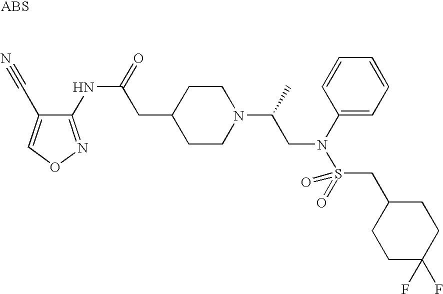 |
| 54 | 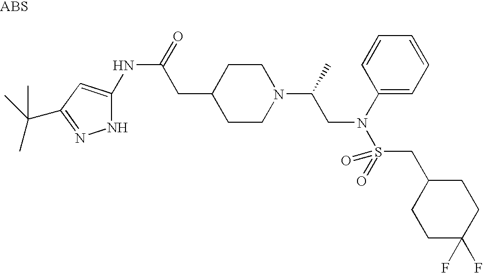 |
| 55 | 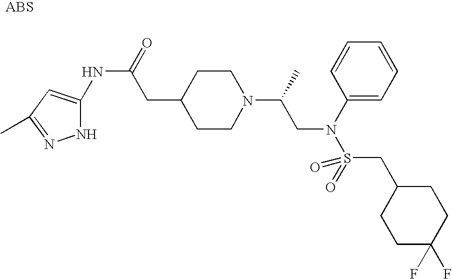 |
| 56 | 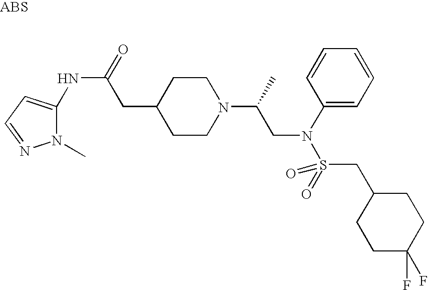 |
| 57 | 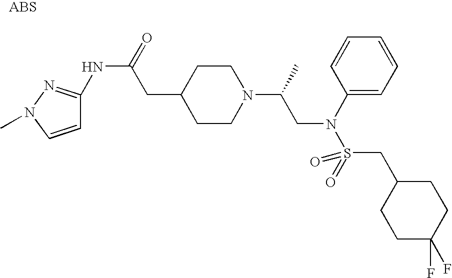 |
| 58 | 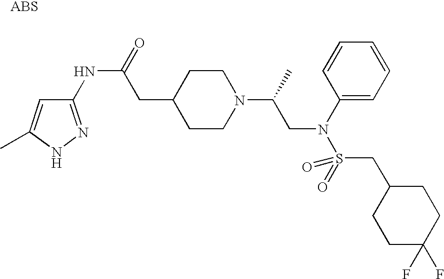 |
| 59 | 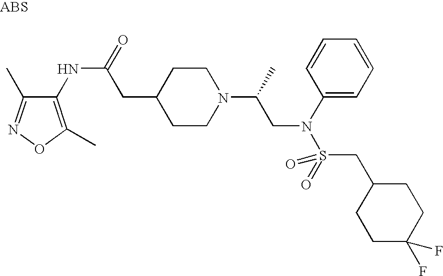 |
| 60 | 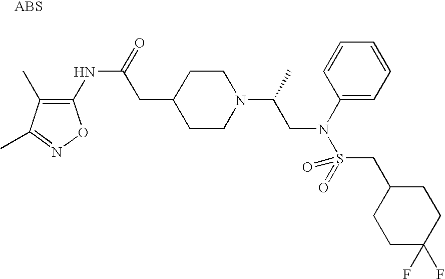 |
| 61 | 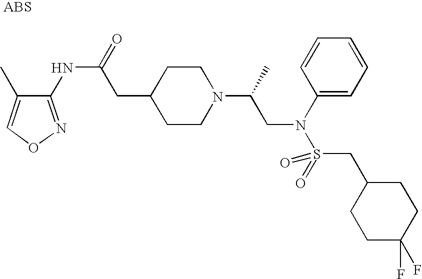 |
| 62 | 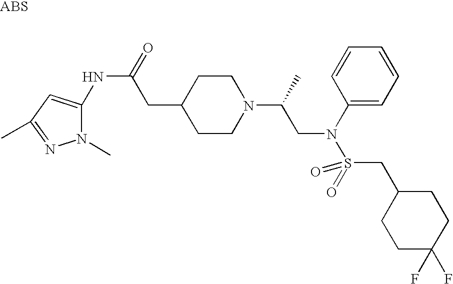 |
| 63 | 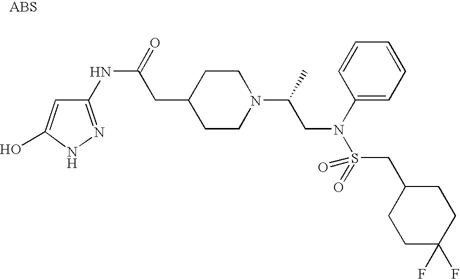 |
| 64 | 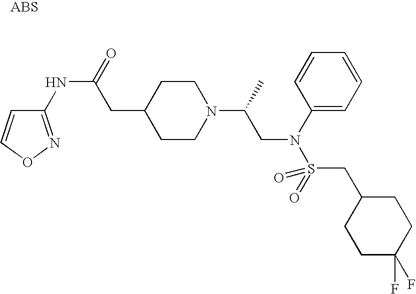 |
| 65 | 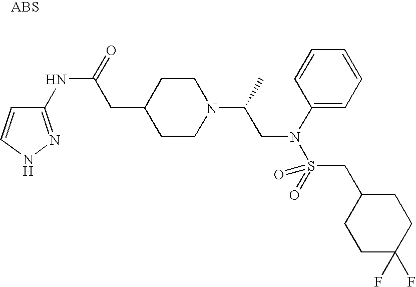 |
| 66 | 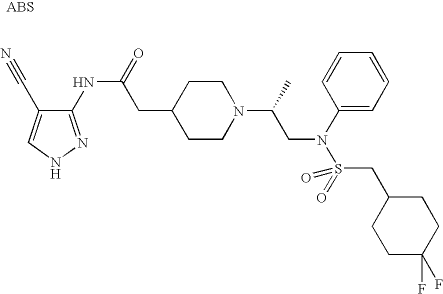 |
| 67 | 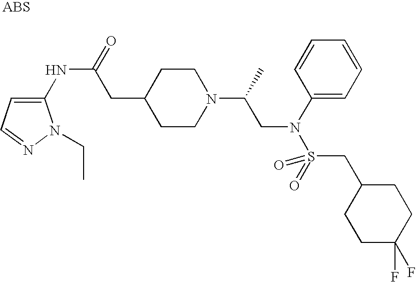 |
| 68 | 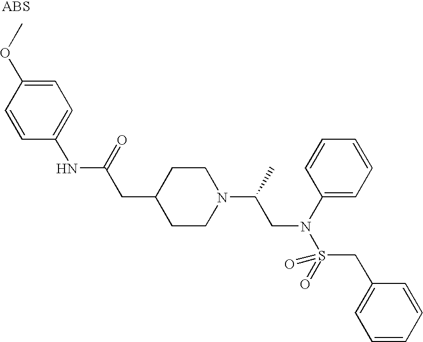 |
| 69 | 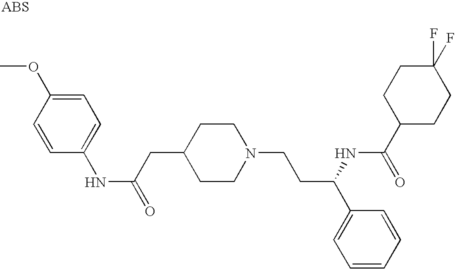 |
| 70 | 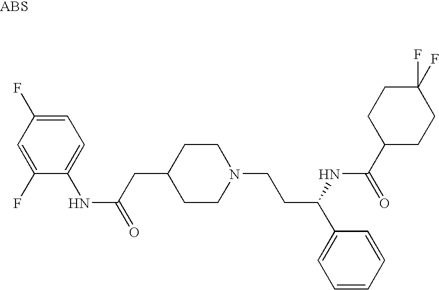 |
| 71 | 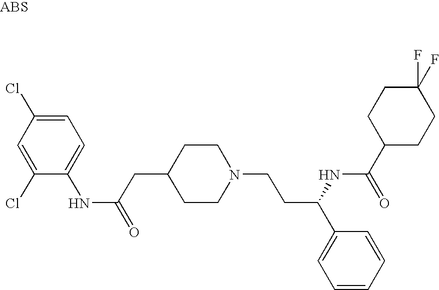 |
| 72 | 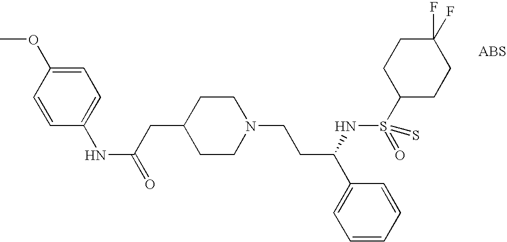 |
| 73 | 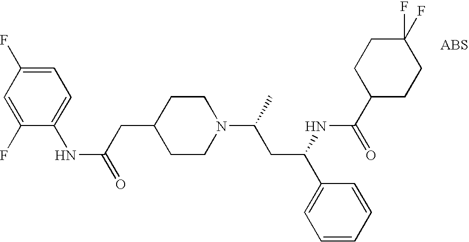 |
| 74 | 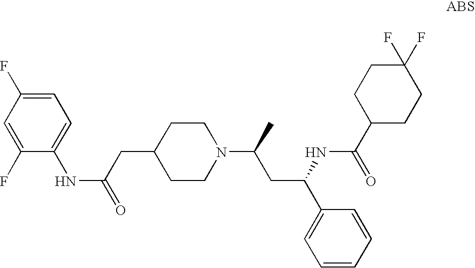 |
| 75 | 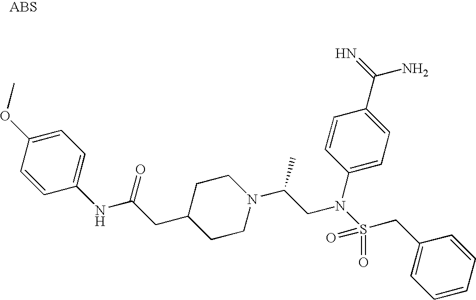 |
| 76 | 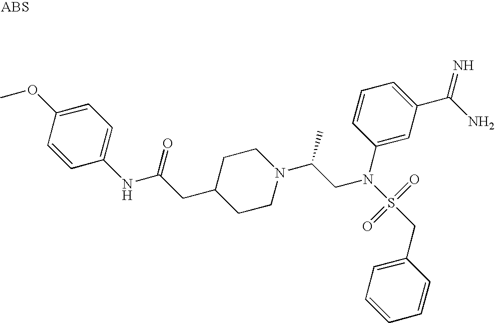 |
| 77 | 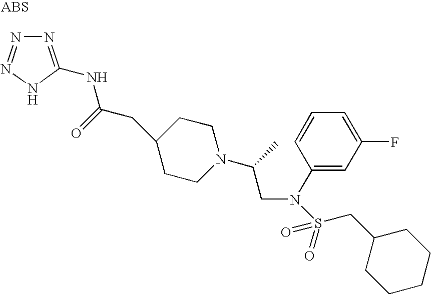 |
| 78 | 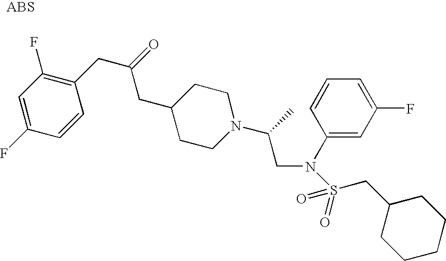 |
| 79 | 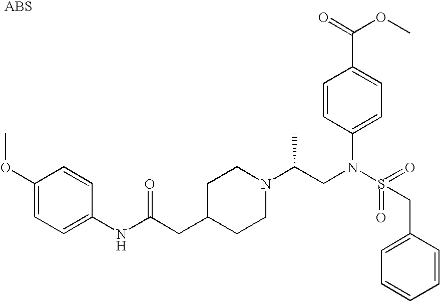 |
| 80 | 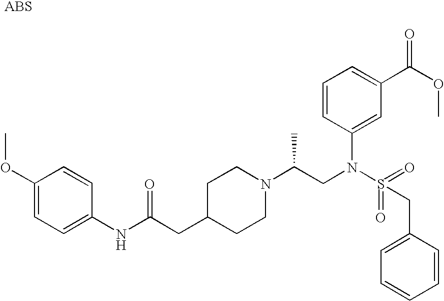 |
| 81 | 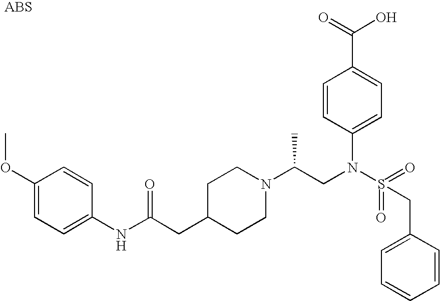 |
| 82 | 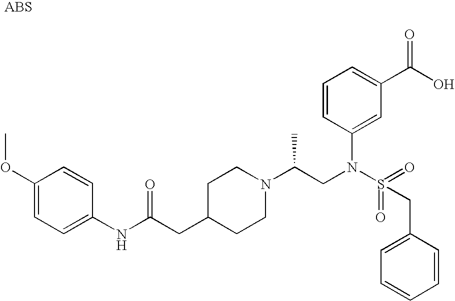 |
| 83 | 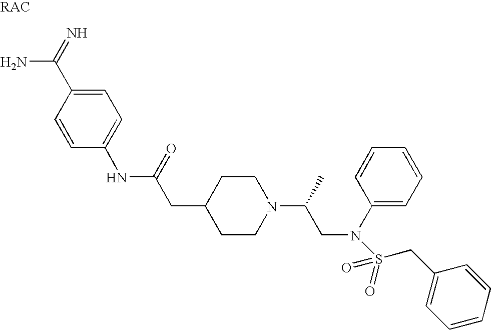 |
| 84 | 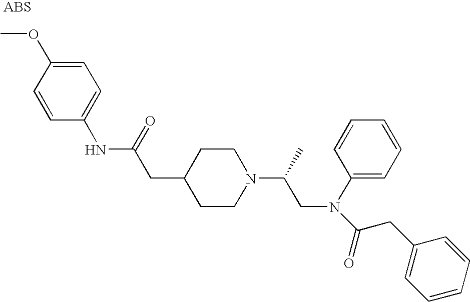 |
| 85 | 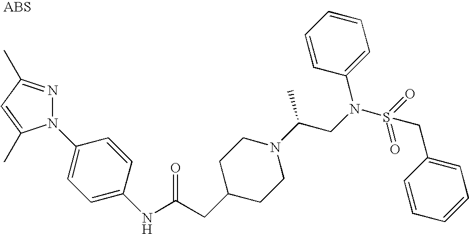 |
| 86 | 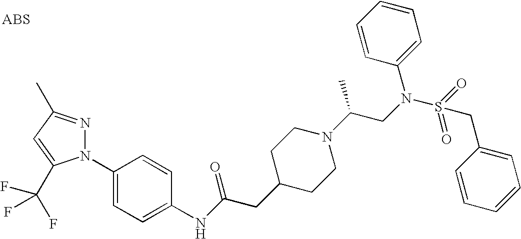 |
| 87 | 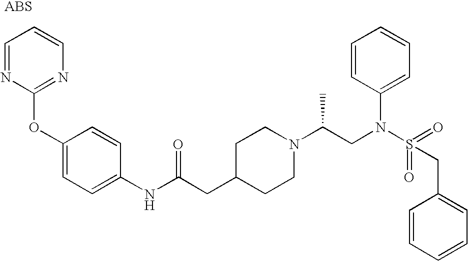 |
| 88 | 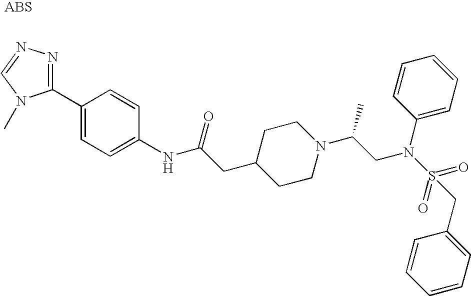 |
| 89 | 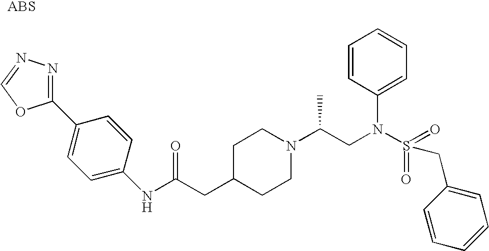 |
| 90 | 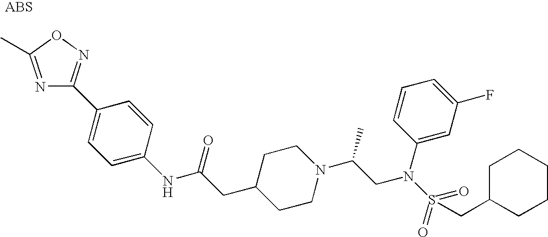 |
| 91 | 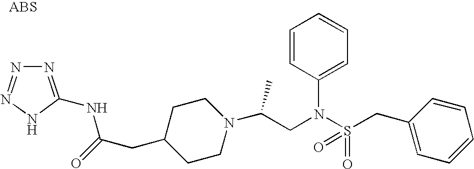 |
| 92 | 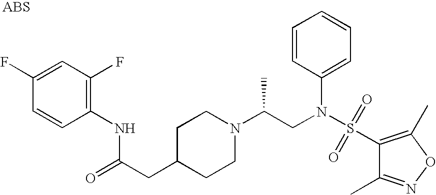 |
| 93 | 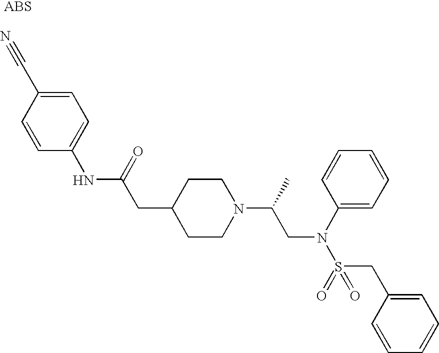 |
| 94 | 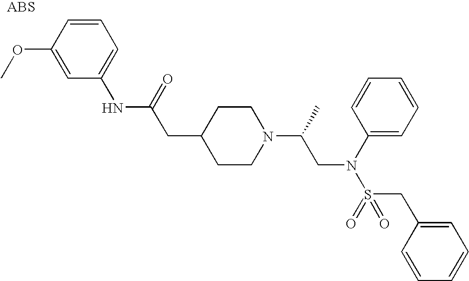 |
| 95 | 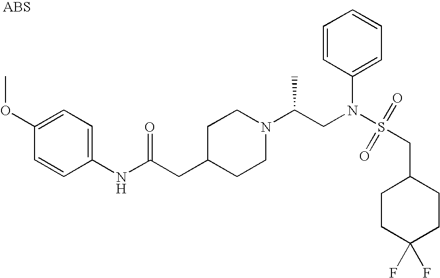 |
| 96 | 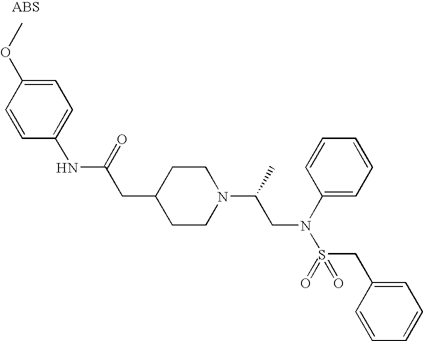 |
| 97 | 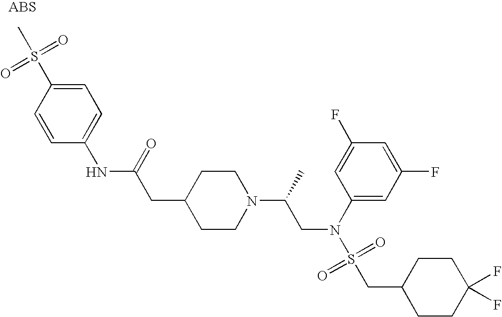 |
| 98 | 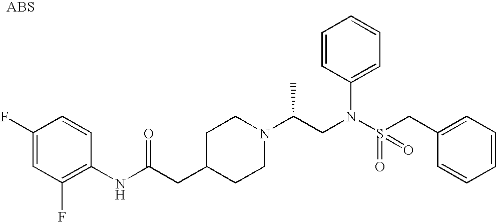 |
| 99 | 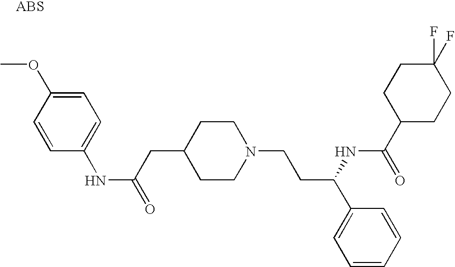 |
| 100 | 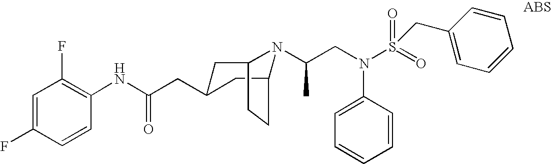 |
| 101 | 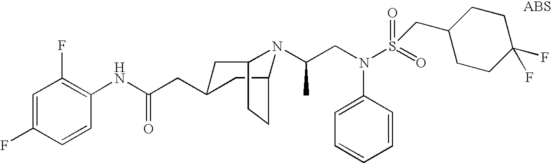 |
| 102 | 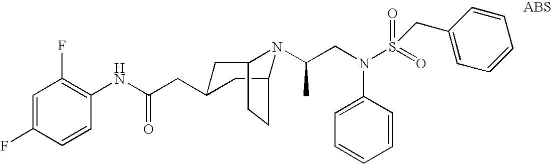 |
| 52 | 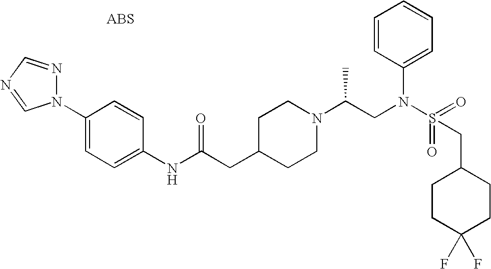 |
| 53 | 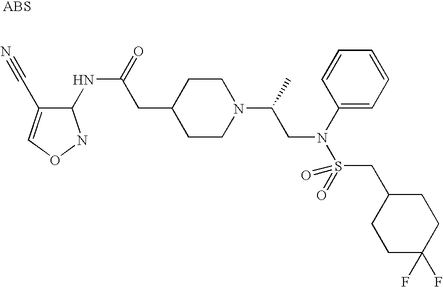 |
| 54 | 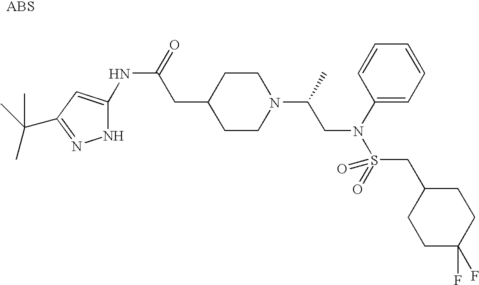 |
| 55 | 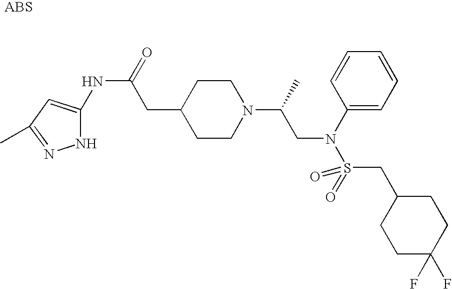 |
| 56 | 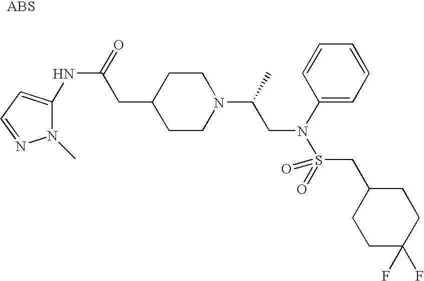 |
| 57 | 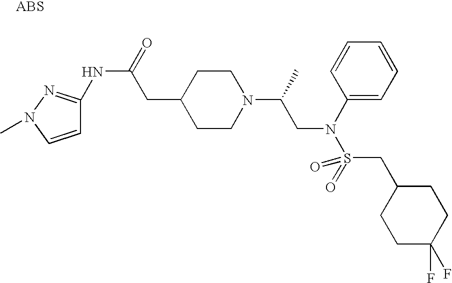 |
| 58 | 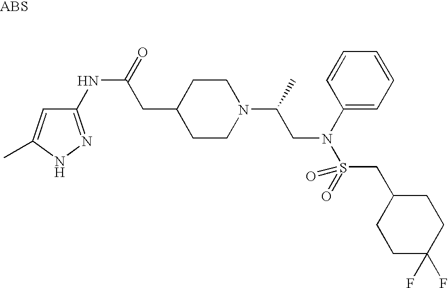 |
| 59 | 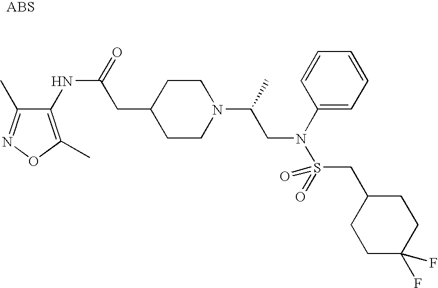 |
| 60 | 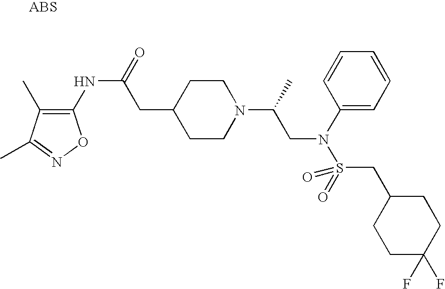 |
| 61 | 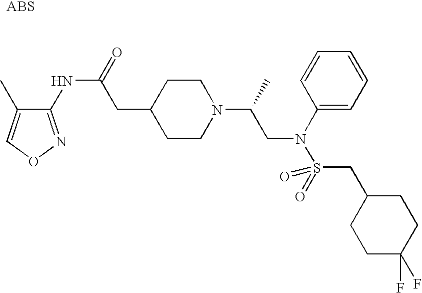 |
| 62 | 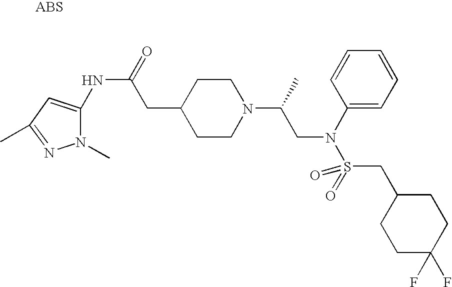 |
| 63 | 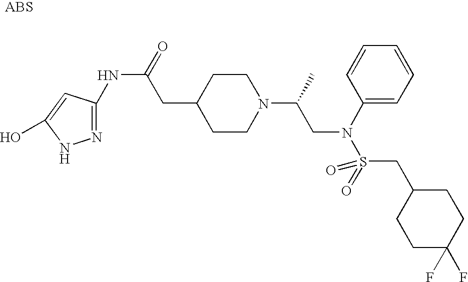 |
| 64 | 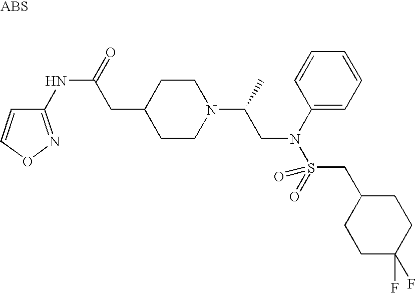 |
| 65 | 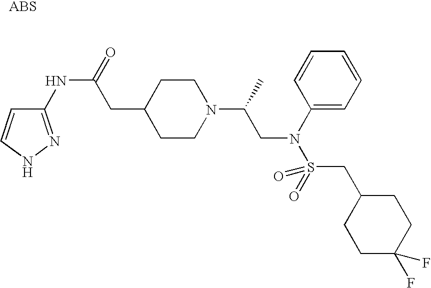 |
| 66 | 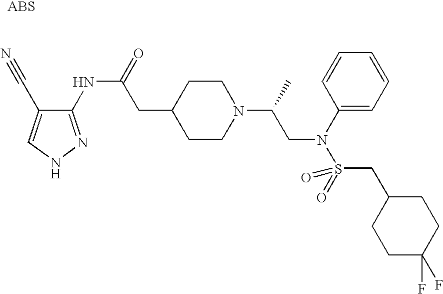 |
| 67 | 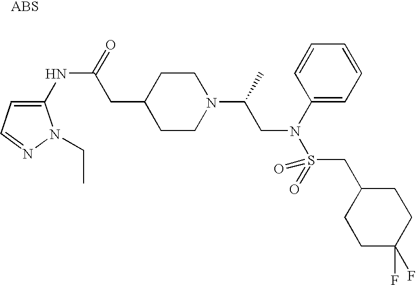 |
| 68 | 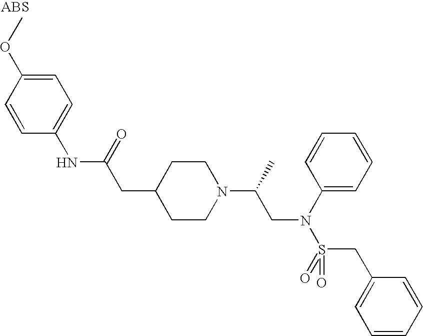 |
| 69 | 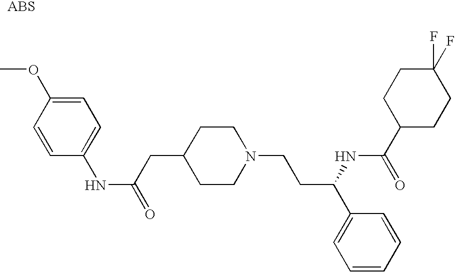 |
| 70 | 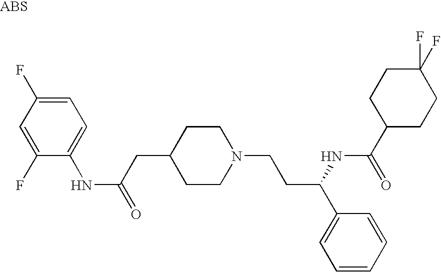 |
| 71 | 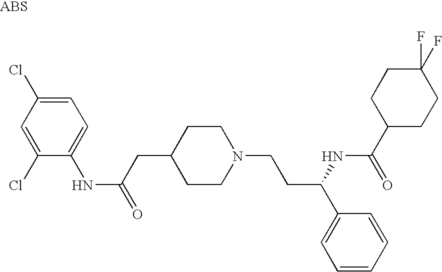 |
| 72 | 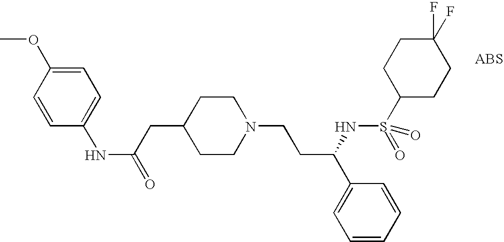 |
| 73 | 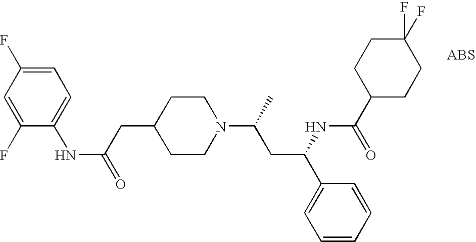 |
| 74 | 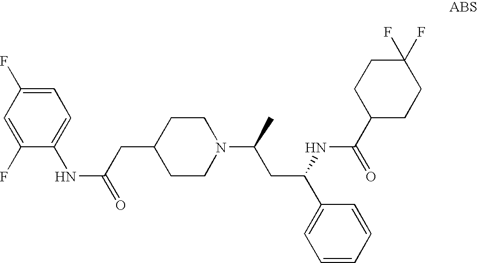 |
| 75 | 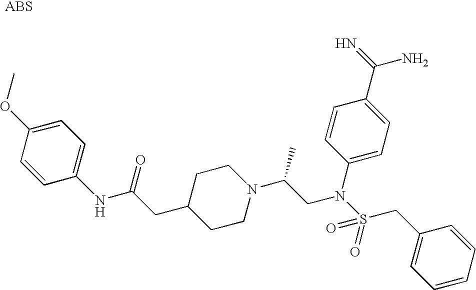 |
| 76 | 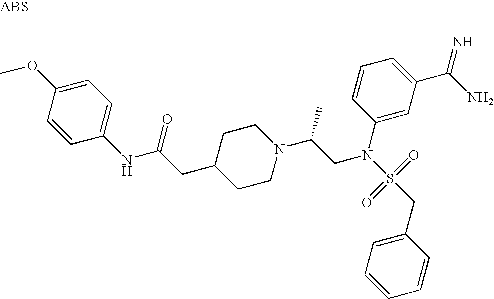 |
| 77 | 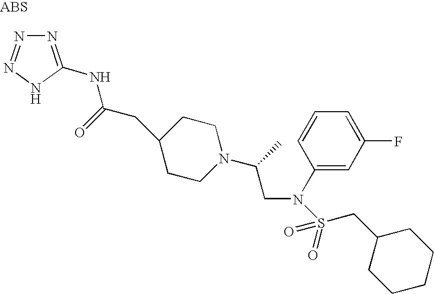 |
| 78 | 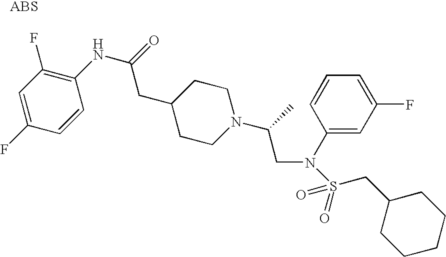 |
| 79 | 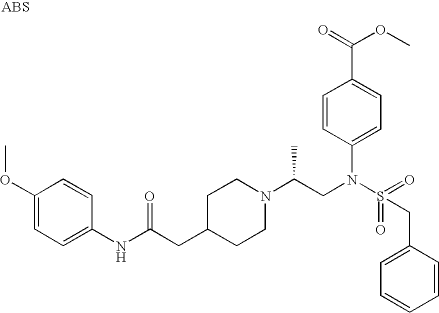 |
| 80 | 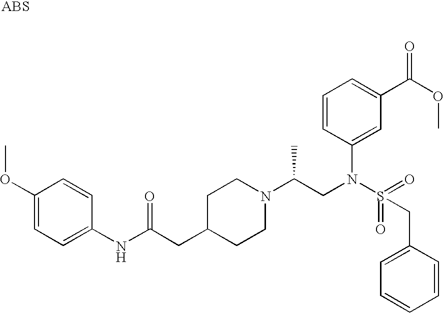 |
| 81 | 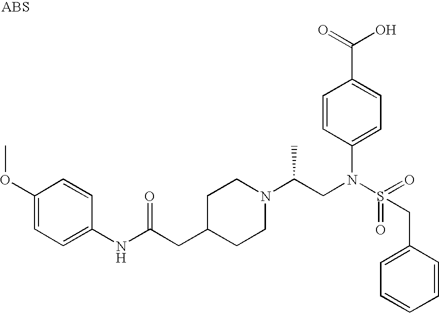 |
| 82 | 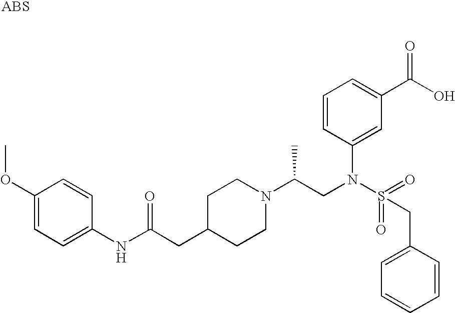 |
| 83 | 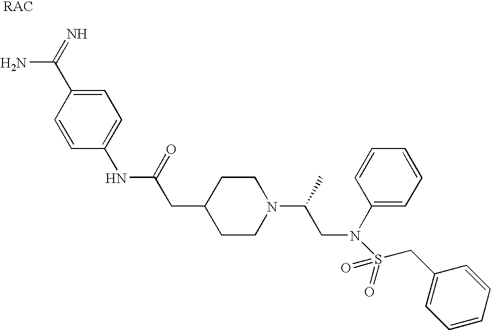 |
| 84 | 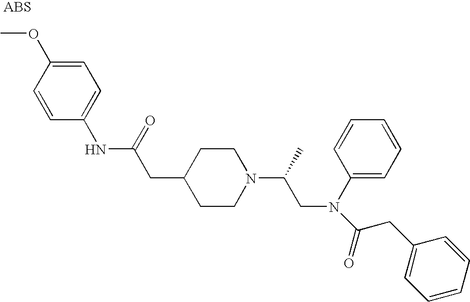 |
| 85 | 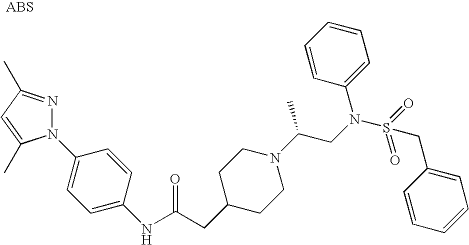 |
| 86 | 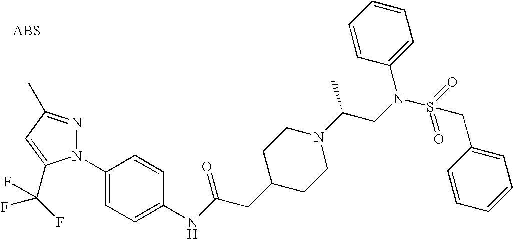 |
| 87 | 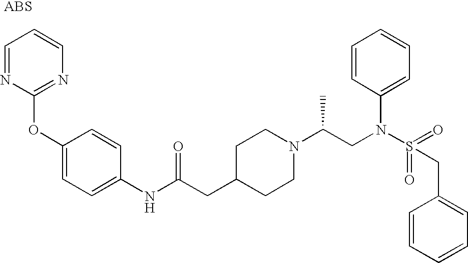 |
| 88 | 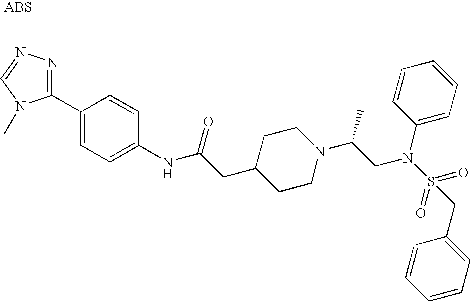 |
| 89 | 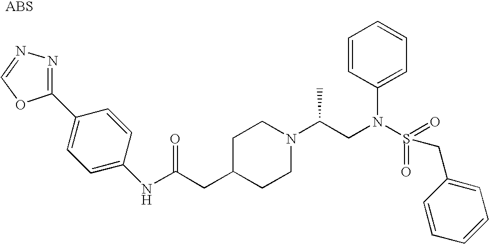 |
| 90 | 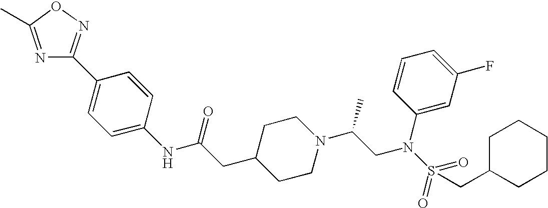 |
| 91 | 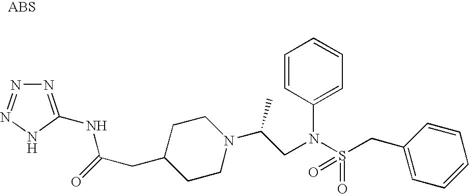 |
| 92 | 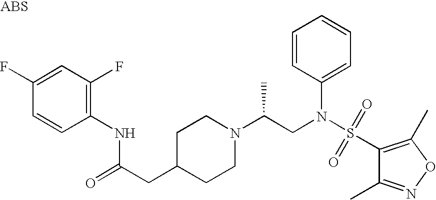 |
| 93 | 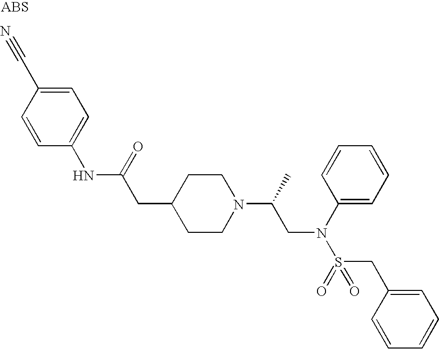 |
| 94 | 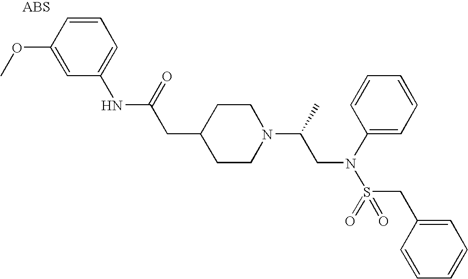 |
| 95 | 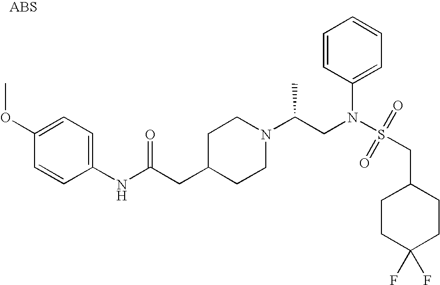 |
| 96 | 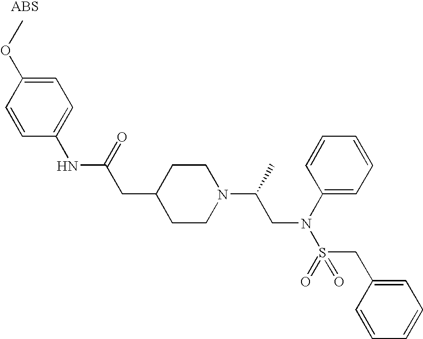 |
| 97 | 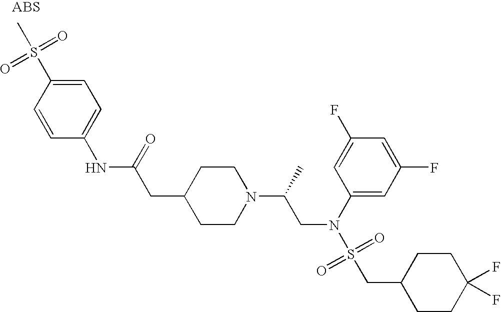 |
| 98 | 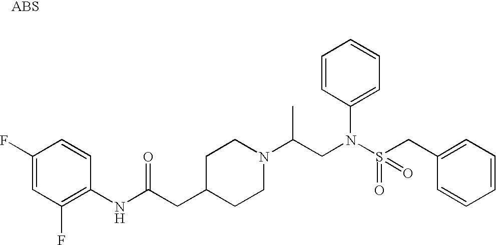 |
| 99 | 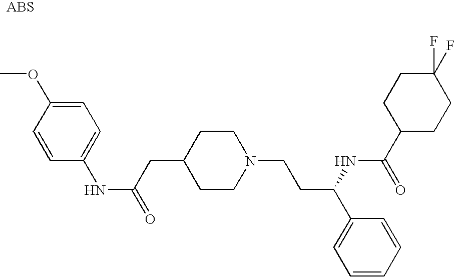 |
| 100 | 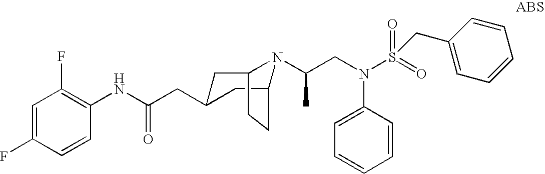 |
| 101 | 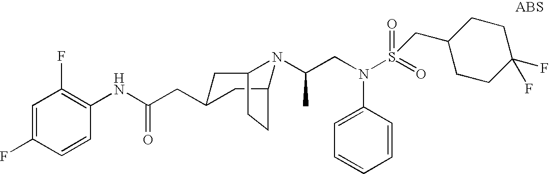 |
| 102 | 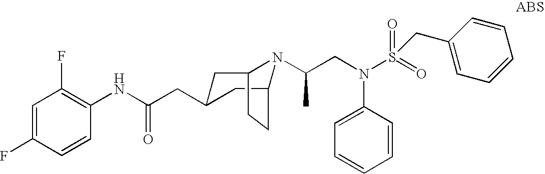 |

### [Applications]

The invention further provides methods for the treatment or prevention of autoiimune, inflammatory, infectious, proliferative or hyperproliferative diseases, or immunologically-mediated diseases (including rejection of transplanted organs, or tissues and Acquired Immunodeficiency Syndrome (AIDS)); examples of these conditions are:
(1) (the respiratory tract) obstructive diseases of airways including: chronic obstructive pulmonary disease (COPD) (such as irreversible COPD); pulmonary fibrosis; asthma {such as bronchial, allergic, intrinsic, extrinsic or dust asthma, particularly chronic or inveterate asthma (for example late asthma or airways hyper-responsiveness)); bronchitis (such as eosinophilic bronchitis}; acute, allergic, atrophic rhinitis or chronic rhinitis including rhinitis caseosa, hypertonic rhinitis, rhinitis purulenta, rhinitis sicca or rhinitis medicamentosa; membranous rhinitis including croupous, fibrinous or pseudomembranous rhinitis or scrofoulous rhinitis; seasonal rhinitis including rhinitis nervosa (hay fever) or vasomotor rhinitis; sarcoidosis; farmer's lung and related diseases; nasal polyposis; fibroid lung or idiopathic interstitial pneumonia;
(2) (bone and joints) arthrides including rheumatic, infectious, autoimmune, seronegative spondyloarthropathies (such as ankylosing spondylitis, psoriatic arthritis or Reiter's disease), Behçet's disease, Sjogren's syndrome or systematic sclerosis;
(3) (skin and eyes) psoriasis, atopic dermatitis, contact dermatitis or other eczematous dermitides, seborrhoetic dermatitis, Lichen planus, Phemphigus, bullous Phemphigus, Epidermolysis bullosa, urticaria, angiodermas, vasiculitides erythermas, cutaneaous eosinophilias, uveitis, Alopecia greata or vernal conjunctivitis;
(4) (gastrointestinal tract) Coeliac disease, proctitis, eosinophilic gastro-enteritis, mastocytosis, Crohn's disease, ulcerative colitis, irritable bowel disease or food-related allergies which have effects remote from the gut (for example migraine, rhinitis or eczema);
(5) (Allorgraft rejection) acute and chronic following, for example, transplantation of kidney, heart, liver, lung bone marrow, skin or cornea; or chronic graft versus host disease; and/or
(6) (other tissues or diseases) Alzheimer's disease, multiple sclerosis, atherosclerosis, inhibiting the entry of viruses into target cells, Acquired Immunodeficiency Syndrome (AIDS), Lupus disorders (such us lupus erythematosus or systemic lupus), erythematosus, Hashimoto's thyroiditis, myasthenia gravis, type I diabetes, nephrotic syndrome, eosinophilia fascitis, hyper IgE syndrome, leprosy (such as lepromatous leprosy), Peridontal disease, Sezary syndrome, idiopathic thrombocytopenia pupura, disorders of the menstrual cycle, glomerulonephritis or cerebral malaria, acute and chronic hepatitis B Virus (HBV) and HCV infection.

The treatment or prevention of these diseases comprises the administration of a therapeutically effective amount of a compound or pharmaceutically acceptable salt or solvate of the compounds of the invention, to a patient in need thereof. Preferably the patient is a warm-blooded animal, more preferably a human.

Preferred diseases are AIDS (HIV-1 or -2 infection), inflammatory and immunoregulatory disorders and diseases including asthma, pulmonary emphysema, allergic diseases and graft rejection as well as autoimmune pathologies such as rheumatoid arthritis, atherosclerosis, psoriasis, systemic lupus erythematosus, ulcerative colitis, multiple sclerosis, glomerulonephritis, together with chronic obstructive pulmonary disease (COPD, including pulmonary fibrosis). Additional fields of application concern certain sort of metastatic cancers and renal diseases.

In a particular preferred embodiment the disease is AIDS (HIV-1 or -2 infection).

The compounds of the present invention are also of value in inhibiting the entry of viruses (such as human immunodeficiency virus (HIV)) into target cells and, therefore, are of value in the prevention of infection by viruses (such as HIV), the treatment of infection by viruses (such as HIV) and the prevention and/or treatment of acquired immune deficiency syndrome (AIDS).

According to a further feature of the present invention there is provided a method for modulating chemokine receptor activity, especially CCR5 receptor activity, in a patient, preferably a warm blooded animal, and even more preferably a human, in need of such treatment, which comprises administering to said animal an effective amount of compound of the present invention, or a pharmaceutically acceptable salt or solvate thereof.

According to one embodiment, the compounds of the invention, their pharmaceutical acceptable salts or solvates may be administered as part of a combination therapy. Thus included within the scope of the present invention are embodiments comprising coadministration of, and compositions and medicaments which contain, in addition to a compound of the present invention, a pharmaceutically acceptable salt or solvate thereof as active ingredient, additional therapeutic agents and/or active ingredients. Such multiple drug regimens, often referred to as combination therapy, may be used in the treatment and prevention of any of the diseases or conditions mediated by or associated with CCR5 chemokine receptor modulation, particularly infection by human immunodeficiency virus, HIV. The use of such combinations of therapeutic agents is especially pertinent with respect to the treatment and prevention of infection and multiplication of the human immunodeficiency virus, HIV, and related pathogenic retroviruses within a patient in need of treatment or one at risk of becoming such a patient. The ability of such retroviral pathogens to evolve within a relatively short period of time into strains resistant to any monotherapy which has been administered to said patient is well known in the literature.

In addition to the requirement of therapeutic efficacy, which may necessitate the use of active agents in addition to the CCR5 chemokine receptor modulating compounds of Formula I or their pharmaceutical acceptable salts or solvates thereof, there may be additional rationales which compel or highly recommend the use of combinations of drugs involving active ingredients which represent adjunct therapy, i.e., which complement and supplement the function performed by the CCR5 chemokine receptor modulating compounds of the present invention. Such supplementary therapeutic agents used for the purpose of auxiliary treatment include drugs which, instead of directly treating or preventing a disease or condition mediated by or associated with CCR5′ chemokine receptor modulation, treat diseases or conditions which directly result from or indirectly accompany the basic or underlying CCR5 chemokine receptor modulated disease or condition. For example, where the basic CCR5 chemokine receptor modulated disease or condition is HIV infection and multiplication, it may be necessary or at least desirable to treat opportunistic infections, neoplasms, and other conditions which occur as the result of the immune-compromised state of the patient being treated. Other active agents may be used with the compounds of Formula I or their pharmaceutical acceptable salts or solvates thereof, e.g., in order to provide immune stimulation or to treat pain and inflammation which accompany the initial and fundamental HIV infection.

Thus, the methods of treatment and pharmaceutical compositions of the present invention may employ the compounds of Formula I or their pharmaceutical acceptable salts or solvates thereof in the form of monotherapy, but said methods and compositions may also be used in the form of multiple therapy in which one or more compounds of Formula I or their pharmaceutically acceptable salts or solvates are coadministered in combination with one or more other therapeutic agents such as those described in detail further herein.

Preferred combinations of the present invention include simultaneous, or sequential treatments with a compound of Formula I, or a pharmaceutical acceptable salt or solvate thereof, and one or more inhibitors of HIV protease and/or inhibitors of HIV reverse transcriptase, preferably selected from the class of non-nucleoside reverse transcriptase inhibitors (NNRTI), including but not limited to nevirapine, delavirdine and efavirenz; from among the nucleoside/nucleotide inhibitors, including but not limited to zidovudine, didanosine, zalcitabine, stavudine, lamivudine, abacavir, adefovir anddipivoxil and from among the protease inhibitors, including but not limited to indinavir, ritonavir, saquinavir, nelfinavir, lopinavir, and amprenavir.

Other agents useful in the above-described preferred embodiment combinations of the present invention include current and to-be-discovered investigational drugs from any of the above classes of inhibitors, including but not limited to FTC, PMPA, fozivudinetidoxil, talviraline, S-1153, MKC-442, MSC-204, MSH-372, DMP450, PNU-140690, ABT-378, KNI-764, TMC120 and TMC125.

There is also included within the scope of the preferred embodiments of the present invention, combinations of a compound of Formula I, or a pharmaceutical acceptable salt or solvate thereof, together with a supplementary therapeutic agent used for the purpose of auxiliary treatment, wherein said supplementary therapeutic agent comprises one or more members independently selected from the group consisting of proliferation inhibitors, e.g., hydroxyurea; immunomodulators, e.g., sargramostim, and various forms of interferon or interferon derivatives; fusion inhibitors, e.g., AMD3100, T-20, T-1249, PRO-140, PRO-542, AD-349, BB-10010 and other chemokine receptor agonists/antagonists; tachykinin receptor modulators, e.g. NK1 antagonists; integrase inhibitors, e.g., AR177; RNaseH inhibitors; inhibitors of viral transcription and RNA replication; and other agents that inhibit viral infection or improve the condition or outcome of HIV-infected individuals through different mechanisms.

Preferred methods of treatment of the present invention for the prevention of HIV infection, or treatment of aviremic and asymptomatic subjects potentially or effectively infected with HIV, include but are not limited to administration of a member independently selected from the group consisting of: (i) a compound within the scope of Formula I or a pharmaceutical acceptable salt or solvate thereof as disclosed herein; (ii) one NNRTI in addition to a compound of (i); (iii) two NRTI in addition to a compound of (i); (iv) one NRTI in addition to the combination of (ii); and (v) a compound selected from the class of protease inhibitors used in place of a NRTI in combinations (iii) and (iv).

The preferred methods of the present invention for therapy of HIV-infected individuals with detectable viremia or abnormally low CD4 counts further include as a member to be selected: (vi) treatment according to (i) above in addition to the standard recommended initial regimens for the therapy of established HIV infections. Such standard regimens include but are not limited to an agent from the class of protease inhibitors in combination with two NRTIs; and (vii) a standard recommended initial regimens for the therapy of established HIV infections, where either the protease inhibitor component, or one or both of the NRTIs is/are replaced by a compound within the scope of Formula I as disclosed herein.

The preferred methods of the present invention for therapy of HIV-infected individuals that have failed antiviral therapy further include as a member to be selected: (viii) treatment according to (i) above, in addition to the standard recommended regimens for the therapy of such patients; and (ix) a standard recommended initial regimens for the therapy of patients who have failed antiretroviral therapy, where either one of the protease inhibitor components, or one or both of the NRTIs is/are replaced by a compound within the scope of Formula I or a pharmaceutical acceptable salt or solvate thereof as disclosed herein.

Additional combinations for use according to the invention include combination of a compound of Formula I, or a pharmaceutical acceptable salt or solvate thereof with another CCR5 modulator, such as a CCR5 agonist; a CCR5 antagonist, such as N-{(1S)-3-[3-(3-isopropyl-5-methyl-4H-1,2,4-triazol-4-yl)-exo-8-azabicyclo[3.2.1]oct-8-yl]-1-phenylpropyl}-4,4-difluorocyclohexanecarboxamide; a CCR1 antagonist, such as BX-471; a beta adrenoceptor agonist, such as salmeterol; a corticosteroid agonist, such fluticasone propionate; a LTD4 antagonist, such asmontelukast; a muscarinic antagonist, such as tiotropium bromide; a PDE4 inhibitor, such ascilomilast or roflumilast; a COX-2 inhibitor, such ascelecoxib, valdecoxib or rofecoxib; an alpha-2-delta ligand, such as gabapentin or pregabalin; a beta-interferon, such as REBIF; a TNF receptor modulator, such as aTNF-alpha inhibitor (e.g. adalimumab); a HMG CoA reductase inhibitor, such as a statin (e.g. atorvastatin); or an immunosuppressant, such as cyclosporine; or a macrolide such as tacrolimus.

In the above-described preferred embodiment combinations of the present invention, the compound of formula I, a pharmaceutically acceptable salt or solvate thereof and other therapeutic active agents may be administered in terms of dosage forms either separately or in conjunction with each other, and in terms of their time of administration, either serially or simultaneously. Thus, the administration of one component agent may be prior to, concurrent with, or subsequent to the administration of the other component agent(s).

The invention also provides pharmaceutical compositions comprising a compound of formula I or a pharmaceutically acceptable salt or solvate thereof and at least one pharmaceutically acceptable carrier, diluent, excipient and/or adjuvant. As indicated above, the invention also covers pharmaceutical compositions which contain, in addition to a compound of the present invention, a pharmaceutically acceptable salt or solvate thereof as active ingredient, additional therapeutic agents and/or active ingredients.

Another object of this invention is a medicament comprising at least one compound of the invention, or a pharmaceutically acceptable salt or solvate thereof, as active ingredient.

The invention also provides the use of a compound of formula I or a pharmaceutically acceptable salt or solvate thereof for the manufacture of a medicament. Preferably, the medicament is used for the treatment or prevention of autoimmune, inflammatory, infectious, proliferative or hyperproliferative diseases, or immunologically mediated diseases (including rejection of transplanted organs or tissues and Acquired Immunodeficiency Syndrome (AIDS)); examples of these conditions are:
(1) (the respiratory tract) obstructive diseases of airways including: chronic obstructive pulmonary disease (COPD) (such as irreversible COPD); pulmonary fibrosis; asthma {such as bronchial, allergic, intrinsic, extrinsic or dust asthma, particularly chronic or inveterate asthma (for example late asthma or airways hyper-responsiveness)}; bronchitis {such as eosinophilic bronchitis}; acute, allergic, atrophic rhinitis or chronic rhinitis including rhinitis caseosa, hypertonic rhinitis, rhinitis purulenta, rhinitis sicca or rhinitis medicamentosa; membranous rhinitis including croupous, fibrinous or pseudomembranous rhinitis or scrofoulous rhinitis; seasonal rhinitis including rhinitis nervosa (hay fever) or vasomotor rhinitis; sarcoidosis; farmer's lung and related diseases; nasal polyposis; fibroid lung or idiopathic interstitial pneumonia;
(2) (bone and joints) arthrides including rheumatic, infectious, autoimmune, seronegative spondyloarthropathies (such as ankylosing spondylitis, psoriatic arthritis or Reiter's disease), Behçet's disease, Sjogren's syndrome or systematic sclerosis;
(3) (skin and eyes) psoriasis, atopic dermatitis, contact dermatitis or other eczematous dermitides, seborrhoetic dermatitis, Lichen planus, Phemphigus, bullous Phemphigus, Epidermolysis bullosa, urticaria, angiodermas, vasiculitides erythermas, cutaneaous eosinophilias, uveitis, Alopecia greata or vernal conjunctivitis;
(4) (gastrointestinal tract) Coeliac disease, proctitis, eosinophilic gastro-enteritis, mastocytosis, Crohn's disease, ulcerative colitis, irritable bowel disease or food-related allergies which have effects remote from the gut (for example migraine, rhinitis or eczema);
(5) (Allorgraft rejection) acute and chronic following, for example, transplantation of kidney, heart, liver, lung bone marrow, skin or cornea; or chronic graft versus host disease; and/or
(6). (other tissues or diseases) Alzheimer's disease, multiple sclerosis, atherosclerosis, inhibiting the entry of viruses into target cells, Acquired Immunodeficiency Syndrome (AIDS), Lupus disorders (such us lupus erythematosus or systemic lupus), erythematosus, Hashimoto's thyroiditis, myasthenia gravis, type I diabetes, nephrotic syndrome, eosinophilia fascitis, hyper IgE syndrome, leprosy (such as lepromatous leprosy), Peridontal disease, Sezary syndrome, idiopathic thrombocytopenia pupura, disorders of the menstrual cycle, glomerulonephritis or cerebral malaria, acute and chronic hepatitis B Virus (HBV) and HCV infection,

Preferred diseseases are AIDS (HIV-1 or -2 infection), inflammatory and immunoregulatory disorders and diseases including asthma, pulmonary emphysema, allergic diseases and graft rejection as well as autoimmune pathologies such as rheumatoid arthritis, atherosclerosis, psoriasis, systemic lupus erythematosus, ulcerative colitis, multiple sclerosis, glomerulonephritis, together with chronic obstructive pulmonary disease (COPD, including pulmonary fibrosis). Additional fields of application concern certain sort of metastatic cancers and renal diseases.

In a particular preferred embodiment the disease is AIDS (HIV-1 or -2 infection).

The invention also provides the use of a compound of formula I or a pharmaceutically acceptable salt or solvate thereof for the manufacture of a medicament for inhibiting the entry of viruses (such as human immunodeficiency virus (HIV)) into target cells and, therefore, for the prevention of infection by viruses (such as HIV), the treatment of infection by viruses (such as HIV) and the prevention and/or treatment of acquired immune deficiency syndrome (AIDS).

According to a further feature of the present invention there is provided the use of a compound of formula I or a pharmaceutically acceptable salt or solvate thereof for the manufacture of a medicament for modulating chemokine receptor activity, especially CCR5 receptor activity, in a patient, in need of such treatment, which comprises administering to said patient an effective amount of compound of the present invention, or a pharmaceutically acceptable salt or solvate thereof.

Preferably, the patient is a warm-blooded animal, more preferably a human.

As set forth above, the compounds of the invention, their pharmaceutically acceptable salts or solvates may be used in monotherapy or in combination therapy, such as bi- or tritherapy. Thus, according to one embodiment, the invention provides the use of a compound of the invention for the manufacture of a medicament for at least one of the purposes described above, wherein said medicament is administered to a patient in need thereof, preferably a warm-blooded animal, and even more preferably a human, in combination with at least one additional therapeutic agent and/or active ingredient. The benefits and advantages of such a multiple drug regimen, possible administration regimens as well as suitable additional therapeutic agents and/or active ingredients are those described above.

Generally, for pharmaceutical use, the compounds of the inventions may be formulated as a pharmaceutical preparation comprising at least one compound of the invention and at least one pharmaceutically acceptable carrier, diluent, excipient and/or adjuvant, and optionally one or more further pharmaceutically active compounds.

By means of non-limiting examples, such a formulation may be in a form suitable for oral administration, for parenteral administration (such as by intravenous, intramuscular or subcutaneous injection or intravenous infusion), for topical administration (including ocular), for administration by inhalation, by a skin patch, by an implant, by a suppository, etc. Such suitable administration forms—which may be solid, semi-solid or liquid, depending on the manner of administration—as well as methods and carriers, diluents and excipients for use in the preparation thereof, will be clear to the skilled person; reference is made to the latest edition of Remington's Pharmaceutical Sciences.

Some preferred, but non-limiting examples of such preparations include tablets, pills, powders, lozenges, sachets, cachets, elixirs, suspensions, emulsions, solutions, syrups, aerosols, ointments, cremes, lotions, soft and hard gelatin capsules, suppositories, drops, sterile injectable solutions and sterile packaged powders (which are usually reconstituted prior to use) for administration as a bolus and/or for continuous administration, which may be formulated with carriers, excipients, and diluents that are suitable per se for such formulations, such as lactose, dextrose, sucrose, sorbitol, mannitol, starches, gum acacia, calcium phosphate, alginates, tragacanth, gelatin, calcium silicate, microcrystalline cellulose, polyvinylpyrrolidone, polyethylene glycol, cellulose, (sterile) water, methylcellulose, methyl- and propylhydroxybenzoates, talc, magnesium stearate, edible oils, vegetable oils and mineral oils or suitable mixtures thereof. The formulations can optionally contain other substances that are commonly used in pharmaceutical formulations, such as lubricating agents, wetting agents, emulsifying and suspending agents, dispersing agents, desintegrants, bulking agents, fillers, preserving agents, sweetening agents, flavoring agents, flow regulators, release agents, etc. The compositions may also be formulated so as to provide rapid, sustained or delayed release of the active compound(s) contained therein.

The pharmaceutical preparations of the invention are preferably in a unit dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use. Generally, such unit dosages will contain between 0.05 and 1000 mg, and usually between 1 and 500 mg, of the at least one compound of the invention, e.g. about 10, 25, 50, 100, 200, 300 or 400 mg per unit dosage.

Usually, depending on the condition to be prevented or treated and the route of administration, the active compound of the invention will usually be administered between 0.01 to 100 mg per kilogram, more often between 0.1 and 50 mg, such as between 1 and 25 mg, for example about 0.5, 1, 5, 10, 15, 20 or 25 mg, per kilogram body weight day of the patient per day, which may be administered as a single daily dose, divided over one or more daily doses, or essentially continuously, e.g. using a drip infusion.

### DEFINITIONS

The definitions and explanations below are for the terms as used throughout the entire application, including both the specification and the claims.

When describing the compounds of the invention, the terms used are to be construed in accordance with the following definitions, unless indicated otherwise.

Where groups may be substituted, such groups may be substituted with one or more substituents, and preferably with one, two or three substituents.

Substituents may be selected from but not limited to, for example, the group comprising halogen, hydroxyl, oxo, nitro, amido, carboxy, amino, cyano haloalkoxy, and haloalkyl.

As used herein the terms such as “alkyl, aryl, or cycloalkyl, each being optionally substituted with . . . ” or “alkyl, aryl, or cycloalkyl, optionally substituted with . . . ” encompasses “alkyl optionally substituted with . . . ”, “aryl optionally substituted with . . . ” and “cycloalkyl optionally substituted with . . . ”.

The term “halo” or “halogen” means fluoro, chloro, bromo, or iodo.

The term “alkyl” by itself or as part of another substituent refers to a hydrocarbyl radical of Formula CnH₂H₂ₙ₊₁ wherein n is a number greater than or equal to 1. Generally, alkyl groups of this invention comprise from 1 to 6 carbon atoms, preferably from 1 to 4 carbon atoms, more preferably from 1 to 3 carbon atoms, still more preferably 1 to 2 carbon atoms. Alkyl groups may be linear or branched and may be substituted as indicated herein.

Suitable alkyl groups include methyl, ethyl, n-propyl, i-propyl, n-butyl, i-butyl, s-butyl and t-butyl, pentyl and its isomers (e.g. n-pentyl, iso-pentyl), and hexyl and its isomers (e.g. n-hexyl, iso-hexyl).

The term “hydroxyalkyl” includes but is not limited to hydroxymethyl, 1-hydroxyethyl, 2-hydroxyethyl, 1-hydroxypropyl, 2-hydroxypropyl, 2-hydroxy-2-methylethyl, 1-hydroxypropyl, 2-hydroxypropyl, 3-hydroxypropyl, 1-hydroxybutyl, 2-hydroxybutyl, 3-hydroxybutyl, 4-hydroxybutyl, 2-hydroxy-2-methylpropyl, 1-(hydroxymethyl)-2-methylpropyl, 1,1-dimethyl-2-hydroxyethyl, 5-hydroxypentyl, 2-methyl-3-hydroxypropyl, 3,4-dihydroxybutyl, and so forth “Alkoxyalkyl”, refers to an alkyl group substituted with one to two R^{b}, wherein R^{b} is alkoxy as defined below. For example heterocyclylalkyl refers to an alkyl group substituted with one to two R^{f}, wherein R^{f} is heterocyclyl as defined below. For example, “aralkyl”, or “arylalkyl” refers to a substituted alkyl group as defined above wherein at least one of the alkyl substituents is an aryl as defined below, such as benzyl. For example, “heteroarylalkyl” refers to a substituted alkyl group as defined above, wherein at least one of the alkyl substituents is a heteroaryl as defined below, such as pyridinyl.

The term “haloalkyl” alone or in combination, refers to an alkyl radical having the meaning as defined above wherein one or more hydrogens are replaced with a halogen as defined above. Non-limiting examples of such haloalkyl radicals include chloromethyl, 1-bromoethyl, fluoromethyl, difluoromethyl, trifluoromethyl, 1,1,1-trifluoroethyl and the like.

The term “cycloalkyl” as used herein is a cyclic alkyl group, that is to say, a monovalent, saturated, or unsaturated hydrocarbyl group having 1 or 2 cyclic structures. Cycloalkyl includes monocyclic or bicyclic hydrocarbyl groups. Cycloalkyl groups may comprise 3 or more carbon atoms in the ring and generally, according to this invention comprise from 3 to 10, more preferably from 3 to 8 carbon atoms still more preferably from 3 to 6 carbon atoms. Examples of cycloalkyl groups include but are not limited to cyclopropyl, cyclobutyl, cyclopentyl, cyclohexyl, with cyclopropyl being particularly preferred. An “optionally substituted cycloalkyl” refers to a cycloalkyl having optionally one or more substituent(s) (for example 1 to 3 substituent(s), for example 1, 2 or 3 substituent(s)), selected from those defined above for substituted alkyl. When the suffix “ene” is used in conjunction with a cyclic group, this is intended to mean the cyclic group as defined herein having two single bonds as points of attachment to other groups.

The term “cycloalkylalkyl” includes but is not limited to cyclopropylmethyl, cyclobutylmethyl, cyclopentylmethyl, cyclohexylmethyl, 1-cyclopentylethyl, 1-cyclohexylethyl, 2-cyclopentylethyl, 2-cyclohexylethyl, cyclobutylpropyl, cyclopentylpropyl, 3-cyclopentylbutyl, cyclohexylbutyl and the like.

The term “alkenyl” as used herein refers to an unsaturated hydrocarbyl group, which may be linear or branched, comprising one or more carbon-carbon double bonds. Suitable alkenyl groups comprise between 2 and 6 carbon atoms, preferably between 2 and 4 carbon atoms, still more preferably between 2 and 3 carbon atoms. Examples of alkenyl groups are ethenyl, 2-propenyl, 2-butenyl, 3-butenyl, 2-pentenyl and its isomers, 2-hexenyl and its isomers, 2,4-pentadienyl and the like. The term “alkynyl” as used herein refers to a class of monovalent unsaturated hydrocarbyl groups, wherein the unsaturation arises from the presence of one or more carbon-carbon triple bonds. Alkynyl groups typically, and preferably, have the same number of carbon atoms as described above in relation to alkenyl groups. Non limiting examples of alkynyl groups are ethynyl, 2-propynyl, 2-butynyl, 3-butynyl, 2-pentynyl and its isomers, 2-hexynyl and its isomers—and the like. The term “alkylene” includes methylene, ethylene, methylmethylene, propylene, ethylethylene, and 1,2-dimethylethylene. “Cycloalkylene” herein refers to a saturated homocyclic hydrocarbyl biradical of Formula CₙH₂ₙ₋₂. Cycloalkylene groups of this invention preferably comprise the same number of carbon atoms as their cycloalkyl radical counterparts.

Suitable cycloalkylene groups are C₃₋₆ cycloalkylene group, preferably a C₃₋₅ cycloalkylene (i.e. 1,3-cyclopropylene, 1,1-cyclopropylene, 1,1-cyclobutylene, 1,2-cyclobutylene, 1,3-cyclopentylene, or 1,1-cyclopentylene), more preferably a C₃₋₄ cycloalkylene (i.e. 1,3-cyclopropylene, 1,1-cyclopropylene, 1,1-cyclobutylene, 1,2-cyclobutylene).

The terms “heterocyclyl” or “heterocyclo” as used herein by itself or as part of another group refer to non-aromatic, fully saturated or partially unsaturated cyclic groups (for example, 3 to 7 member monocyclic, 7 to 11 member bicyclic, or containing a total of 3 to 10 ring atoms) which have at least one heteroatom in at least one carbon atom-containing ring. Each ring of the heterocyclic group containing a heteroatom may have 1, 2, 3 or 4 heteroatoms selected from nitrogen, oxygen and/or sulfur atoms, where the nitrogen and sulfur heteroatoms may optionally be oxidized and the nitrogen heteroatoms may optionally be quaternized. The heterocyclic group may be attached at any heteroatom or carbon atom of the ring or ring system, where valence allows. The rings of multi-ring heterocycles may be fused, bridged and/or joined through one or more spiro atoms. Non limiting exemplary heterocyclic groups include aziridinyl, oxiranyl, thiiranyl, piperidinyl, azetidinyl, 2-imidazolinyl, pyrazolidinyl imidazolidinyl, isoxazolinyl, oxazolidinyl, isoxazolidinyl, thiazolidinyl, isothiazolidinyl, piperidinyl, succinimidyl, 3H-indolyl, indolinyl, isoindolinyl, 2H-pyrrolyl, 1-pyrrolinyl, 2-pyrrolinyl, 3-pyrrolinyl, pyrrolidinyl, 4H-quinolizinyl, 2-oxopiperazinyl, piperazinyl, homopiperazinyl, 2-pyrazolinyl, 3-pyrazolinyl, tetrahydro-2H-pyranyl, 2H-pyranyl, 4H-pyranyl, 3,4-dihydro-2H-pyranyl, oxetanyl, thietanyl, 3-dioxolanyl, 1,4-dioxanyl, 2,5-dioximidazolidinyl, 2-oxopiperidinyl, 2-oxopyrrolodinyl, indolinyl, tetrahydropyranyl, tetrahydrofuranyl, tetrahydrothiophenyl, tetrahydroquinolinyl, tetrahydroisoquinolin-1-yl, tetrahydroisoquinolin-2-yl, tetrahydroisoquinolin-3-yl, tetrahydroisoquinolin-4-yl, thiomorpholin-4-yl, thiomorpholin-4-ylsulfoxide, thiomorpholin-4-ylsulfone, 1,3-dioxolanyl, 1,4-oxathianyl, 1,4-dithianyl, 1,3,5-trioxanyl, 1H-pyrrolizinyl, tetrahydro-1,1-dioxothiophenyl, N-formylpiperazinyl, and morpholin-4-yl.

The term “aryl” as used herein refers to a polyunsaturated, aromatic hydrocarbyl group having a single ring (i.e. phenyl) or multiple aromatic rings fused together (e.g. naphtyl). or linked covalently, typically containing 5 to 12 atoms; preferably 6 to 10, wherein at least one ring is aromatic. The aromatic ring may optionally include one to two additional rings (either cycloalkyl, heterocyclyl or heteroaryl) fused thereto. Aryl is also intended to include the partially hydrogenated derivatives of the carbocyclic systems enumerated herein. Non-limiting examples of aryl comprise phenyl, biphenylyl, biphenylenyl, 5- or 6-tetralinyl, 1-, 2-, 3-, 4-, 5-, 6-, 7- or 8-azulenyl, naphthalen-1- or -2-yl, 4-, 5-, 6 or 7-indenyl, 1-2-, 3-, 4- or 5-acenaphtylenyl, 3-, 4- or 5-acenaphtenyl, 1-, 2-, 3-, 4- or 10-phenanthryl, 1- or 2-pentalenyl, 4- or 5-indanyl, 5-, 6-, 7- or 8-tetrahydronaphthyl, 1,2,3,4-tetrahydronaphthyl, 1,4-dihydronaphthyl, 1-, 2-, 3-, 4- or 5-pyrenyl.

The term “arylene” as used herein is intended to include divalent carbocyclic aromatic ring systems such as phenylene, biphenylylene, naphthylene, indenylene, pentalenylene, azulenylene and the like. Arylene is also intended to include the partially hydrogenated derivatives of the carbocyclic systems enumerated above. Non-limiting examples of such partially hydrogenated derivatives are 1,2,3,4-tetrahydronaphthylene, 1,4-dihydronaphthylene and the like.

Where at least one carbon atom in an aryl group is replaced with a heteroatom, the resultant ring is referred to herein as a heteroaryl ring.

The term “heteroaryl” or “aromatic heterocycle” as used herein by itself or as part of another group refers but is not limited to 5 to 12 carbon-atom aromatic rings or ring systems containing 1 to 2 rings which are fused together or linked covalently, typically containing 5 to 6 atoms; at least one of which is aromatic, in which one or more carbon atoms in one or more of these rings is replaced by oxygen, nitrogen and/or sulfur atoms where the nitrogen and sulfur heteroatoms may optionally be oxidized and the nitrogen heteroatoms may optionally be quaternized. Such rings may be fused to an aryl, cycloalkyl, heteroaryl or heterocyclyl ring. Non-limiting examples of such heteroaryl, include: pyrrolyl, furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, oxazinyl, dioxinyl, thiazinyl, triazinyl, imidazo[2,1-b][1,3]thiazolyl, thieno[3,2-b]furanyl, thieno[3,2-b]thiophenyl, thieno[2,3-d][1,3]thiazolyl, thieno[2,3-d]imidazolyl, tetrazolo[1,5-a]pyridinyl, indolyl, indolizinyl, isoindolyl, benzofuranyl, isobenzofuranyl, benzothiophenyl, isobenzothiophenyl, indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1,2,3-benzothiadiazolyl, 2,1,3-benzothiadiazolyl, thienopyridinyl, purinyl, imidazo-[1,2-a]pyridinyl, 6-oxo-pyridazin-1(6H)-yl, 2-oxopyridin-1(2H)-yl, 6-oxo-pyridazin-1(6H)-yl, 2-oxopyridin-1(2H)-yl, 1,3-benzodioxolyl, quinolinyl, isoquinolinyl, cinnolinyl, quinazolinyl, quinoxalinyl.

The bonds from an asymmetric carbon in compounds of the present invention may be depicted herein using a solid line [symbol] a zigzag line [symbol] a solid wedge [symbol] or a dotted wedge [symbol] The use of either a solid or dotted wedge to depict bonds from an asymmetric carbon atoms is meant to indicate that only the stereoisomer shown is meant to be included.

The compounds of the invention may also contain more than one asymmetric carbon atom. In those compounds, the use of a solid line to depict bonds from asymmetric carbon atoms is meant to indicate that all possible stereoisomers are meant to be included, unless it is clear from the context that a specific stereoisomer is intended.

The compounds of the invention may be in the form of pharmaceutically acceptable salts. Pharmaceutically acceptable salts of the compounds of formula I include the acid addition and base salts thereof. Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts. Preferred, pharmaceutically acceptable salts include hydrochloride/chloride, hydrobromide/bromide, bisulphate/sulphate, nitrate, citrate, and acetate.

When the compounds of the invention contain an acidic group as well as a basic group the compounds of the invention may also form internal salts, and such compounds are within the scope of the invention. When the compounds of the invention contain a hydrogen-donating heteroatom (e.g. NH), the invention also covers salts and/or isomers formed by transfer of said hydrogen atom to a basic group or atom within the molecule.

Pharmaceutically acceptable salts of compounds of Formula I may be prepared by one or more of these methods:

(i) by reacting the compound of Formula I with the desired acid;

(ii) by reacting the compound of Formula I with the desired base;

(iii) by removing an acid- or base-labile protecting group from a suitable precursor of the compound of Formula I or by ring-opening a suitable cyclic precursor, for example, a lactone or lactam, using the desired acid; or

(iv) by converting one salt of the compound of Formula I to another by reaction with an appropriate acid or by means of a suitable ion exchange column.

All these reactions are typically carried out in solution. The salt, may precipitate from solution and be collected by filtration or may be recovered by evaporation of the solvent. The degree of ionization in the salt may vary from completely ionized to almost non-ionized.

The term ‘solvate’ is used herein to describe a molecular complex comprising the compound of the invention and one or more pharmaceutically acceptable solvent molecules, for example, ethanol. The term ‘hydrate’ is employed when said solvent is water.

All references to compounds of formula I include references to salts, solvates, multi-component complexes and liquid crystals thereof and to solvates, multi-component complexes and liquid crystals of salts thereof.

The compounds of the invention include compounds of formula I as hereinbefore defined, including all polymorphs and crystal habits thereof, prodrugs and isomers thereof (including optical, geometric and tautomeric isomers) and isotopically-labeled compounds of formula I.

In addition, although generally, with respect to the salts of the compounds of the invention, pharmaceutically acceptable salts are preferred, it should be noted that the invention in its broadest sense also include non-pharmaceutically acceptable salts, which may for example be used in the isolation and/or purification of the compounds of the invention. For example, salts formed with optically active acids or bases may be used to form diastereoisomeric salts that can facilitate the separation of optically active isomers of the compounds of Formula I above.

The invention also generally covers all pharmaceutically acceptable prodrugs and prodrugs of the compounds of Formula I.

The term “pro-drug” as used herein means the pharmacologically acceptable derivatives such as esters, amides and phosphates, such that the resulting in vivo biotransformation product of the derivative is the active drug. Pro-drugs are characterized by increased bio-availability and are readily metabolized into the active inhibitors in vivo. The term “pre-drug”, as used herein, means any compound that will be modified to form a drug species, wherein the modification may take place either inside or outside of the body, and either before or after the pre-drug reaches the area of the body where administration of the drug is indicated.

The term “patient” refers to a warm-blooded animal, more preferably a human, who/which is awaiting or receiving medical care or is or will be the object of a medical procedure.

The term “human” refers to subject of both genders and at any stage of development (i.e. neonate, infant, juvenile, adolescent, adult).

The term “transplant” refers to the grafting, implantation or transplantation of organs, tissues, cells (e.g., bone marrow) and/or biocompatible materials onto or into the body of an animal. The term encompasses the transfer of tissues from one part of the animal's body to another part and the transfer of organs, tissues, and/or cells obtained from a donor animal (either directly or indirectly such as an organ or tissue produced in vitro by culturing cells obtained from the animal) into a recipient animal. The animal is suitably a warm-blooded vertebrate, is typically a mammal, and is especially a primate (e.g. a human). The term “transplant rejection” means any immune reaction in the recipient directed against grafted organs, tissues, cells, and/or biocompatible materials.

The term “therapeutically effective amount” (or more simply an “effective amount”) as used herein means the amount of active agent or active ingredient (e.g. chemokine receptor CCR5 modulator, i.e. a CCR5 agonist or a CCR5 antagonist, especially a CCR5 agonist) which is sufficient to achieve the desired therapeutic or prophylactic effect in the individual to which it is administered.

The term “administration”, or a variant thereof (e.g., “administering”), means providing the active agent or active ingredient (e.g., a CCR5 modulator), alone or as part of a pharmaceutically acceptable composition, to the patient in whom/which the condition, symptom, or disease is to be treated or prevented.

By “pharmaceutically acceptable” is meant that the ingredients of a pharmaceutical composition are compatible with each other and not deleterious to the recipient thereof.

The term “agonist” as used herein means a ligand that activates an intracellular response when it binds to a receptor. An agonist according to the invention may promote internalization of a cell surface receptor such that the cell surface concentration of a receptor is decreased or remove.

The term “antagonist” as used herein means a ligand which competitively binds to a receptor at the same site as an agonist, but does not activate an intracellular response initiated by an active form of the receptor. An antagonist thereby inhibits the intracellular response induced by an agonist.

The term “pharmaceutical vehicle” as used herein means a carrier or inert medium used as solvent or diluent in which the pharmaceutically active agent is formulated and/or administered. Non-limiting examples of pharmaceutical vehicles include creams, gels, lotions, solutions, liposomes.

The present invention will be better understood with reference to the following examples. These examples are intended to representative of specific embodiments of the invention, and are not intended as limiting the scope of the invention.

### EXAMPLES

### Chemistry Examples

All temperatures are expressed in QC and all reactions were carried out at room temperature unless otherwise stated.

Analytical thin layer chromatography (TLC) was used to monitor reactions, establish flash chromatography conditions and verify purity of intermediates or final products. TLC plates used were Merck TLC aluminium sheet silica gel 60 F₂₅₄ purchased from VWR International. TLC plates were revealed using ultraviolet irradiation (wavelength=254 nm) at room temperature or bromocresol green spray reagent at 0.1% in propan-2-ol purchased from VWR International upon heating at 160° C. or KMnO₄ revelator upon heating at 160° C. The KMnO₄ revelator was prepared by dissolving 3 g of potassium permanganate, 20 g of sodium carbonate, 0.5 g of sodium hydroxide in 100 mL of distilled water.

HPLC-MS spectra were obtained on Waters instruments using Electropsray ionization (ESI). Samples are injected by a Waters 2767 sample manager. A Waters 2525 binary pump module is linked to a Waters 2996 photodiode array detector and a Waters micromass ZQ-2000. The column used is a Sunfire C18 5μ; 4.6*50 mm. Eluent is a mixture of solution A (0.1% TFA in H₂O) and solution B (0.1% TFA in ACN): 5% solution B for 1 min, gradient from 5% solution B to 95% solution B over 4 min, 95% solution B for 0.2 min and 5% solution B for 0.8 min.

¹H and ¹³C NMR spectra were recorded on a Bruker 300 MHz. Chemical shifts are expressed in parts per million, (ppm, δ units). Coupling constants are expressed in Hertz units (Hz). Splitting patterns describe apparent multiplicities and are described as s (singlet), d (doublet), t (triplet), q (quartet), m (multiplet), or br (broad).

Solvents, reagents and starting materials were purchased from well known chemical suppliers such as for example Sigma Aldrich, Acros Organics, Eurisotop, VWR International, Sopachem and Polymer labs and the following abbrviations are used

### ACN: Acetonitrile,

### DCM: Dichloromethane,

### DMF: N,N-dimethylformamide,

EtOAc: Ethyl acetate,

### EtOH: Ethanol,

HOBt: 1-hydroxybenzotriazole,
MeOH: Methanol,
   RT: Room temperature,

### TEA: Triethylamine,

TBTU: O-(1H-Benzotriazol-1-yl)-N,N,N′,N′-tetramethyluronium tetrafluoroborate,

### Y: Yield,

### g: Grams,

### mg: Milligrams,

### L: Liters,

### mL: Milliliters,

### μL: Microliters,

### mol: Moles,

mmol: Millimoles,

### h: Hours,

### min: Minutes,

TLC: Thin layer chromatography,
MW: Molecular weight,

### eq: Equivalent,

μwave: Microwave,

### THF: Tetrahydrofuran,

TFA: Trifluoroacetic acid,

### Ac: Acetyl,

EDC: 1-(3-dimethylaminopropyl)-3-ethylcarbodiimide hydrochloride,
DABCO: 1,4-diazabicyclo[2,2,2]octane,
n-BuLi: n-butyl Lithium.

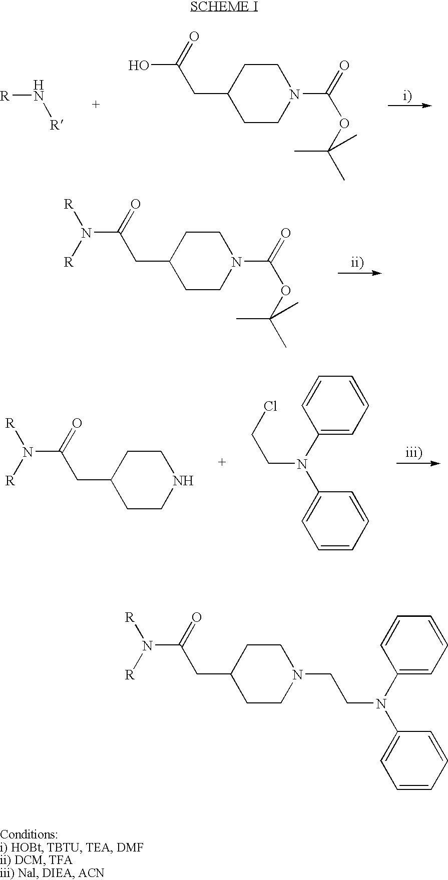

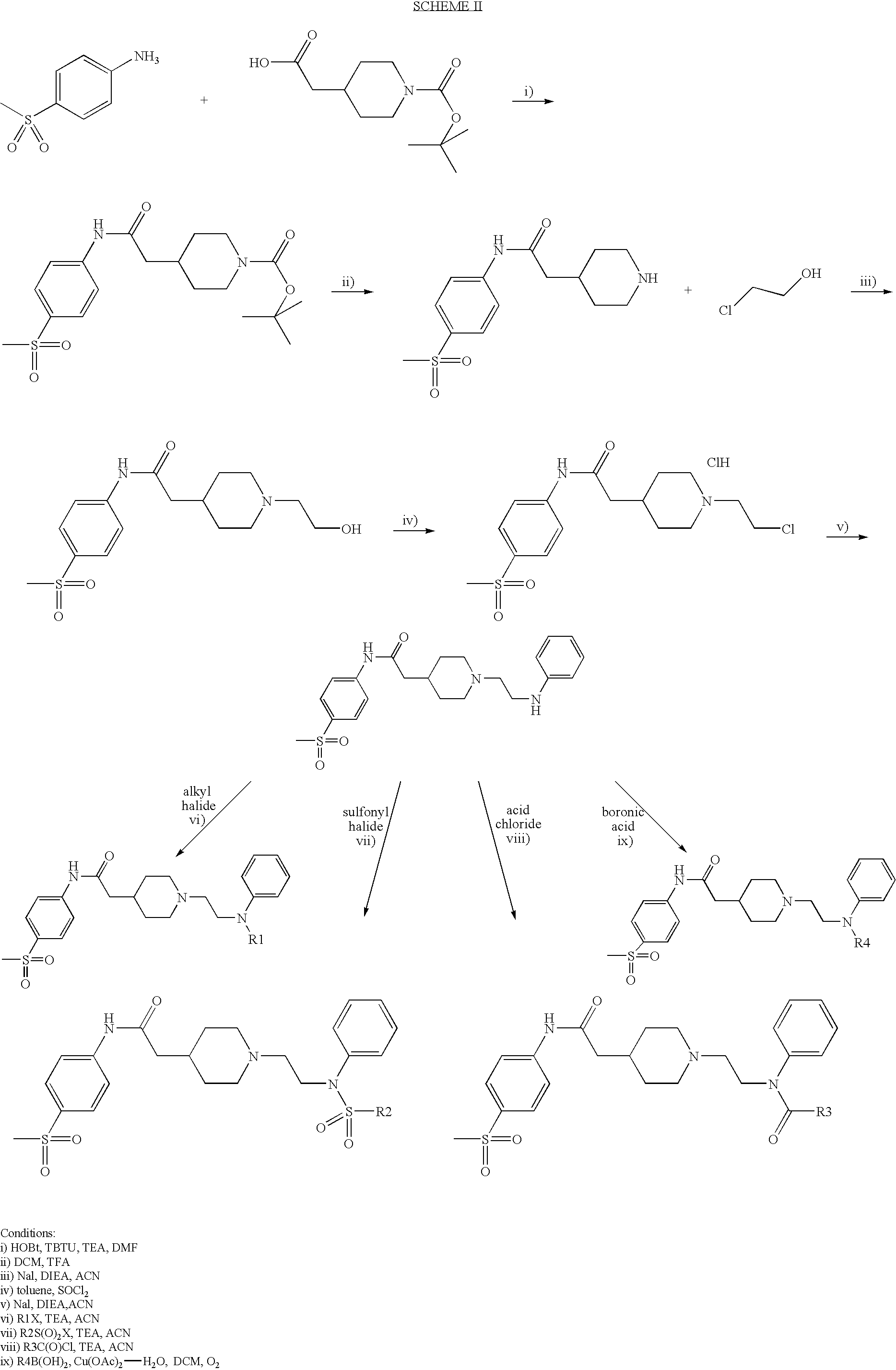

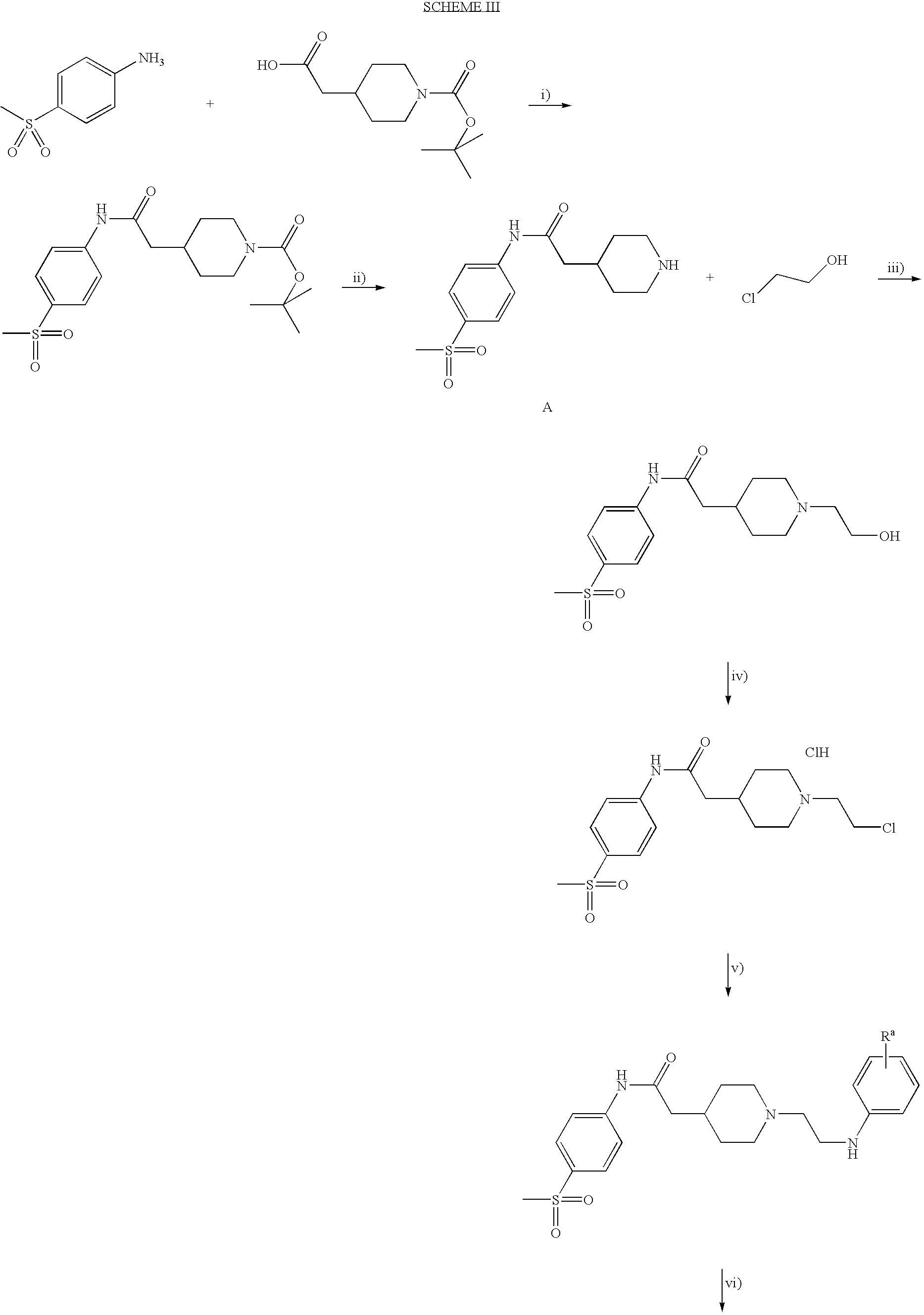

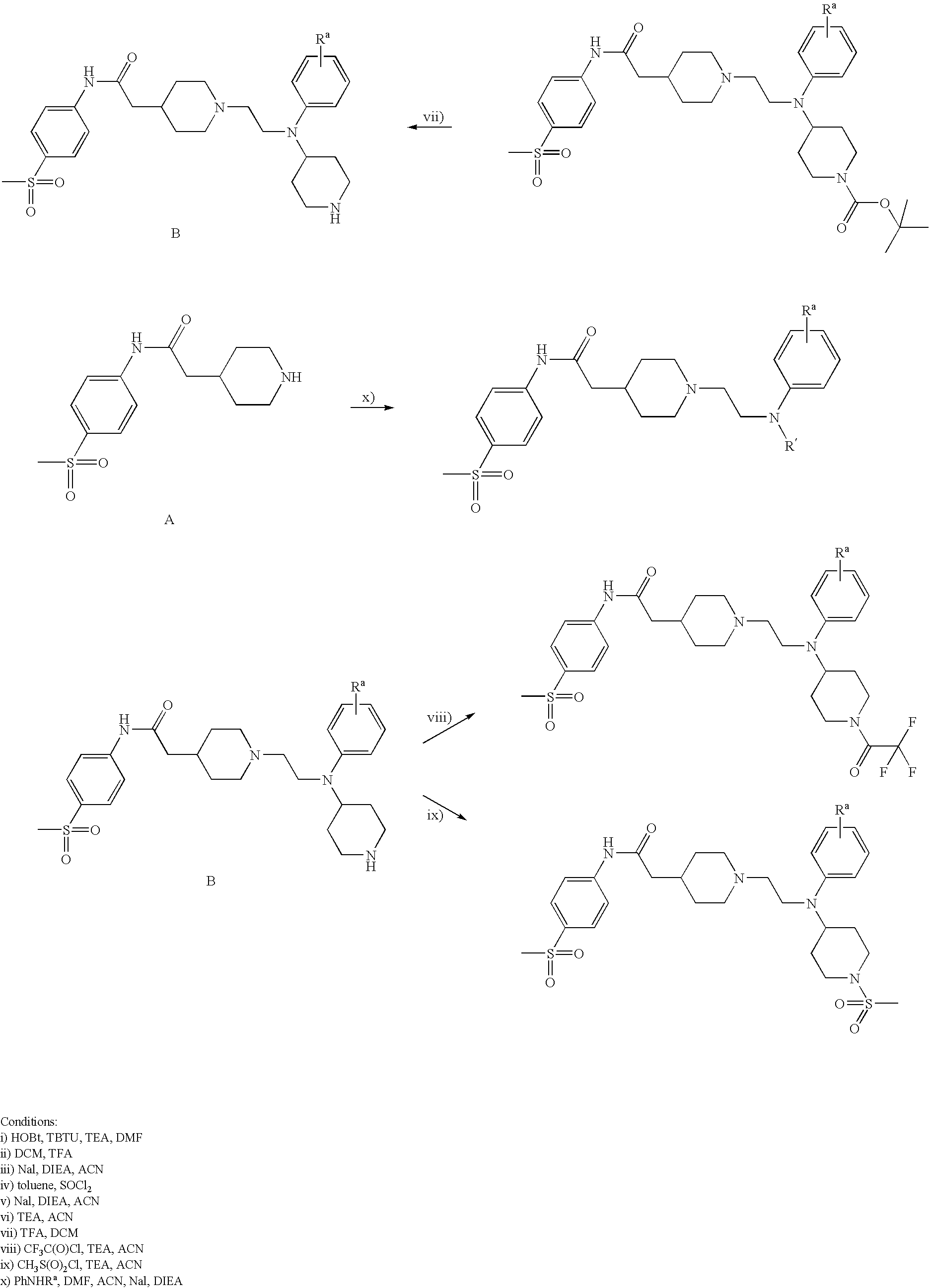

### Synthesis of Intermediate 1: [1-(2-diphenylamino-ethyl)-piperidin-4-yl]-acetic acid methyl ester

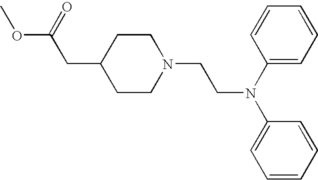

To a solution of piperidine-4-acetic acid methyl ester

(162.8 mg; 1.04 mmol), in ACN (4 ml) was added (2-Chloro-ethyl)-diphenyl-amine (J. Med. Chem. 1992, 35, 1042-1049) (240 mg; 1.04 mmol), tetrabutylammonium iodide (38.4 mg; 0.11 mmol) and potassium carbonate (430 mg; 3.11 mmol). The mixture was refluxed during 3 days. The reaction mixture was cooled and filtered over silica gel and concentrated. The residue was purified by silica gel chromatography (eluent:DCM and MeOH/DCM: 5/95) to afford after dry evaporation the title intermediate as an oil (128 mg; Y:35%).

MS: (M+H)⁺=353.

### Synthesis of Intermediate 2: 1-(2-diphenylamino-ethyl)-piperidine-4-acetic acid dihydrochlorid salt

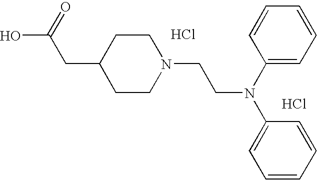

The resin Amberlyst® A26(OH) (1.3 g) was added to a solution of 1-(2-diphenylamino-ethyl)-piperidine-4-acetic acid methyl ester (128 mg; 0.36 mmol) in MeOH (2 ml). The reaction mixture was stirred at RT overnight. The mixture was filtered and washed 3 times with MeOH and 3 times with ACN. The resin was added to a solution of ACN (1 ml) and aqueous 1 M HCl (4 ml). The mixture was stirred for 3 hours. The mixture was filtered and washed 3 times with ACN. The residue was evaporated to give the title intermediate as an oil (87 mg; Y: 73%).

MS: (M+H)⁺=339.

### General Method A:

A solution of HOBt (282 mg; 0.88 mmol) and TBTU (135 mg; 0.88 mmol) in DMF (1 ml) was added to a solution of 1-BOC-piperidine-4-ylacetic acid (200 mg; 0.8 mmol) and the corresponding aniline (0.8 mmol) in DMF (1 ml) followed by TEA (300 μL; 2.2 mmol). The reaction mixtures were stirred 1 hour at RT and 2 hours at 50° C. The resulting mixtures were evaporated under vacuum, dissolved in DCM and washed with an aqueous solution of NaHCO₃ and water. The organic phases were transferred to an ISOLUTE® PE-AX column and then to an ISOLUTE® SCX2 column using MeOH as eluent, to afford after dry evaporation under reduced pressure the desired intermediates (table 3).

TABLE 3

| Intermediate | Structure |
|---|---|
| 3 | 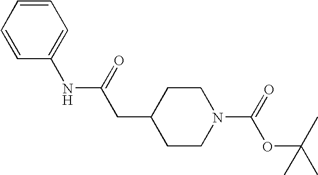 |
| 4 | 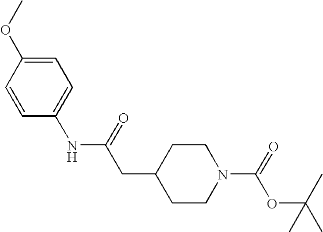 |
| 5 | 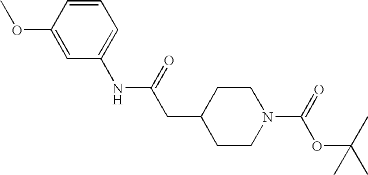 |
| 6 | 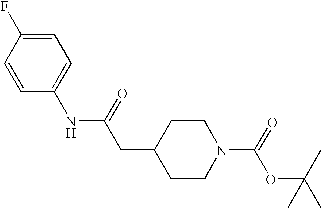 |
| 7 | 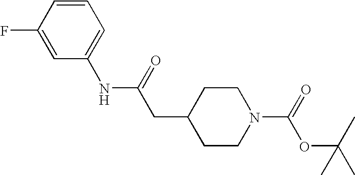 |
| 8 | 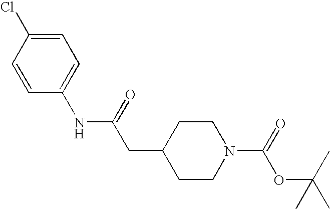 |
| 9 | 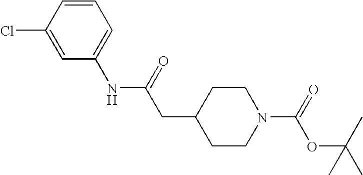 |
| 10 | 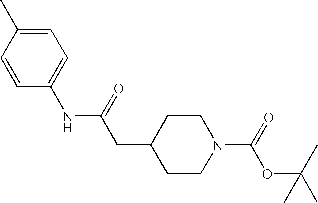 |
| 11 | 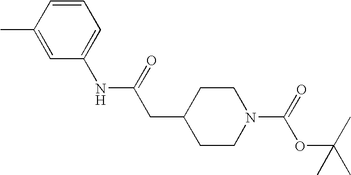 |
| 12 | 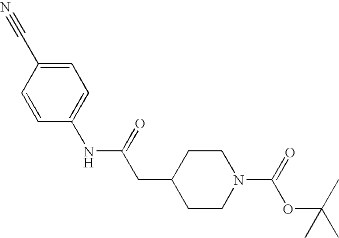 |
| 13 | 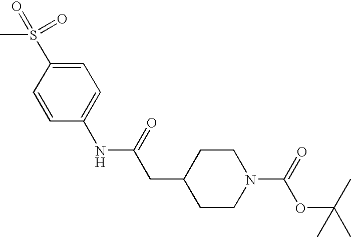 |
| 14 | 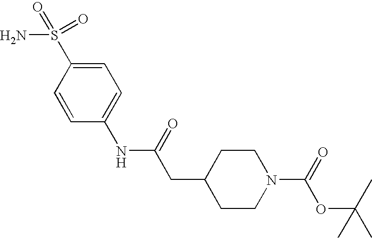 |
| 15 | 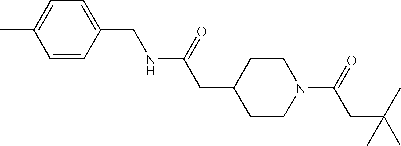 |
| 16 | 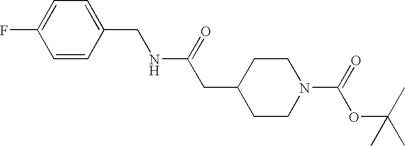 |
| 17 | 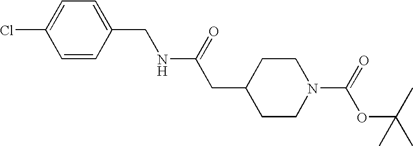 |
| 18 | 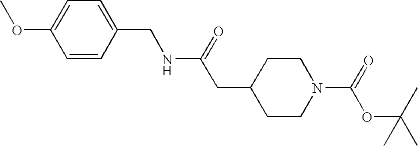 |
| 19 | 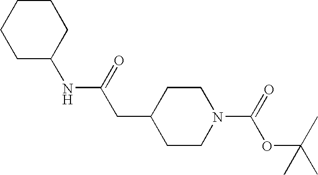 |
| 20 | 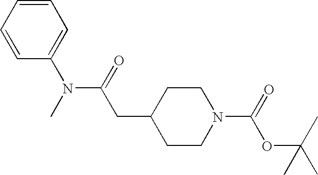 |
| 21 | 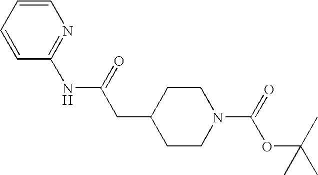 |
| 22 | 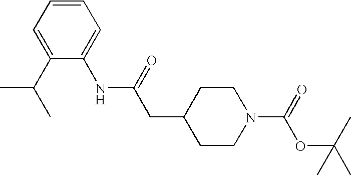 |
| 23 | 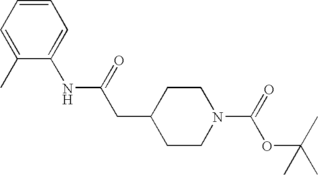 |
| 24 | 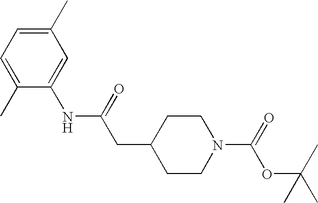 |
| 25 | 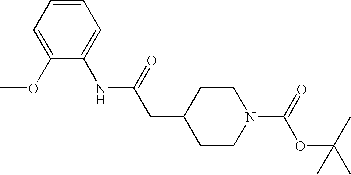 |
| 26 | 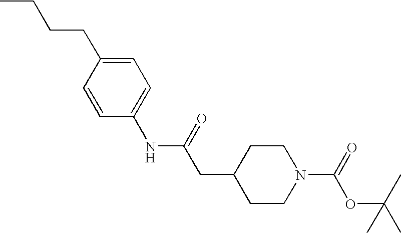 |
| 27 | 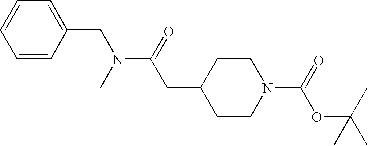 |
| 28 | 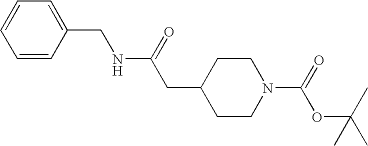 |

### General Method B: Boc Deprotection of Intermediate 3-28

To a solution of the previous N—BOC intermediates 3-28 (synthesis described in general method A) in DCM (1 ml) was added Trifluoroacetic acid (700 μL). The reaction mixtures were stirred 2 hours at RT. The resulting mixtures were evaporated with the GENEVAC, dissolved in EtOAc and washed several time with an aqueous solution of NaOH (2M). The organic layers were dried (MgSO₄) and concentrated under reduced pressure to afford after dry evaporation the desired intermediates as free base.

### General Method C: Synthesis of Compounds I-19, 23-28, 50

To a solution of the previous piperidine intermediates (synthesis described in general method B) (0.05 mmol), in ACN (2 ml) was added (2-Chloro-ethyl)-diphenyl-amine (J. Med. Chem. 1992, 35, 1042-1049) (16 mg; 0.07 mmol), powdered sodium iodide (7.5 mg; 0.05 mmol) and DIEA (20 μL; 0.12 mmol). The mixture was heated at 150° C. under microwave irradiation for 1 hour. The resulting mixtures were evaporated under vacuum, dissolved in DCM and washed several time with a saturated aqueous solution of NaHCO₃. The organic layers were dried (MgSO₄) and concentrated under reduced pressure. The crude was purified with a flash chromatography on silica gel (eluent: DCM/MeOH), then transferred to an ISOLUTE® SCX2 column and washed with MeOH and with a solution of 5%.aqueous ammonia in MeOH to afford after dry evaporation the desired compounds (table 4).

TABLE 4

| Compound | Structure |
|---|---|
| 1 | 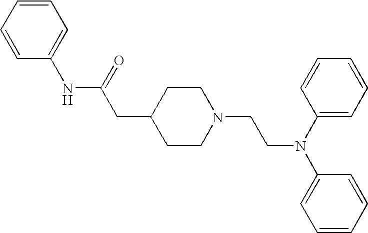 |
| 2 | 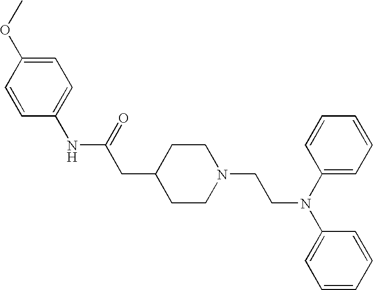 |
| 3 | 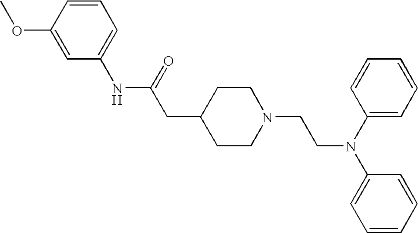 |
| 4 | 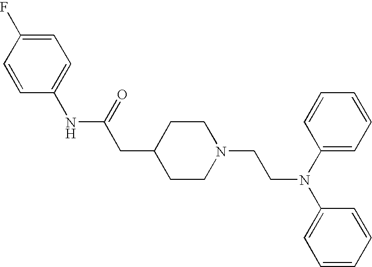 |
| 5 | 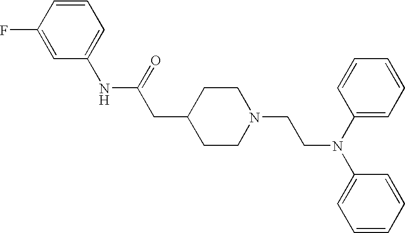 |
| 6 | 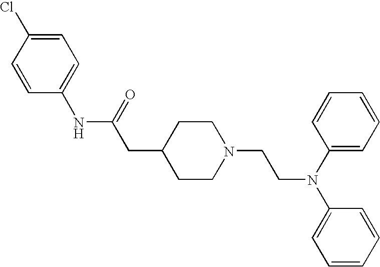 |
| 7 | 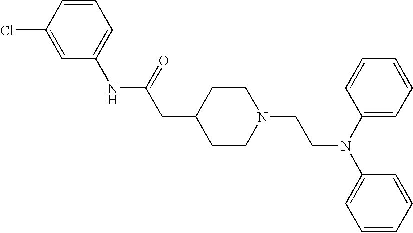 |
| 8 | 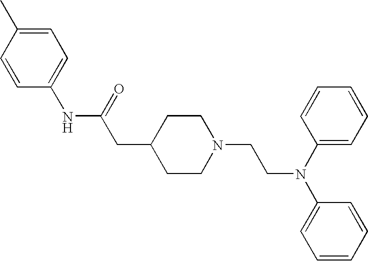 |
| 9 | 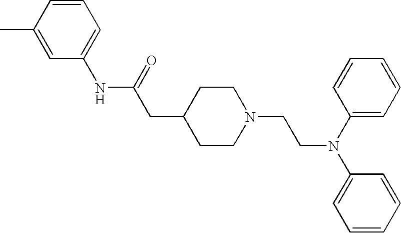 |
| 10 | 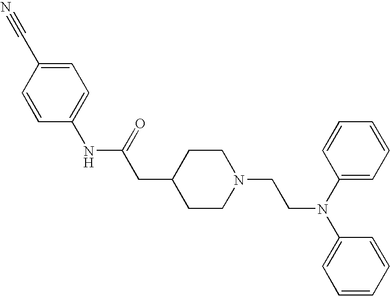 |
| 11 | 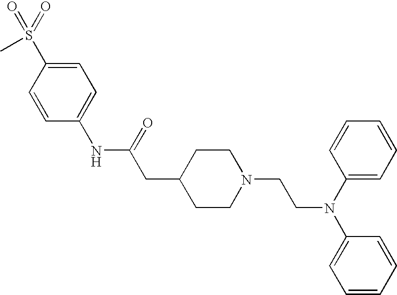 |
| 12 | 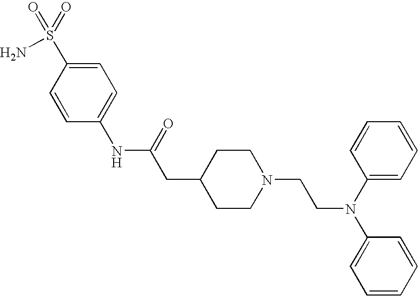 |
| 13 | 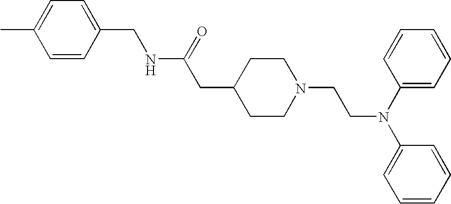 |
| 14 | 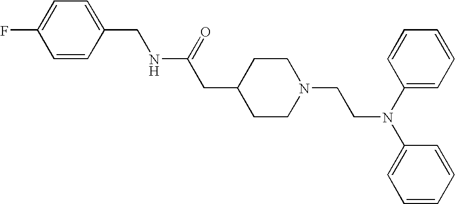 |
| 15 | 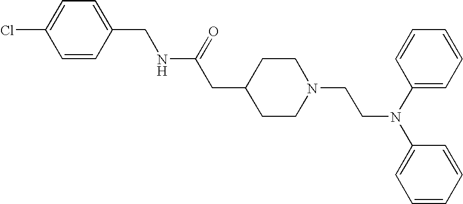 |
| 16 | 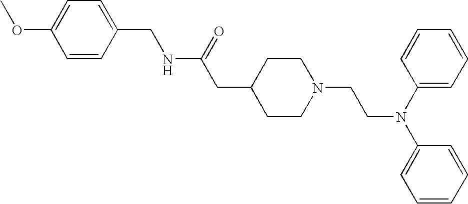 |
| 17 | 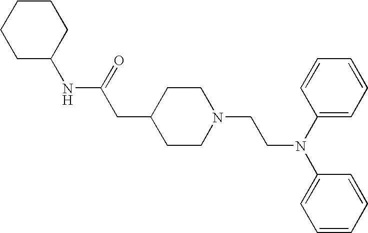 |
| 18 | 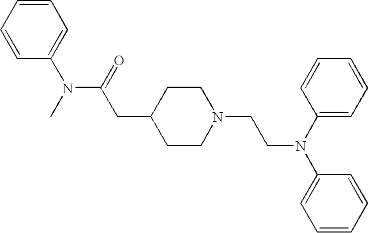 |
| 19 | 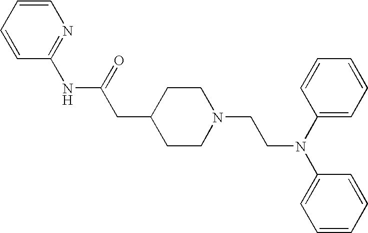 |
| 23 | 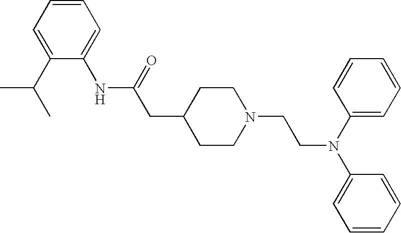 |
| 24 | 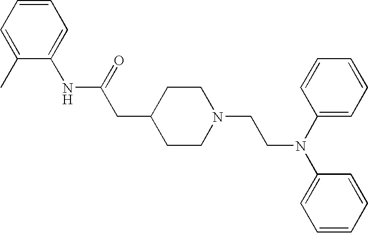 |
| 25 | 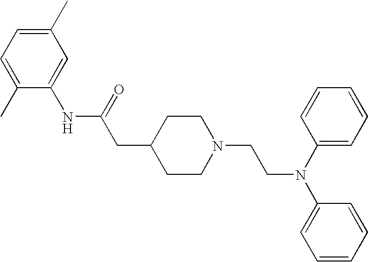 |
| 26 | 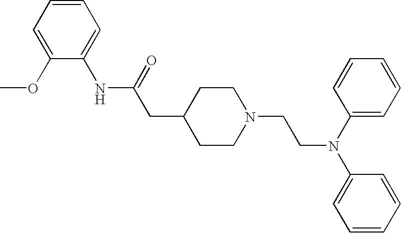 |
| 27 | 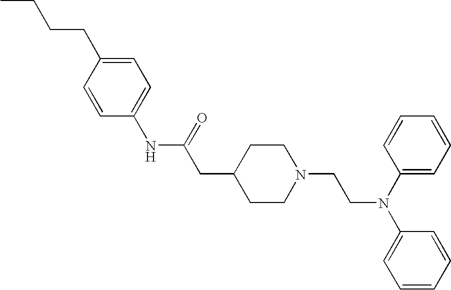 |
| 28 | 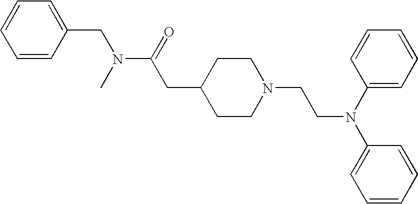 |
| 50 | 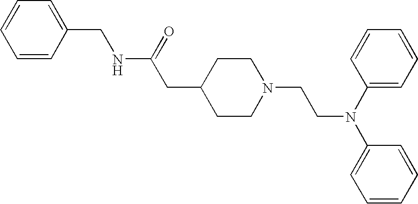 |

### Synthesis of Intermediate 29: 4-[(4-methanesulfonyl-phenylcarbamoyl)-methyl]-piperidine-1-carboxylic acid tert-butyl ester

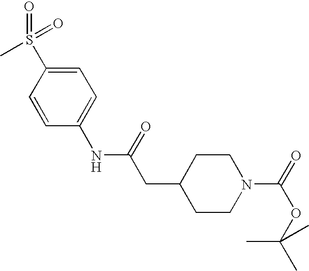

A solution of HOBt (282 mg; 0.88 mmol) and TBTU (135 mg; 0.88 mmol) in DMF (1 ml) was added to a solution of 1-BOC-piperidine-4-ylacetic acid (200 mg; 0.8 mmol) and the corresponding 4-(methylsulfonyl)aniline (137 mg, 0.8 mmol) in DMF (1 ml) followed by TEA (300 μL; 2.2 mmol). The reaction mixture was stirred 1 hour at RT and 2 hours at 50° C. The resulting mixture was evaporated under vacuum, dissolved in DCM and washed with an aqueous solution of NaHCO₃ and water. The organic layers were transferred to an ISOLUTE® PE-AX column and then to an ISOLUTE® SCX2 column using MeOH as eluent, to afford after dry evaporation under reduced pressure the desired intermediate.

### Synthesis of Intermediate 30: N-(4-methanesulfonyl-phenyl)-2-piperidin-4-yl-acetamide

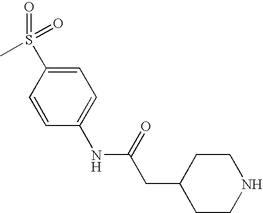

To a solution of 4-[(4-methanesulfonyl-phenylcarbamoyl)-methyl]-piperidine-1-carboxylic acid tert-butyl ester in DCM (1 ml) was added TFA (700 μL). The reaction mixture was stirred 2 hours at RT. The resulting mixture was evaporated under vacuum, dissolved in EtOAc and washed several time with an aqueous solution of NaOH (2M). The organic layer was dried (MgSO₄) and concentrated under reduced pressure to afford after dry evaporation the title intermediate as free base.

### Synthesis of Intermediate 31: 2-[1-(2-hydroxy-ethyl)-piperidin-4-yl]-N-(4-methanesulfonyl-phenyl)-acetamide

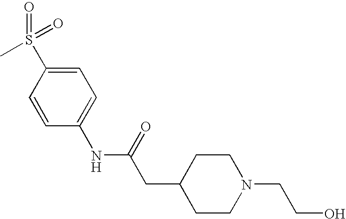

To a solution of N-(4-methanesulfonyl-phenyl)-2-piperidin-4-yl-acetamide (5.9 g, 20 mmol), in ACN (80 ml) was added 2-Chloro-ethanol (1.4 mL, 21 mmol), powdered sodium iodide (3 g; 20 mmol) and DIEA (6.6 mL; 40 nmol). The mixture was stirred at RT overnight then heated at 100° C. for 2 days. The mixture was evaporated under vacuum, dissolved in DCM and washed several time with a saturated aqueous solution of NaHCO₃. The organic layers were dried (MgSO₄) and concentrated under reduced pressure. The crude was purified with a flash chromatography on silica gel (eluent: DCM/MeOH) to afford after dry evaporation the title intermediate.

### Synthesis of Intermediate 32: 2-[1-(2-chloro-ethyl)-piperidin-4-yl]-N-(4-methanesulfonyl-phenyl)acetamide hydrochloride

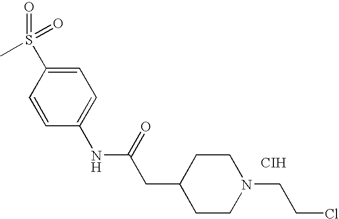

To a solution of 2-[1-(2-hydroxy-ethyl)-piperidin-4-yl]-N-(4-methanesulfonyl-phenyl)-acetamide (710 mg; 2.09 mmol) in anhydrous toluene (1 ml) was added while stirring a solution of thionyl chloride (305 μL, 4.18 mmol) in anhydrous toluene (1 mL) so that the temperature remained between 25 and 30° C. The reaction was stirred at RT for 3 days and then concentrated under reduced pressure. The hydrochloride salt was scratched in Et₂O, filtered and washed with Et₂O to afford the title intermediate.

### Synthesis of Intermediate 33: N-(4-methanesulfonyl-phenyl)-2-[1-(2-phenylamino-ethyl)-piperidin-4-yl]-acetamide

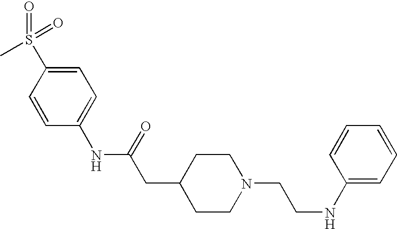

A mixture of 2-[1-(2-chloro-ethyl)-piperidin-4-yl]-N-(4-methanesulfonyl-phenyl)acetamide hydrochloride (232 mg, 0.59 mmol), aniline (109 mg, 1.17 mmol), powdered sodium iodide (88.4 mg, 0.59 mmol) and DIEA (204 μL, 1.18 mmol) in ACN (1 ml) were heated at 100° C. for 10 minutes in a microwave. The crude reaction mixture was filtered through a cotton wool plug and concentrated under vacuum. The residue was purified by silica gel chromatography (eluent: EtOAc/Cyclohexane) to afford after dry evaporation under vacuum the title intermediate.

### Synthesis of compounds: 30, 31, 47, 48

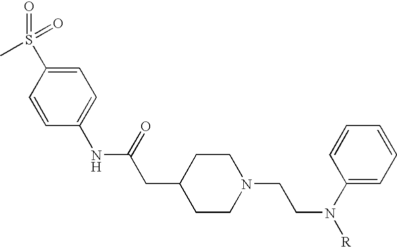

To a mixture of N-(4-methanesulfonyl-phenyl)-2-[1-(2-phenylamino-ethyl)-piperidin-4-yl]-acetamide (45.6 mg, 0.11 mmol), TEA (30.5 μL, 0.22 mmol) in ACN (1 ml) was added dropwise at 0° C. the corresponding alkyl halide (0.11 mmol). The mixture was then stirred overnight at 70° C. The crude reaction mixture was concentrated under reduced pressure, DCM was then added and the organic layer was washed with water, dried over MgSO₄ and concentrated under reduced pressure. The residue was purified by silica gel chromatography (eluent: DCM/MeOH) to afford after dry evaporation under vacuum the title compounds (Table 5).

TABLE 5

| compound | Structure |
|---|---|
| 30 | 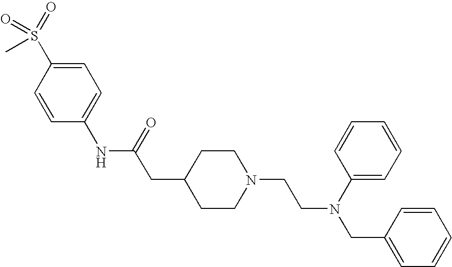 |
| 31 | 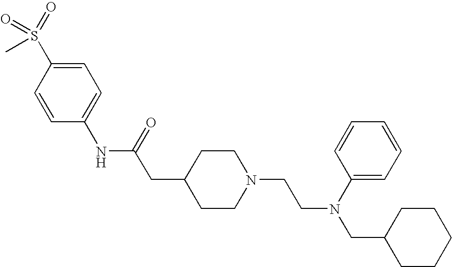 |
| 47 | 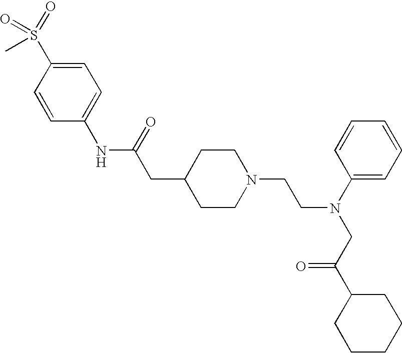 |
| 48 | 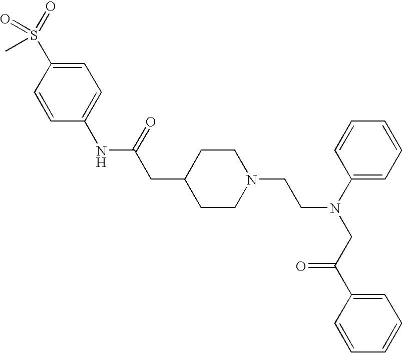 |

### Synthesis of Compounds: 36-37

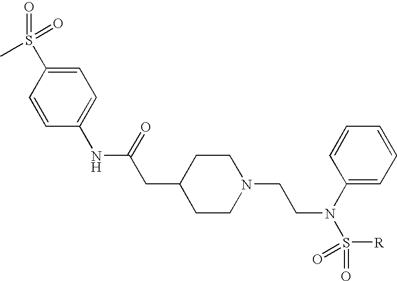

To a mixture of N-(4-methanesulfonyl-phenyl)-2-[1-(2-phenylamino-ethyl)-piperidin-4-yl]-acetamide (45.6 mg, 0.11 mmol), TEA (30.5 μL, 0.22 mmol) in ACN (1 ml) was added dropwise at 0° C. the corresponding sulfonyl halide (0.11 mmol). The mixture was then stirred overnight at 70° C. The crude reaction mixture was concentrated under reduced pressure, DCM was then added and the organic-layer was washed with a saturated aqueous solution of NaHCO₃, dried over MgSO₄ and concentrated under reduced pressure. The residue was purified by silica gel chromatography (eluent: DCM/MeOH) to afford after dry evaporation under vacuum the title compounds (table 6).

TABLE 6

| compound | Structure |
|---|---|
| 36 | 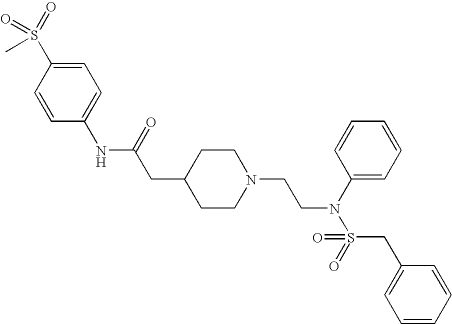 |
| 37 | 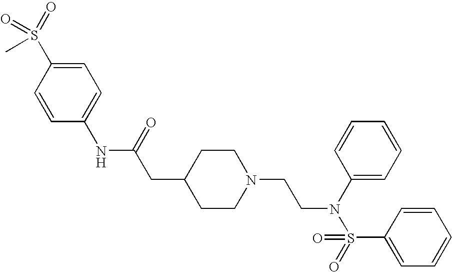 |

### Synthesis of Compounds: 33-35, 45-46

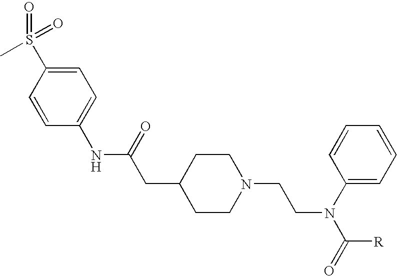

To a mixture of N-(4-methanesulfonyl-phenyl)-2-[1-(2-phenylamino-ethyl)-piperidin-4-yl]-acetamide (45.6 mg, 0.11 mmol), TEA (30.5 μL, 0.22 mmol) in ACN (1 ml) was added dropwise at 0° C. the corresponding acid chloride (0.11 mmol). The mixture was then stirred overnight at 70° C. The crude reaction mixture was concentrated under reduced pressure, DCM was then added and the organic layer was washed with a saturated aqueous solution of NaHCO₃, dried over MgSO₄ and concentrated under reduced pressure. The residue was purified by silica gel chromatography (eluent: DCM/MeOH) to afford after dry evaporation under vacuum the title compounds (table 7).

TABLE 7

| compound | Structure |
|---|---|
| 33 | 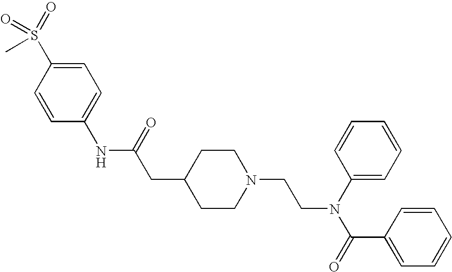 |
| 34 | 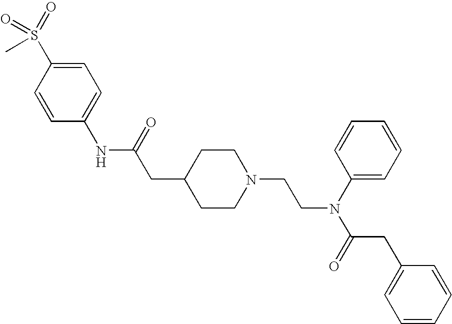 |
| 35 | 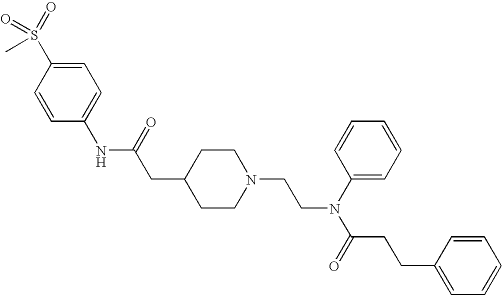 |
| 45 | 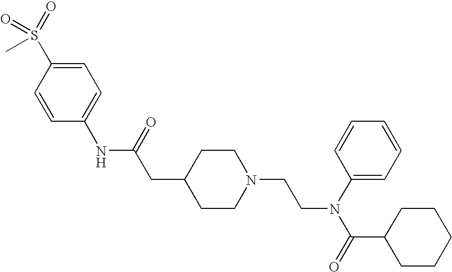 |
| 46 | 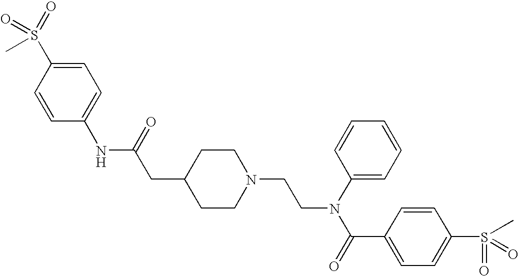 |

### Synthesis of Compounds: 32, 38-39

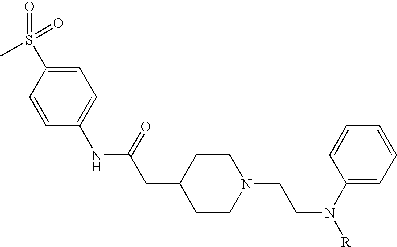

To a solution of 2-[1-(2-chloro-ethyl)-piperidin-4-yl]-N-(4-methanesulfonyl-phenyl)acetamide hydrochloride (43.3 mg, 0.11 mmol) in ACN/DMF (1:1) were added powdered sodium iodide (16.5 mg, 0.11 mmol), N-phenylalkylamine (0.11 mmol) and DIEA (36 μL, 0.22 mmol). The reaction mixture was heated at 160° C. for 1 hour under microwave irradiation and concentrated under reduced pressure. The crude was dissolved in DCM and water. The aqueous layer was extracted with DCM, and the organic layer was then washed with brine. The residue was purified by silica gel chromatography (eluent: DCM/MeOH) to afford after dry evaporation under vacuum the title compounds (table 8).

TABLE 8

| compound | Structure |
|---|---|
| 32 | 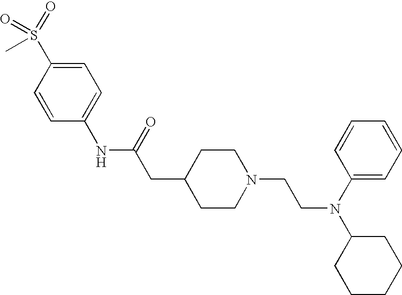 |
| 38 | 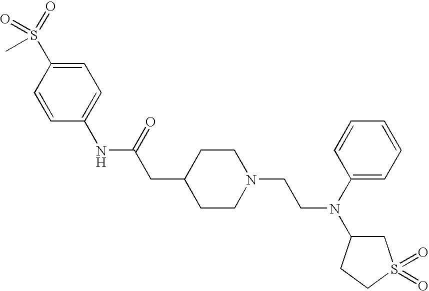 |
| 39 | 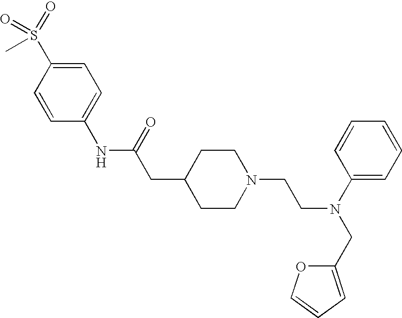 |

### Synthesis of Compound 41: 4-[(2-(4-[(4-methanesulfonyl-phenylcarbamoyl)-methyl]-piperidin-1-yl)-ethyl)-phenyl-amino]-piperidine-1-carboxylic acid tert-butyl ester

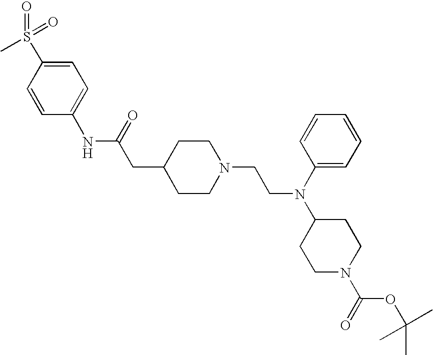

To a mixture of N-(4-methanesulfonyl-phenyl)-2-[1-(2-phenylamino-ethyl)-piperidin-4-yl]-acetamide (45.6 mg, 0.11 mmol), TEA (30.5 μL, 0.22 mmol) in ACN (1 ml) was added dropwise at 0° C. the N—BOC-4-chloropiperidine (24.1 mg, 0.11 mmol). The mixture was then stirred overnight at 70° C. The crude reaction mixture was concentrated under reduced pressure, DCM was then added and the organic layer was washed with water, dried over MgSO₄ and concentrated under reduced pressure. The residue was purified by silica gel chromatography (eluent: DCM/MeOH) to afford after dry evaporation under vacuum the title compound.

### Synthesis of compound 42: N-(4-methanesulfonyl-phenyl)-2-{1-[2-(phenyl-piperidin-4-yl-amino)-ethyl]-piperidine-4-yl}-acetamide

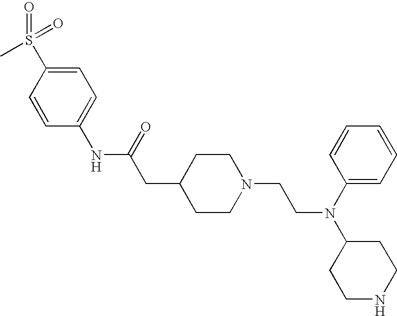

To a solution of 4-[(2-{4-[(4-methanesulfonyl-phenylcarbamoyl)-methyl]-piperidin-1-yl}-ethyl)-phenyl-amino]-piperidine-1-carboxylic acid tert-butyl ester in DCM (1 ml) was added TFA (700 μL). The reaction mixture was stirred 2 hours at RT. The resulting mixture was evaporated under vacuum, dissolved in EtOAc and washed several time with an aqueous solution of NaOH (2M). The organic layer was dried (MgSO₄) and concentrated under reduced pressure to afford after dry evaporation the desired compound as a free base.

### Synthesis of Compound 43: N-(4-methanesulfonyl-phenyl)-2-[1-[2-{phenyl-[1-(2,2,2-trifluoro-acetyl)-piperidin-4-yl]-amino}-ethyl)-piperidin-4-yl]-acetamide

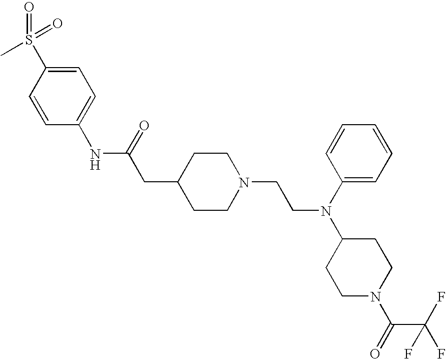

To a mixture of N-(4-methanesulfonyl-phenyl)-2-{1-[2-(phenyl-piperidin-4-yl-amino)-ethyl]-piperidine-4-yl}-acetamide (54.8 mg, 0.11 mmol), TEA (30.5 μL, 0.22 mmol) in ACN (1 ml) was added dropwise at 0° C. the trifluoroacetyl chloride (14.6 mg, 0.11 mmol). The mixture was then stirred overnight at 70° C. The crude reaction mixture was concentrated under reduced pressure, DCM was then added and the organic layer was washed with a saturated aqueous solution of NaHCO₃, dried over MgSO₄ and concentrated under reduced pressure. The residue was purified by silica gel chromatography (eluent: DCM/MeOH) to afford after dry evaporation under vacuum the title compound.

### Synthesis of compound 44: N-(4-methanesulfonyl-phenyl)-2-(1-(2-[(1-methanesulfonyl-piperidin-4-yl)-phenyl-amino]-ethyl)-piperidin-4-yl)-acetamide

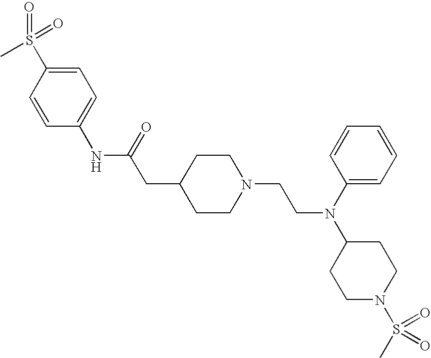

To a mixture of N-(4-methanesulfonyl-phenyl)-2-{1-[2-(phenyl-piperidin-4-yl-amino)-ethyl]-piperidine-4-yl}-acetamide (54.8 mg, 0.11 mmol), TEA (30.5 μL, 0.22 mmol) in ACN (1 ml) was added dropwise at 0° C. the methanesulfonyl chloride (8.5 mL, 0.11 mmol). The mixture was then stirred overnight at 70° C. The crude reaction mixture was concentrated under reduced pressure, DCM was then added and the organic layer was washed with a saturated aqueous solution of NaHCO₃, dried over MgSO₄ and concentrated under reduced pressure. The residue was purified by silica gel chromatography (eluent: DCM/MeOH) to afford after dry evaporation under vacuum the title compound.

### Synthesis of Compounds: 20, 22

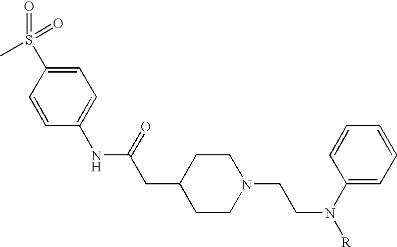

A suspension of boronic acid (0.2 mmol), Cu(OAc)₂.H₂O (2 mg, 0.01 mmol), and powdered 4 Å molecular sieves (75 mg) in DCM (1 ml) was stirred for 5 minutes at RT. To this stirring suspension was added N-(4-methanesulfonyl-phenyl)-2-[1-(2-phenylamino-ethyl)-piperidin-4-yl]-acetamide (41.5 mg, 0.1 mmol). The reaction mixture was then sealed with a rubber septa, and stirred under an atmosphere of O₂. The reaction was stirred with a magnetic stir bar for a period of 24H. The crude was filtered through a plug of celite to remove the molecular sieves and any insoluble products and the organic layer was concentrated under vacuum to afford the crude mixture. The product was isolated on SPE-SCX (Table 9).

TABLE 9

| compound | Structure |
|---|---|
| 20 | 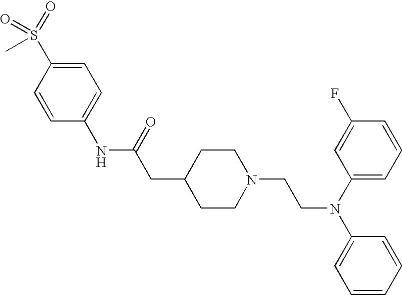 |
| 22 | 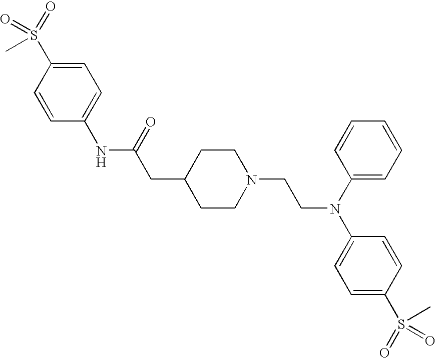 |

### Synthesis of Intermediate 36: 2-(1-[2(3-fluoro-phenylamino)-ethyl]-piperidin-4-yl)-N-(4-methanesulfonyl-phenyl)-acetamide

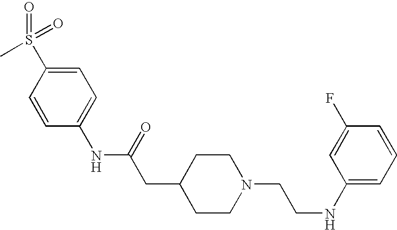

A mixture of 2-[1-(2-chloro-ethyl)-piperidin-4-yl]-N-(4-methanesulfonyl-phenyl)acetamide hydrochloride (232 mg, 0.59 mmol), 3-fluoroaniline (130 mg, 1.17 mmol), powdered sodium iodide (88.4 mg, 0.59 mmol) and DIEA (204 μL, 1.18 mmol) in ACN (1 ml) was heated at 100° C. for 10 minutes in a microwave. The crude reaction mixture was filtered through a cotton wool plug and concentrated under vacuum. The residue was purified by silica gel chromatography (eluent: EtOAc/Cyclohexane) to afford after dry evaporation under vacuum the title intermediate.

### Synthesis of compound 21: 2-(1-{2-[(3-fluoro-phenyl)-(4-methanesulfonyl-phenyl)-amino]-ethyl}-piperidin-4-yl)-N-(4-methanesulfonyl-phenyl)-acetamide

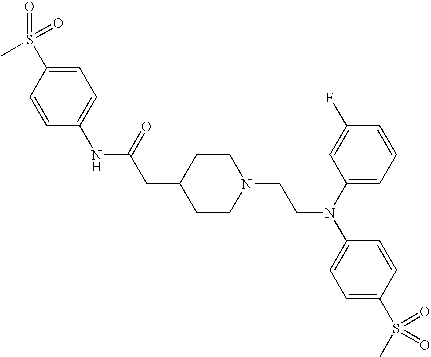

A suspension of 4-(methylsulfonyl)phenylboronic acid (40 mg, 0.2 mmol), Cu(OAc)₂.H₂O (2 mg, 0.01 mmol), and powdered 4 Å molecular sieves (75 mg) in DCM (1 ml) was stirred for 5 minutes at RT. To this stirring suspension was added 2-{1-[2(3-fluoro-phenylamino)-ethyl]-piperidin-4-yl}-N-(4-methanesulfonyl-phenyl)-acetamide (43.3 mg, 0.1 mmol). The reaction mixture was then sealed with a rubber septa, and stirred under an atmosphere of O₂. The reaction was stirred with a magnetic stir bar for a period of 24H. The crude was filtered through a plug of celite to remove the molecular sieves and any insoluble products and the organic layer was concentrated under vacuum to afford the crude mixture. The product was isolated on SPE-SCX.

### Synthesis of intermediate 37: 2-[1-(2-hydroxy-1-methyl-ethyl)-piperidin-4-yl]-N-(4-methanesulfonyl-phenyl)-acetamide

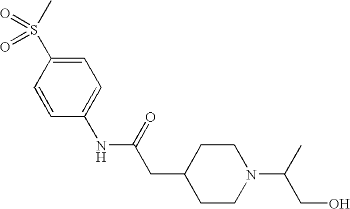

To a solution of N-(4-methanesulfonyl-phenyl)-2-piperidin-4-yl-acetamide (852 mg, 2.88 mmol) in ACN (19 mL) were added 2-chloro-propan-1-ol (271 mg; 2.88 mmol), powdered sodium iodide (432 mg; 2.88 mmol) and DIEA (952 μL; 5.76 mmol). The mixture was heated at 150° C. for 25 minutes then filtered over silica gel and concentrated under reduced pressure to afford the title intermediate.

### Synthesis of intermediate 38: 2-[1-(2-chloro-1-methyl-ethyl)-piperidin-4-yl]-N-(4-methanesulfonyl-phenyl)acetamide hydrochloride

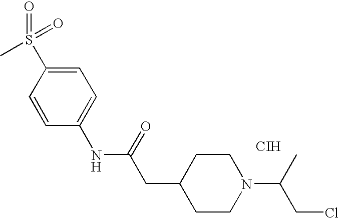

To a solution of 2-[(1-(2-hydroxy-1-methyl-ethyl)-piperidin-4-yl]-N-(4-methanesulfonyl-phenyl)-acetamide (740 mg; 2.09 mmol) in anhydrous toluene (1 ml) was added while stirring a solution of thionyl chloride (305 μL, 4.18 mmol) in anhydrous toluene (1 mL) so that the temperature remained between 25 and 30° C. The reaction was stirred overnight at RT and then concentrated under reduced pressure. The hydrochloride salt was scratched in Et₂O, filtered and washed with Et₂O to afford the title intermediate.

### Synthesis of Compound 29: 2-[1-(2-diphenylamino-1-methyl-ethyl)-piperidin-4-yl]-N-(4-methanesulfonyl-phenyl)acetamide

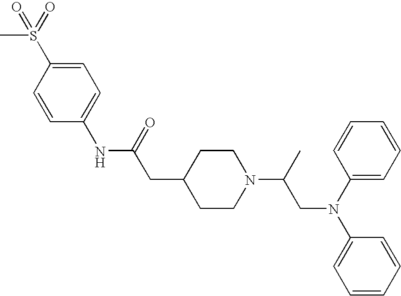

To a solution of 2-[1-(2-chloro-1-methyl-ethyl)-piperidin-4-yl]-N-(4-methanesulfonyl-phenyl)acetamide hydrochloride. (44.9 mg, 0.11 mmol) in ACN/DMF (1:1) were added powdered sodium iodide (16.5 mg, 0.11 mmol), diphenylamine (18.6 mg, 0.11 mmol) and DIEA (36 μL, 0.22 mmol). The reaction mixture was heated at 160° C. for 1 hour under microwave irradiation and concentrated under reduced pressure. The crude was dissolved in DCM and water. The aqueous layer was extracted with DCM, and the organic layer was then washed with brine. The residue was purified by silica gel chromatography (eluent: DCM/MeOH) to afford after dry evaporation under vacuum the title compound.

### Synthesis of Compound 49: 1-[1-(2-diphenylamino-ethyl)-piperidin-4-yl]-3-phenyl-propan-2-one

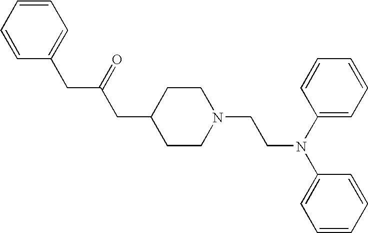

A stirred solution of [1-(2-Diphenylamino-ethyl)-piperidin-4-yl]-acetic acid (0.411 g, 1 mmol) in acetonitrile (10 mL) was treated with TBTU (0.385 g, 1.2 mmol), DIEA (1.04 mL, 6 mmol) and N,O-dimethylhydroxylamine hydrochloride (0.117 g, 1.2 mmol), then strirred at room temperature overnight. The volatiles were removed under vacuum. The residue was partitioned between AcOEt and saturated aqueous NaHCO₃. The aqueous layer was re-extracted twice with AcOEt. Combined organics were washed with brine, dried over MgSO₄ and concentrated to provide the 2-[1-(2-diphenylamino-ethyl)-piperidin-4-yl]-N-methoxy-N-methyl-acetamide intermediates which was used in subsequent reactions.

A solution of n-BuLi (1M in hexanes, 1 mL, 1 mmol) was added rapidly to a stirred solution of DABCO (111 mg, 1 mmol) in anhydrous toluene (5 mL). The color of the mixture became yellow and after heating at 80° C. for 30 min., bright yellow needles were formed. Hereafter, the mixture was cooled to room temperature and treated with a solution of 2-[1-(2-diphenylamino-ethyl)-piperidin-4-yl]-N-methoxy-N-methyl-acetamide (316 mg, 0.83 mmol) in anhydrous toluene (3 mL). The needles soon disappeared and the reaction mixture was then stirred for another 60 min., washed with saturated NaHCO₃ and brine, dried over MgSO₄ and concentrated in vacuo. The crude was purified by column chromatography using a gradient of DCM/MeOH to obtain the title compound.

### Synthesis of Compounds 51-68, 72 and 75-98 (see Table 10)

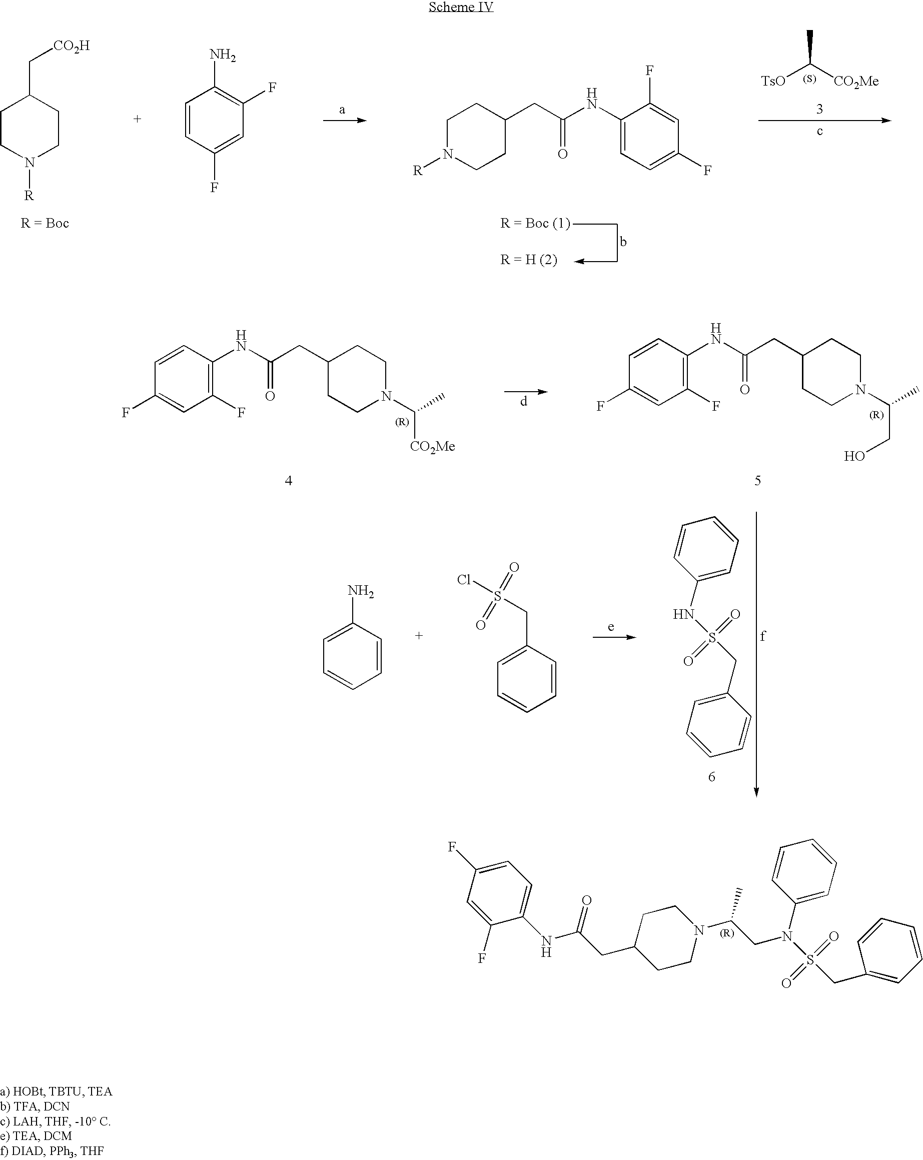

### Synthesis of Intermediate 39: tert-butyl 4-(2-(2,4-difluorophenylamino)-2-oxoethyl)piperidine-1-carboxylate

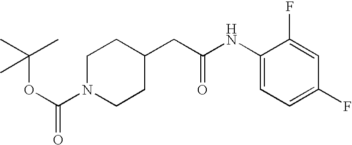

To a solution of HOBt (26.4 mmol; 1.2 eq) and TBTU (26.4 mmol; 1.2 eq) was added the 2,4-difluoroaniline (26 mmol; 1.2 eq) in DMF (200 mL) followed by the addition of TEA (26.4 mmol; 1.2 eq) and 2-(1-(tert-butoxycarbonyl)piperidin-4-yl)acetic acid (22 mmol; 1 eq). The reaction mixture was stirred at RT overnight. After concentrating the reaction mixture under reduced pressure, the residue was taken up in DCM and washed three times with saturated NaHCO₃ solution. The organic phase was separated, dried (over MgSO₄) and after filtration, the volatiles were removed under reduced pressure. The solid thus obtained was triturated with diethyl ether, re-filtered and dried in vacuo to obtain the title intermediate.

### Synthesis of Intermediate 40: N-(2,4-difluorophenyl)-2-(piperidin-4-yl)acetamide

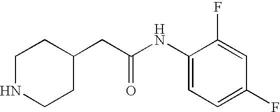

To a DCM (150 mL) solution of tert-butyl 4-(2-(2,4-difluorophenylamino)-2-oxoethyl)piperidine-1-carboxylate was added TFA (200 mmol; 10 eq) and the reaction mixture was stirred at RT for 1 h. After concentrating this reaction mixture under reduced pressure, the obtained residue was taken up in pH 14 aqueous solution (NaOH). The aqueous phase was extracted three times with EtOAc, and the combined organic phase was dried over MgSO₄, filtered and the volatiles were removed in vacuo. The solid thus obtained was triturated with diethyl ether, re-filtered and dried under vacuum.

### Synthesis of Intermediate 41: (S)-methyl 2-(tosyloxy)propanoate

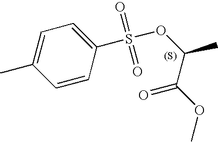

To an anhydrous DCM solution of lactate (20 mmol; 1 eq) and 4-methylbenzene-1-sulfonyl chloride (24 mmol; 1.2 eq) was added TEA (30.9 mmol; 1.55 eq) at 0° C. under inert atmosphere. The reaction mixture was stirred at RT for 1 day. After addition of water, the reaction mixture was subjected to DCM extraction. The organic phase was washed with saturated NaHCO₃, dried (MgSO₄) and after filtration and removal of the volatiles the title intermediate was obtained as crude product, which was further purified by silica-gel flash column chromatography (elution: 95% cyclohexane/5% EtOAc to 90% cyclohexane/10% EtOAc).

### Synthesis of Intermediate 42: (R)-methyl 2-(4-(2-(4-fluorophenyl)acetamido)piperidin-1-yl)propanoate

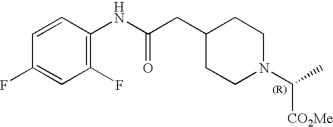

To the solution of N-(2,4-difluorophenyl)-2-(piperidin-4-yl)acetamide (1.84 mmol; 1 eq) was added DIEA (2.16 mmol; 1.2 eq) and (s)-methyl 2-(tosyloxy)propanoate (1.94 mmol 1.05 eq) in anhydrous MeCN (11 mL) under inert atmosphere. The reaction mixture was stirred under reflux for 1 day. The volatiles were then removed in vacuo and the obtained residue was taken up in DCM and washed three times with saturated NaHCO₃. The organic phase was separated, dried (MgSO₄) and concentrated under reduced pressure. The residue thus obtained was subjected to ISOLUTE SCX2™ column and washed with MeOH and with a solution of 5% aqueous ammonia in MeOH to obtain the title intermediate.

Synthesis of intermediate 43: (R)—N-(2,4-difluorophenyl)-2-(1-(1-hydroxypropan-2-yl)piperidin-4-yl)acetamide

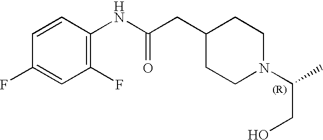

To the N-(2,4-difluorophenyl)-2-(piperidin-4-yl)acetamide was added 1 equiv of LiAlH₄ (0.93 mmol; 1 eq) in anhydrous THF (12 mL) at −10° C. under inert atmosphere. After stirring the reaction at −10° C. for 1 h, to the reaction milieu was added aqueous NaOH (8 M). The mixture thus obtained was then extracted, several times with EtOAc, the organic extracts were combined, dried (MgSO₄) and after filtration, the volatiles were removed in vacuo to obtain the title intermediate.

### Synthesis of Intermediate 45: N,1-diphenylmethane-sulfonamide

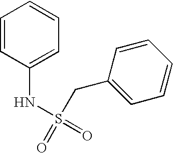

Triethylamine (3 mmol; 1.2 eq) was added to a solution containing aniline (3 mmol; 1.2 eq) and benzylsulfonyl chloride (2.5 mmol; 1 eq) in anhydrous DCM (5 mL) under inert atmosphere. The reaction mixture was then stirred at RT for 2 days whereupon HCl (1M) was added and the reaction mixture was extracted with DCM. The organic phases were combined and dried over MgSO₄. After filtering the MgSO₄ and removal of volatiles, a residue was obtained that was then subjected to silica-gel column chromatographic purification to afford this intermediate.

Note: All sulfonyl chlorides that were not commercially available were accessed through the procedure described in Nishiguchi et al, Synthesis, 2006, 4131 by using the commercially available halide, or via pseudohalides (mesylate, tosylate) from the alcohol precursor.

### General Method D: Example of (R)—N-(1-(1-(N,1-diphenylmethylsulfonamido)propan-2-yl)piperidin-4-yl)-2-(4-fluorophenyl)acetamide

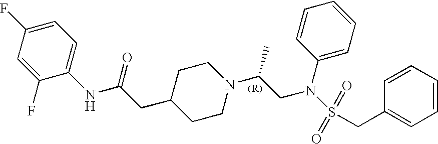

To the solution of (R)-2-(4-fluorophenyl)-N-(1-(1-hydroxypropan-2-yl)piperidin-4-yl)acetamide (0.169 mmol; 1 eq) was added PPh₃ (0.254 mmol; 1.5 eq), DIAD (0.254 mmol; 1.5 eq) and N,1-diphenylmethanesulfonamide (0.203 mmol; 1.2 eq) in anhydrous THF at 0° C. under inert atmosphere. After stirring the reaction mixture for 5 min at 0° C., it was allowed to warm up to ET whereupon it was further stirred for 2-3 hours. After concentrating the reaction mixture, the crude residue was subjected to ISOLUTE SCX2™ column and washed with MeOH and with a solution of 5% aqueous ammonia in MeOH. Thereafter the residue thus purified was further subjected to silica-gel column chromatography, with cyclohexane (100%) to cyclohexane/EtOAc (3:7) as typical eluant system to afford the title compounds (Table 10)

### Synthesis of Compounds 69-71 (see Table 10)

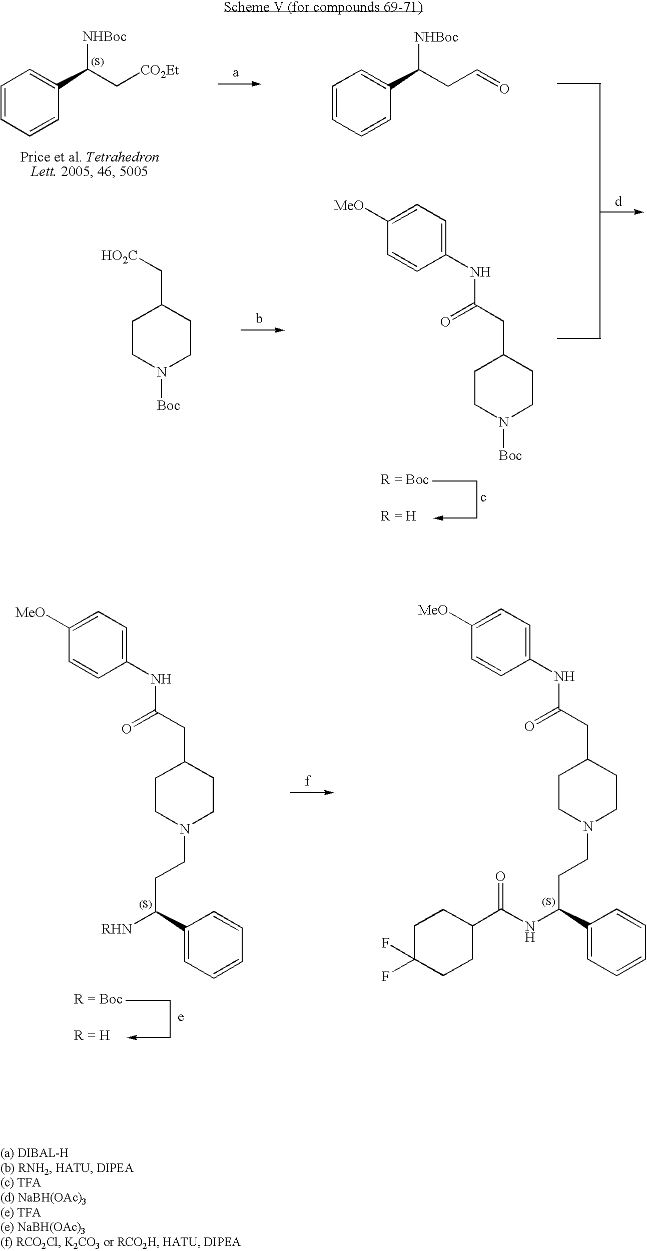

### Synthesis of Compounds 73-74 (see Table 10)

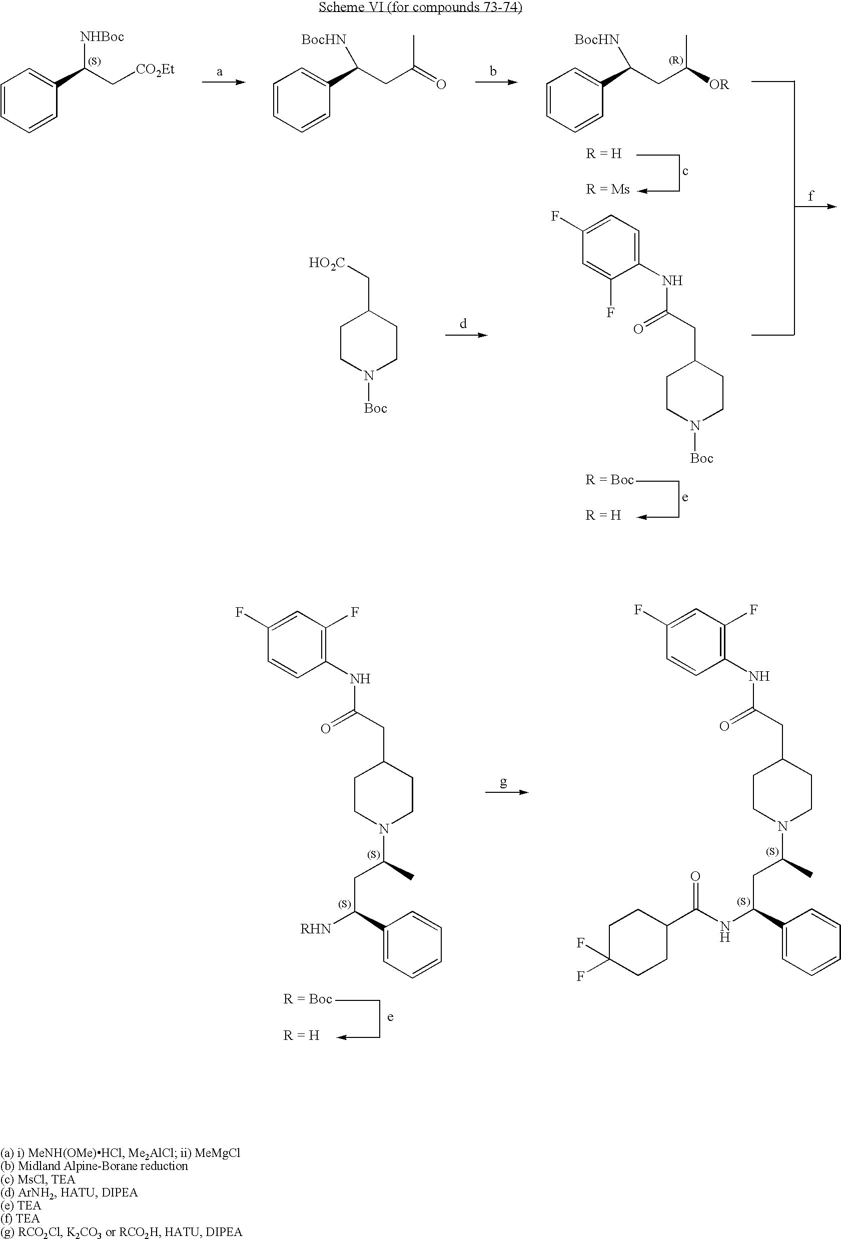

### Synthesis of Compounds 100-102 (see Table 10)

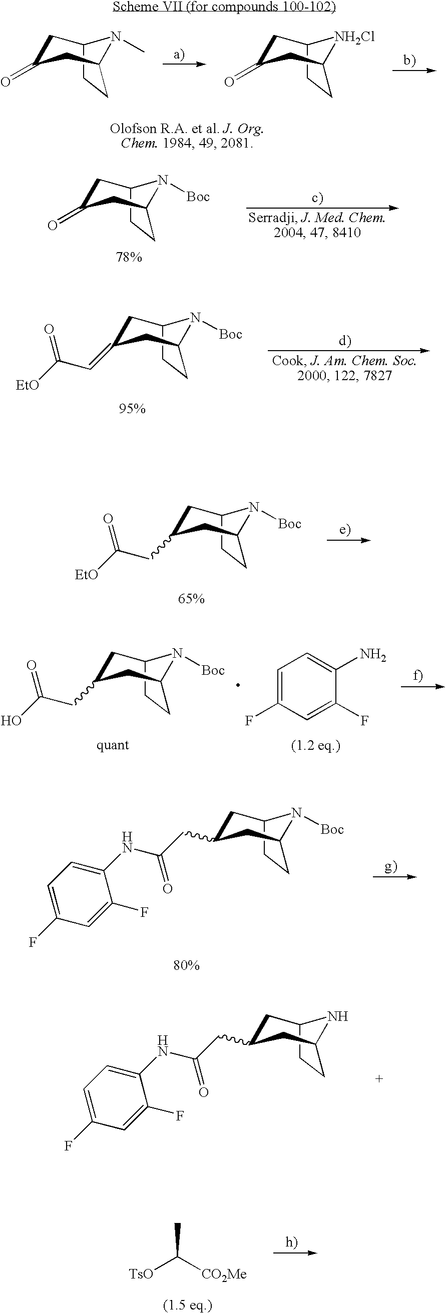

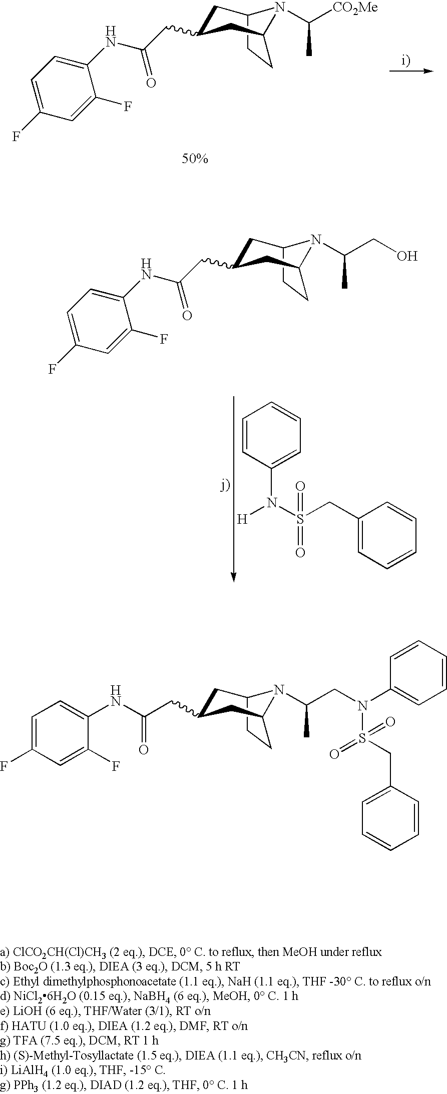

TABLE 10

| Compound | Structure |
|---|---|
| 51 | 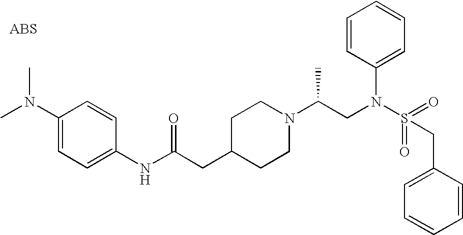 |
| 52 | 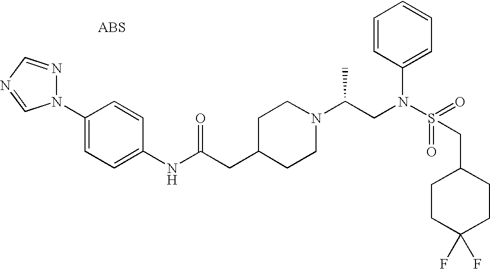 |
| 53 | 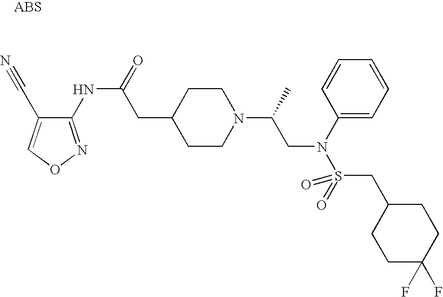 |
| 54 | 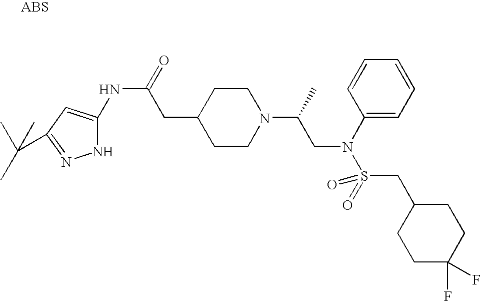 |
| 55 | 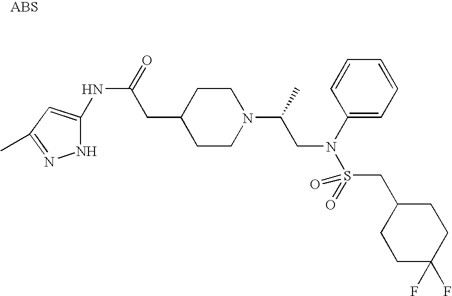 |
| 56 | 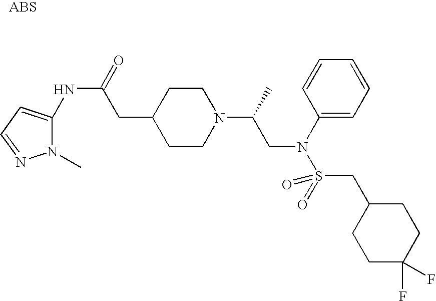 |
| 57 | 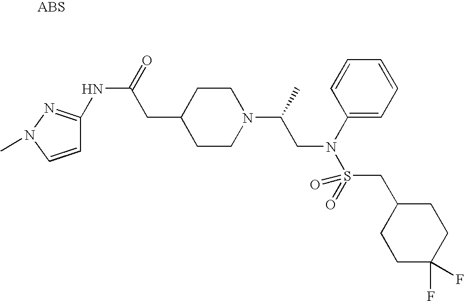 |
| 58 | 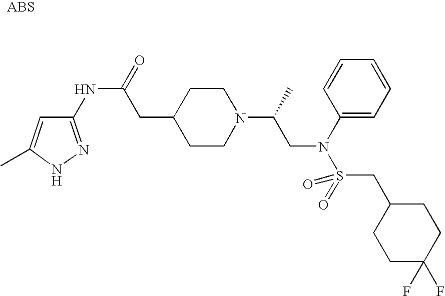 |
| 59 | 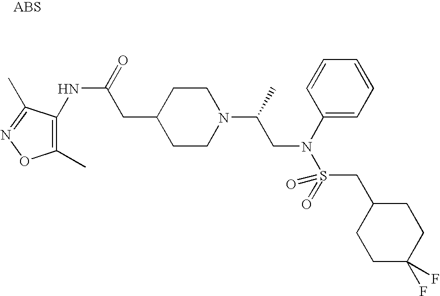 |
| 60 | 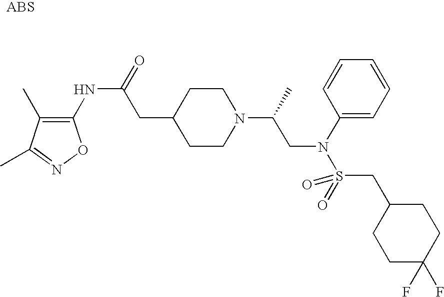 |
| 61 | 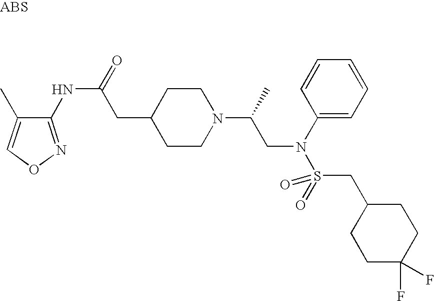 |
| 62 | 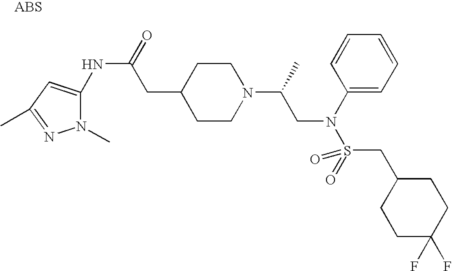 |
| 63 | 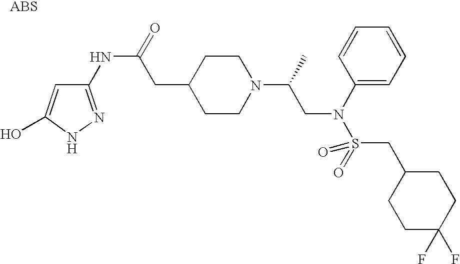 |
| 64 | 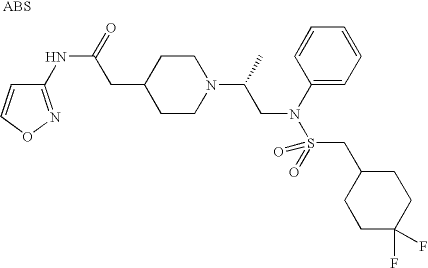 |
| 65 | 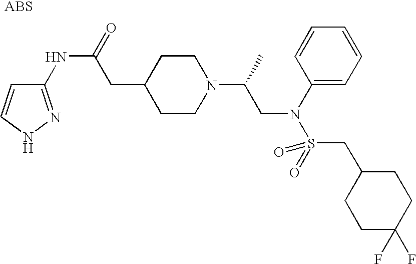 |
| 66 | 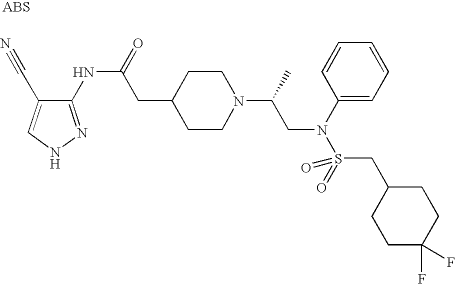 |
| 67 | 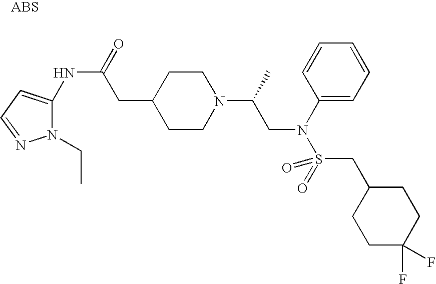 |
| 68 | 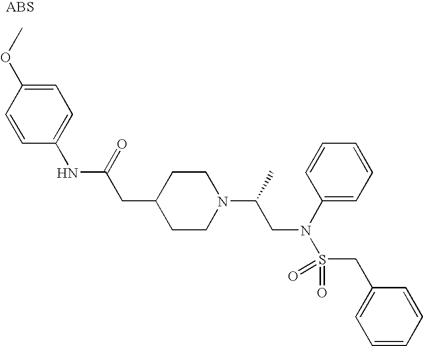 |
| 69 | 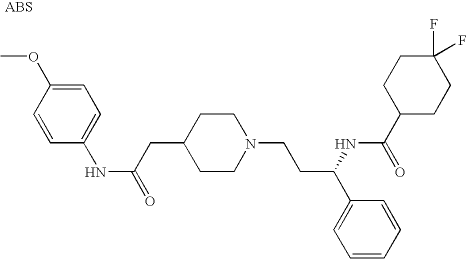 |
| 70 | 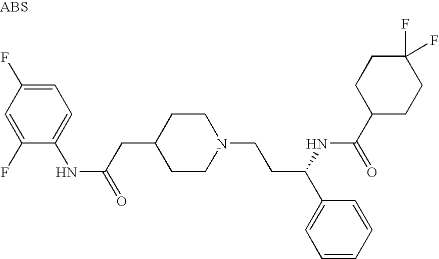 |
| 71 | 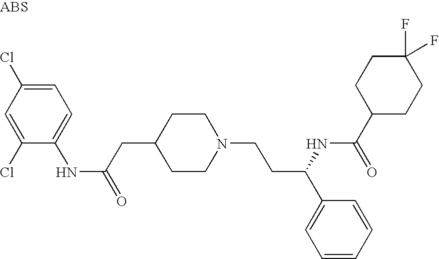 |
| 72 | 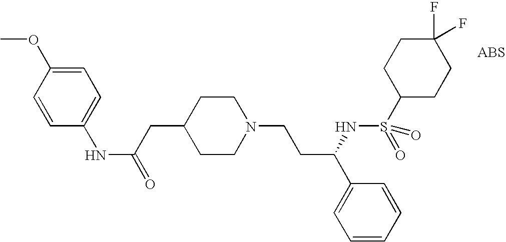 |
| 73 | 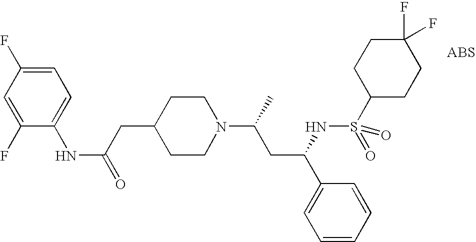 |
| 74 | 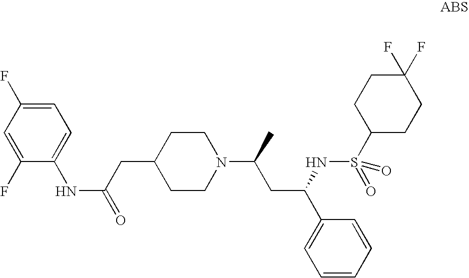 |
| 75 | 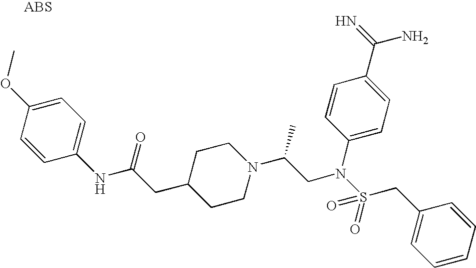 |
| 76 | 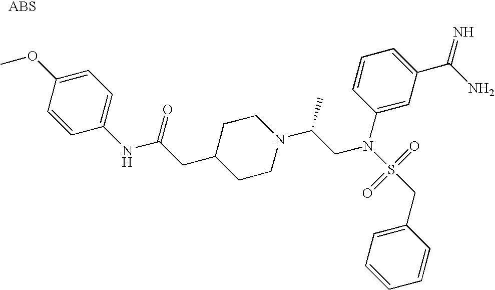 |
| 77 | 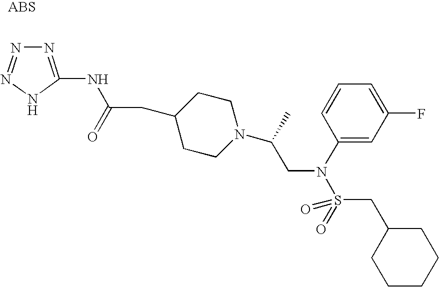 |
| 78 | 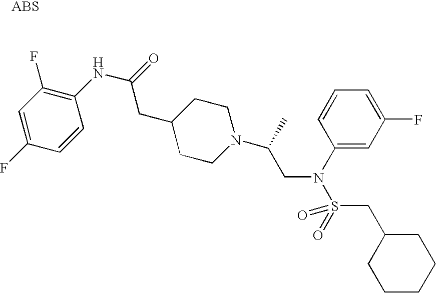 |
| 79 | 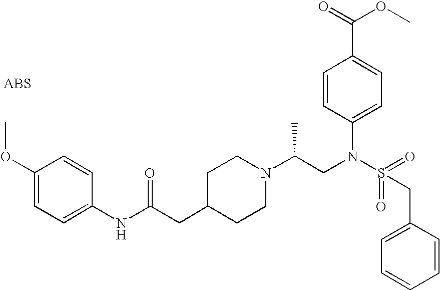 |
| 80 | 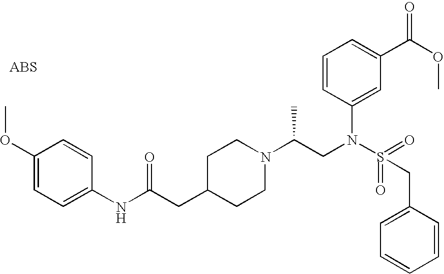 |
| 81 | 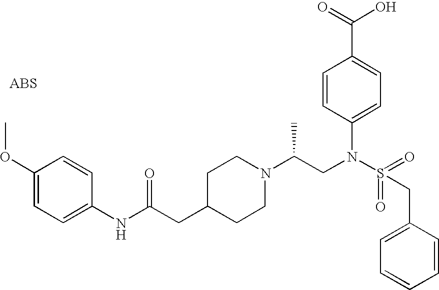 |
| 82 | 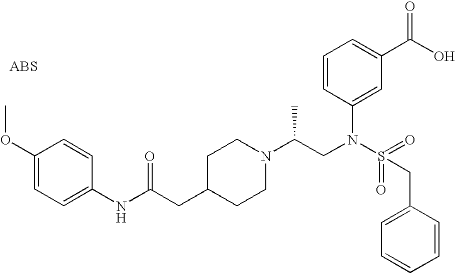 |
| 83 | 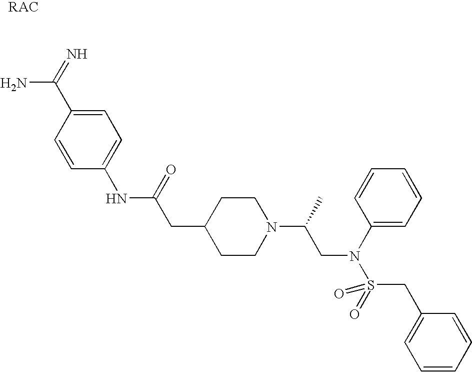 |
| 84 | 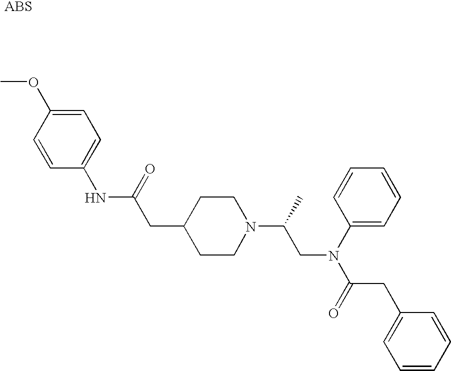 |
| 85 | 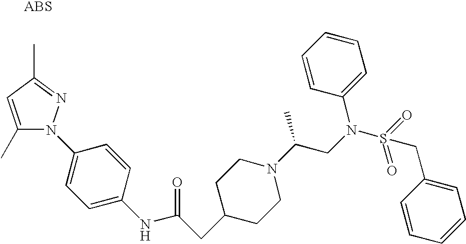 |
| 86 | 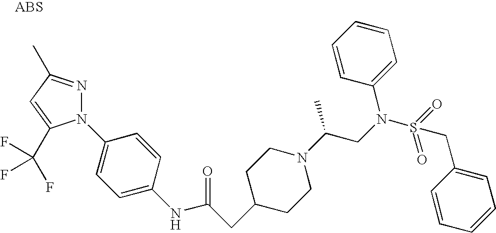 |
| 87 | 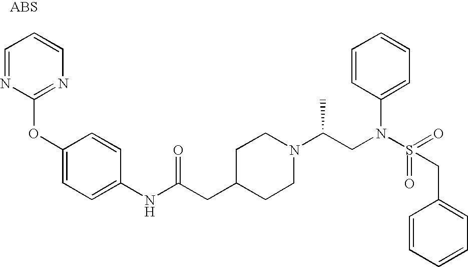 |
| 88 | 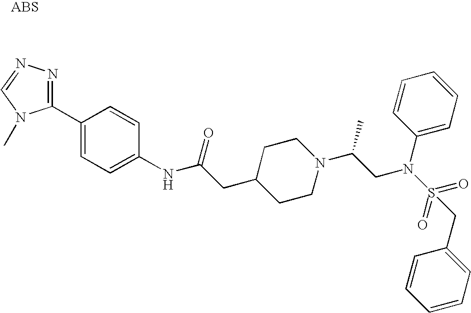 |
| 89 | 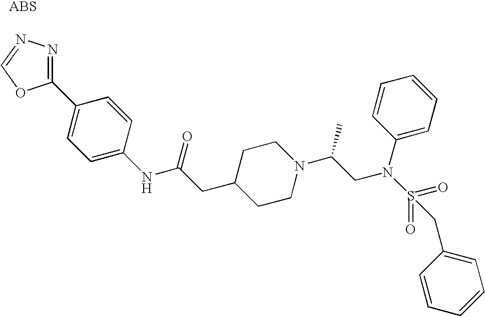 |
| 90 | 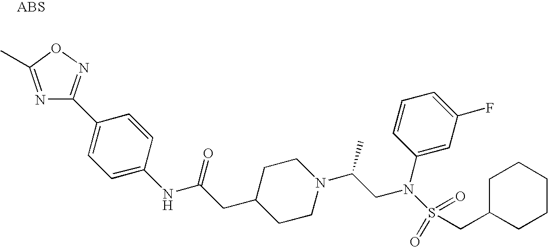 |
| 91 | 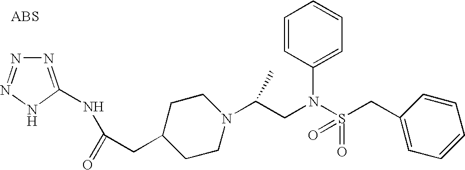 |
| 92 | 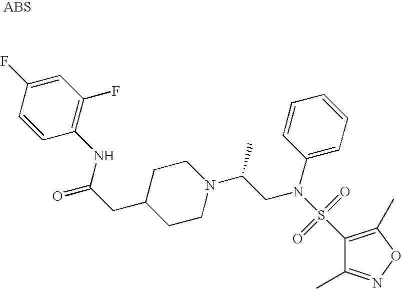 |
| 93 | 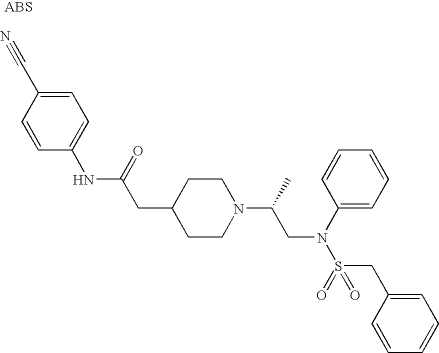 |
| 94 | 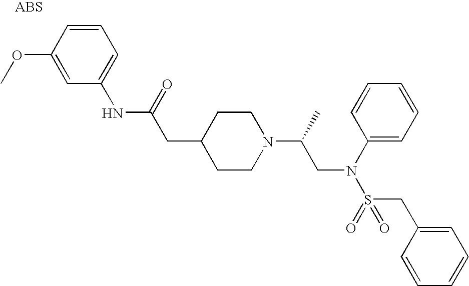 |
| 95 | 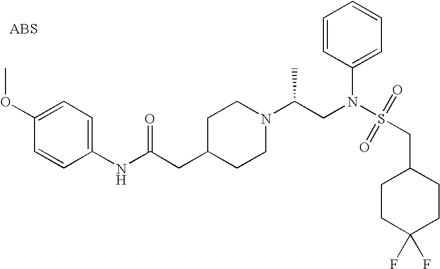 |
| 96 | 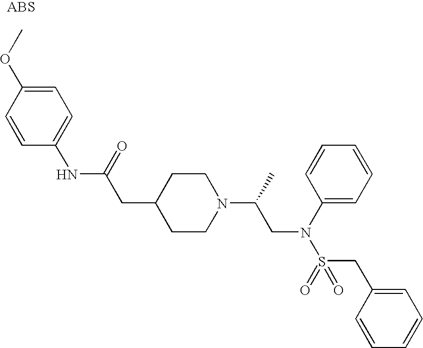 |
| 97 | 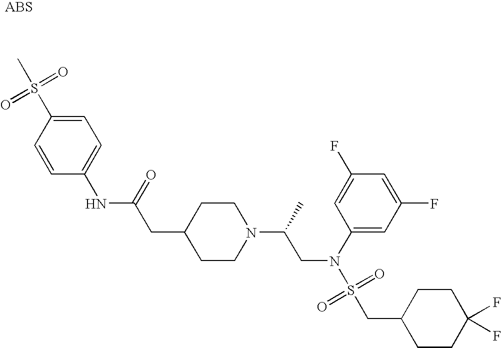 |
| 98 | 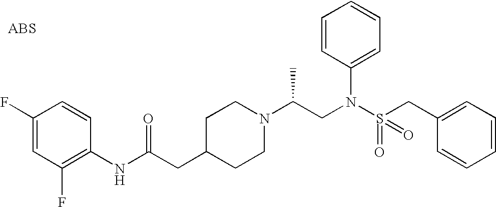 |
| 99 | 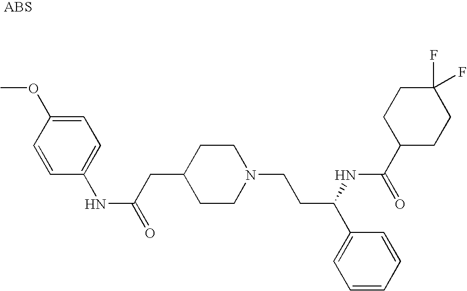 |
| 100 | 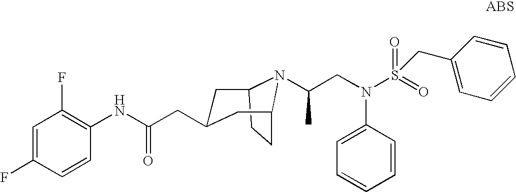 |
| 101 | 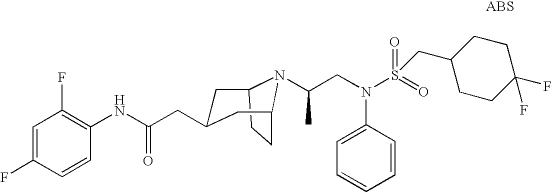 |
| 102 | 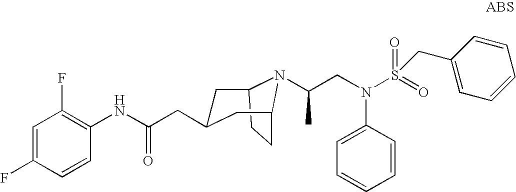 |

### Biology Examples

### Cell Based Assay: Calcium Flux. the Aequorin-Based Assay

The aequorin assay uses the responsiveness of aequorin to intracellular calcium release induced by the activation of G Protein Coupled Receptors (Stables et al., 1997, Anal. Biochem. 252:115-126; Detheux et al., 2000, J. Exp. Med., 192 1501-1508). Briefly, Chinese hamster ovary cells expressing the CCR5 receptor are transfected to coexpress apoaequorin and Gα16. Cells are incubated with 5 μM Coelenterazine H (Promega) overnight at room temperature, and resuspended at a concentration of 0.1×10⁶ cells/ml. Cells are then mixed with test agonist compounds and light emission by the aequorin is recorded with a luminometer (PDSS 6000-Hamamatsu) for 30 sec. Results are expressed as Relative Light Units (RLU). Controls include cells not expressing CCR5 in order to exclude possible non-specific effects of the test compound.

An agonist response is defined as an increase of light emission by aequorin corresponding to 10% or more of the light emitted by a reference sample of cells expressing CCR5 and treated with a the reference agonist ligand MIP-1β. The results of the tested compounds are reported as the concentration of compound required to reach 50% (EC50) of the maximum level of light emission induced by these compounds.

When tested in the assay described above and by way of illustration the compounds no 4, 6, 8, 15, 16, 17 and 27 have an EC50 ranging from 424.5 nM to 4.4 μM (table 11)

### Inhibitory Effect on HIV Infection to MAGI-CCR5 Cells

The inhibitory activity of the compounds of the invention on HIV infection is measured on the human MAGI R5 recombinant cell line coexpressing the human CCR5 receptor and CD4 at their extracellular membrane. MAGI R5 cells are plated in black view plates at 10,000 cells/well and incubated with the appropriate concentrations of the compounds of the invention during 1 hour. This is followed by a 24 hours infection period with the recombinant and non-replicative HIV virus coding for the firefly luciferase (Bona et al., 2006, Antimicrob. Agents Chemother. 50: 3407-3417). The inhibitory effect of the tested compound on virus entry in MAGI R5 cells is measured by a reduction of luciferase signal (TopCount-NXT reader (Packard) and detection luciferase kit: Steadylite HTS assay kit (Perkin Elmer)) in the presence of the compound of the invention relative to the maximum signal obtained from cells infected with the virus without any added compound. The results of the tested compounds are reported as the concentration of compound required to inhibit 50% (IC₅₀) of the maximum luciferase signal.

When tested in the assay described above and by way of illustration the compound nO 50 has an IC₅₀ of 1.6 μM (table 11)

### ¹²⁵I-MIP-1β Binding Competition Assay

The ability of the compounds of the invention to inhibit the binding of MIP-LP was assessed by an in vitro radioligand binding assay. Membranes were prepared from Chinese hamster ovary recombinant cells which express the human CCR5 receptor. The membranes were incubated with 0.05 nM ¹²⁵I-MIP-1β in a HEPES 25 mM/CaCl₂ 5 mM/MgCl₂ 1 mM buffer and various concentrations of the compounds of the invention. The amount of iodinated MIP-1β bound to the receptor was determined after filtration by the quantification of membrane associated radioactivity using the TopCount-NXT reader (Packard). Competition curves were obtained for compounds of the invention and the concentration of compound which displaced 50% of bound radioligand (IC₅₀) was calculated

According to the method described above and by way of illustration the compounds no 4, 6, 8, 15, 16, 17, 27 and 55 have an IC50 (nM) ranging from 13.3 to 436.0 (table 11)

TABLE 11

| | | ¹²⁵I-MIP-1β | |
|---|---|---|---|
| | Aequorin/Ca⁺⁺ | binding | HIV-luciferase |
| | assay | assay | assay |
| ID | EC50 (nM) | IC50 (nM) | IC50 (nM) |
| 50 | +9.5%^{a} | 435.96 | 1603.86 |
| 17 | 437.85 | 134.18 | 55.8%^{b} |
| 8 | 424.53 | 13.34 | 2159.5 |
| 27 | 2326.35 | 167.37 | 31.5%^{b} |
| 16 | 763.99 | 143.31 | 79.6%^{b} |
| 15 | 4412.56 | 105.68 | 1441.4 |
| 4 | 703.22 | 70.82 | 2473.3 |
| 6 | 1763.37 | 115.56 | 2045.6 |
| 98 | 60.5 | 0.4 | 3.6 |
| 96 | 70.6 | 0.4 | 3.9 |
| 69 | 36.4 | Not tested | 2.7 |

^{a}activity level in a range that does not allow the accurate calculation of EC50 value and means level of calcium/aequorin response at a concentration of 10 μM of the compound of the invention compared to the calcium/aequorin response of MIP-1β at 100 nM
^{b}level of inhibition of luciferase activity at a concentration of 10 μM of the compound of the invention compared to the inhibition induced by Rantes at 100 nM

The aequorin-based assay quantitatively determines if the compounds exhibit agonist activity by inducing activation of the CCR5 receptor. The values mentioned in the Table 11 clearly indicate that this is the case. Indeed these values show that the compounds of the invention are able to activate the CCR5 receptor and therefore exhibit agonist activity.

The results of the inhibition of MIP-1β (a reference CCR5 ligand) binding assay represented in table 11 evidences that the compounds of the invention are able to specifically and competitively interact with CCR⁵ receptor.

In addition to the above-mentioned functional and binding activities on the CCR⁵ receptor, the compounds of the invention are also able to protect a human recombinant cell line (MAGI R5 cell) from the infection by a recombinant HIV virus (see table 11, column HIV-Infection assay), which is known to correlate closely with infection of human leukocytes with pathological strains of HIV

In other words the above-mentioned results demonstrate that the compounds of the invention are of value in inhibiting the entry of HIV viruses into target cells and therefore are of value in the prevention of infection by HIV viruses, the treatment of infection by HIV viruses and the prevention and/or the treatment of acquired immune deficiency syndrome (AIDS).

1. A compound of general Formula I: 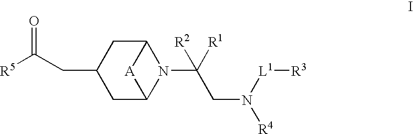
and pharmaceutically acceptable salts and solvates thereof, wherein
A is —CH₂—CH₂— or absent;
R¹ and R² independently are H, halo, optionally substituted alkyl, aryl, heteroaryl, cycloalkyl, cycloalkylalkyl, or heterocyclyl;
R³ and R⁴ independently are a group selected from aryl, heteroaryl, cycloalkyl, and heterocyclyl, each group being optionally substituted by one or more substituent(s) selected from halo, oxo, nitro, cyano, azido, alkyl, hydroxyalkyl, haloalkyl, cycloalkyl, cycloalkylalkyl, alkenyl, alkynyl, heteroalkyl, heterocyclyl, heterocyclylalkyl, aryl, aralkyl, heteroaryl, heteroarylalkyl, hydroxyl, alkoxy, haloalkoxy, cycloalkyloxy, heterocyclyloxy, aryloxy, thiol, alkylthio, thioalkyl, haloalkylthio, acyl, thioacyl, aroyl, amino, alkylamino, aminoalkyl, carboxy, alkoxycarbonyl, cycloalkyloxycarbonyl, heterocyclyloxycarbonyl, aryloxycarbonyl, heteroaryloxycarbonyl, alkylcarbonyloxy, cycloalkylcarbonyloxy, heterocyclylcarbonyloxy, arylcarbonyloxy, heteroarylcarbonyloxy, arylalkyloxy, alkylcarbonylamino, haloalkylcarbonylamino, cycloalkylcarbonylamino, heterocyclylcarbonylamino arylcarbonylamino, heteroarylcarbonylamino, alkylcarbonylaminoalkyl, acylamino, carbamoyl, hydroxycarbamoyl, alkylcarbamoyl, arylcarbamoyl, heteroarylcarbamoyl, carbamoylalkyl, carbamoylamino, alkylcarbamoylamino, sulfino, alkylsulfinyl, sulfo, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, and haloalkylsulfonylamino, or two substituents form an alkylenedioxy group or a haloalkylenedioxy group, or fused to the cycloalkyl, aryl, or heterocyclyl group may be one or more cycloalkyl, aryl, heterocyclyl or heteroaryl group, each of said groups being optionally substituted by one or more further substituent(s) selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, nitro, oxo, and sulfonyl;
L¹ is NRCO, NRSO₂, CO, CONR, CONRCH₂, CH₂CO, COCH₂ CH₂CH₂CO, CH₂COCH₂, COCH₂CH₂, SO₂, SO₂NR, SO₂CH₂, SO₂CH₂CH₂, a single bond or a group selected from C₁-C₃ alkylene, C₂-C₄ alkenylene and C₂-C₄ alkynylene, each group being optionally substituted with one or more substituent(s) selected from alkyl, aryl, heteroaryl, halo, alkylcarbonyl, alkylamino, alkoxy, alkylcarbonylamino, and alkylcarbonylalkyl, wherein R is hydrogen or C₁-C₆ alkyl;
R⁵ is selected from NR⁶(L²-R⁸), O (L²-R⁸), and CR⁶R⁷(L²-R⁸);
R⁶ and R⁷ independently are selected from hydrogen, C₁-C₄ alkyl, allyl, propargyl, —CH₂—CH₂—OH, —CH₂—CH₂—CH₂—OH, cyclopropyl, cyclopropylmethyl, aryl, and heteroaryl;
L² is a single bond or C₁-C₄ alkylene, optionally substituted by one or more substituent(s) selected from halo, oxo, nitro, cyano, azido, alkyl, hydroxyalkyl, haloalkyl, cycloalkyl, cycloalkylalkyl, and alkoxy, or L² is CR^{a}R^{b}, wherein R^{a} and R^{b} form together with the carbon to which they are attached a carbocycle having 3 to 6 ring atoms;
R⁸ is a group selected from aryl, heteroaryl, cycloalkyl, and heterocyclyl, each group being optionally substituted by one or more substituent(s) selected from halo, oxo, nitro, cyano, azido, alkyl, hydroxyalkyl, haloalkyl, cycloalkyl, cycloalkylalkyl, alkenyl, alkynyl, heteroalkyl, heterocyclyl, heterocyclylalkyl, aryl, aralkyl, heteroaryl, heteroarylalkyl, hydroxyl, alkoxy, haloalkoxy, cycloalkyloxy, heterocyclyloxy, aryloxy, thiol, alkylthio, thioalkyl, haloalkylthio, acyl, thioacyl, aroyl, amino, alkylamino, aminoalkyl, carboxy, alkoxycarbonyl, cycloalkyloxycarbonyl, heterocyclyloxycarbonyl, aryloxycarbonyl, heteroaryloxycarbonyl, alkylcarbonyloxy, cycloalkylcarbonyloxy, heterocyclylcarbonyloxy, arylcarbonyloxy, heteroarylcarbonyloxy, arylalkyloxy, alkylcarbonylamino, haloalkylcarbonylamino, cycloalkylcarbonylamino, heterocyclylcarbonylamino arylcarbonylamino, heteroarylcarbonylamino, alkylcarbonylaminoalkyl acylamino, carbamoyl, hydroxycarbamoyl, alkylcarbamoyl, arylcarbamoyl, heteroarylcarbamoyl, carbamoylalkyl, carbamoylamino, alkylcarbamoylamino, sulfino, alkylsulfinyl, sulfo, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, and haloalkylsulfonylamino, or two substituents form an alkylenedioxy group or a haloalkylenedioxy group, or fused to the cycloalkyl, aryl, or heterocyclyl group may be one or more cycloalkyl, aryl, heterocyclyl or heteroaryl group, each of said groups being optionally substituted by one or more further substituent(s) selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, nitro, oxo, and sulfonyl, or
R⁶ and L²-R⁸ form together with the nitrogen atom to which they are connected a 5 to 8 membered saturated, or unsaturated cycle, which cycle is optionally substituted by one or more groups selected from aryl, heteroaryl, cycloalkyl, and heterocyclyl, each group being optionally substituted by one or more substituent(s) selected from halo, oxo, nitro, cyano, azido, alkyl, hydroxyalkyl, haloalkyl, cycloalkyl, cycloalkylalkyl, alkenyl, alkynyl, heteroalkyl, heterocyclyl, heterocyclylalkyl, aryl, aralkyl, heteroaryl, heteroarylalkyl, hydroxyl, alkoxy, haloalkoxy, cycloalkyloxy, heterocyclyloxy, aryloxy, thiol, alkylthio, thioalkyl, haloalkylthio, acyl, thioacyl, aroyl, amino, alkylamino, aminoalkyl, carboxy, alkoxycarbonyl, cycloalkyloxycarbonyl, heterocyclyloxycarbonyl, aryloxycarbonyl, heteroaryloxycarbonyl, alkylcarbonyloxy, cycloalkylcarbonyloxy, heterocyclylcarbonyloxy, arylcarbonyloxy, heteroarylcarbonyloxy, arylalkyloxy, alkylcarbonylamino, haloalkylcarbonylamino, cycloalkylcarbonylamino, heterocyclylcarbonylamino arylcarbonylamino, heteroarylcarbonylamino, alkylcarbonylaminoalkyl, acylamino, carbamoyl, hydroxycarbamoyl, alkylcarbamoyl, arylcarbamoyl, heteroarylcarbamoyl, carbamoylalkyl, carbamoylamino, alkylcarbamoylamino, sulfino, alkylsulfinyl, sulfo, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, and haloalkylsulfonylamino, or two substituents form an alkylenedioxy group or a haloalkylenedioxy group, or fused to the 5 to 8 membered saturated, or unsaturated cycle may be one or more cycloalkyl, aryl, heterocyclyl or heteroaryl group, each of said groups being optionally substituted by one or more further substituent(s) selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, nitro, oxo, and sulfonyl, or
R⁶ and L²-R⁸ form together with the carbon atom to which they are connected a 5 to 8 membered saturated, partially unsaturated or aromatic cycle, which cycle is optionally substituted by one or more groups selected from aryl, heteroaryl, cycloalkyl, and heterocyclyl, each group being optionally substituted by one or more substituent(s) selected from halo, oxo, nitro, cyano, azido, alkyl, hydroxyalkyl, haloalkyl, cycloalkyl, cycloalkylalkyl, alkenyl, alkynyl, heteroalkyl, heterocyclyl, heterocyclylalkyl, aryl, aralkyl, heteroaryl, heteroarylalkyl, hydroxyl, alkoxy, haloalkoxy, cycloalkyloxy, heterocyclyloxy, aryloxy, thiol, alkylthio, thioalkyl, haloalkylthio, acyl, thioacyl, aroyl, amino, alkylamino, aminoalkyl, carboxy, alkoxycarbonyl, cycloalkyloxycarbonyl, heterocyclyloxycarbonyl, aryloxycarbonyl, heteroaryloxycarbonyl, alkylcarbonyloxy, cycloalkylcarbonyloxy, heterocyclylcarbonyloxy, arylcarbonyloxy, heteroarylcarbonyloxy, arylalkyloxy, alkylcarbonylamino, haloalkylcarbonylamino, cycloalkylcarbonylamino, heterocyclylcarbonylamino arylcatbonylamino, heteroarylcarbonylamino, alkylcarbonylaminoalkyl, acylamino, carbamoyl, hydroxycarbamoyl, alkylcarbamoyl, arylcarbamoyl, heteroarylcarbamoyl, carbamoylalkyl, carbamoylamino, alkylcarbamoylamino, sulfino, alkylsulfinyl, sulfo, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, and haloalkylsulfonylamino, or two substituents form an alkylenedioxy group or a haloalkylenedioxy group, or fused to the 5 to 8 membered saturated, partially unsaturated or aromatic cycle may be one or more cycloalkyl, aryl, heterocyclyl or heteroaryl group, each of said groups being optionally substituted by one or more further substituent(s) selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, nitro, oxo, and sulfonyl.

2. A compound according to claim 1 or a pharmaceutically acceptable salt or solvate thereof, wherein
A is absent;
R¹ and R² independently are hydrogen, or C₁-C₄ alkyl; preferably hydrogen or methyl;
L¹, R³, R⁴ and R⁸ are as defined in claim 1;
R¹ is NR⁶(L²-R⁸);
R⁶ is selected from hydrogen, C₁-C₄ alkyl, allyl, propargyl, —CH₂—CH₂—OH, —CH₂—CH₂—CH₂—OH, cyclopropyl, cyclopropylmethyl, aryl, and heteroaryl; preferably hydrogen or C₁-C₄ alkyl; most preferably hydrogen; and
L² is a single bond.

3. A compound according to claim 1 or a pharmaceutically acceptable salt or solvate thereof, wherein
R³ is defined as in claim 1;
A is absent;
R¹ is hydrogen;
R² is hydrogen or methyl, preferably methyl;
R⁴ is aryl, optionally substituted by one or more substituent(s) selected from halo, oxo, nitro, cyano, azido, alkyl, hydroxyalkyl, haloalkyl, cycloalkyl, cycloalkylalkyl, alkenyl, alkynyl, heteroalkyl, heterocyclyl, heterocyclylalkyl, aryl, aralkyl, heteroaryl, heteroarylalkyl, hydroxyl, alkoxy, haloalkoxy, cycloalkyloxy, heterocyclyloxy, aryloxy, thiol, alkylthio, thioalkyl, haloalkylthio, acyl, thioacyl, aroyl, amino, alkylamino, aminoalkyl, carboxy, alkoxycarbonyl, cycloalkyloxycarbonyl, heterocyclyloxycarbonyl, aryloxycarbonyl, heteroaryloxycarbonyl, alkylcarbonyloxy, cycloalkylcarbonyloxy, heterocyclylcarbonyloxy, arylcarbonyloxy, heteroarylcarbonyloxy, arylalkyloxy, alkylcarbonylamino, haloalkylcarbonylamino, cycloalkylcarbonylamino, heterocyclylcarbonylamino arylcarbonylamino, heteroarylcarbonylamino, alkylcarbonylaminoalkyl, acylamino, carbamoyl, hydroxycarbamoyl, alkylcarbamoyl, arylcarbamoyl, heteroarylcarbamoyl, carbamoylalkyl, carbamoylamino, alkylcarbamoylamino, sulfino, alkylsulfinyl, sulfo, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, and haloalkylsulfonylamino, or two substituents form an alkylenedioxy group or a haloalkylenedioxy group, or fused to the aryl group may be one or more cycloalkyl, aryl, heterocyclyl or heteroaryl group, each of said substituents being optionally substituted by one or more further substituent(s) selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, nitro, oxo, and sulfonyl;
L¹ is as defined in claim 1;
R⁵ is NR⁶(L²-R⁸);
R⁶ is hydrogen;
L² is a single bond; and
R⁸ is as defined in claim 1.

4. A compound according to claim 1 having Formula Ia: 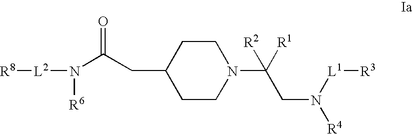
and pharmaceutically acceptable salts and solvates thereof, wherein R¹, R², R³, and R⁴, L¹, R⁶, L² and R⁸ are as defined in claim 1.

5. A compound according to claim 4 having the Formula Ia, wherein
L¹ is CO, CONH, CONHCH₂, CH₂CO, COCH₂ CH₂CH₂CO, CH₂COCH₂, COCH₂CH₂, SO₂NH, SO₂CH₂, SO₂CH₂CH₂, a single bond or a group selected from C₁-C₃ alkylene, C₂-C₄ alkenylene and C₂-C₄ alkynylene, each group being optionally substituted with one or more substituent(s) selected from alkyl, aryl, heteroaryl, halo, alkylcarbonyl, alkylamino, alkoxy, alkylcarbonylamino, and alkylcarbonylalkyl.

6. A compound according to claim 1 having Formula Ib: 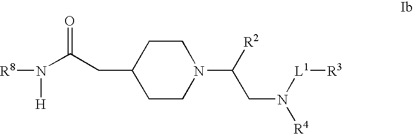
and pharmaceutically acceptable salts and solvates thereof, wherein
R² is H, or C₁-C₄ alkyl;
R³ and R⁴ are as defined in claim 1;
L¹ is CO, CONH, CONHCH₂, CH₂CO, COCH₂ CH₂CH₂CO, CH₂COCH₂. COCH₂CH₂, SO₂NH, SO₂CH₂, SO₂, SO₂CH₂CH₂, a single bond or a group selected from C₁-C₃ alkylene, C₂-C₄ alkenylene and C₂-C₄ alkynylene, each group being optionally substituted with one or more substituent(s) selected from alkyl, aryl, heteroaryl, halo, alkylcarbonyl, alkylamino, alkoxy, alkylcarbonylamino, and alkylcarbonylalkyl; and
R⁸ is a group selected from aryl, heteroaryl cycloalkyl, and heterocyclyl, each group being optionally substituted by one or more substituent(s) selected from halo, oxo, nitro, cyano, azido, alkyl, hydroxyalkyl, haloalkyl, cycloalkyl, cycloalkylalkyl, alkenyl, alkynyl, heteroalkyl, heterocyclyl, heterocyclylalkyl, aryl, aralkyl, heteroaryl, heteroarylalkyl, hydroxyl, alkoxy, haloalkoxy, cycloalkyloxy, heterocyclyloxy, aryloxy, thiol, alkylthio, thioalkyl, haloalkylthio, acyl, thioacyl, aroyl, amino, alkylamino, aminoalkyl, carboxy, alkoxycarbonyl, cycloalkyloxycarbonyl, heterocyclyloxycarbonyl, aryloxycarbonyl, heteroaryloxycarbonyl, alkylcarbonyloxy, cycloalkylcarbonyloxy, heterocyclylcarbonyloxy, arylcarbonyloxy, heteroarylcarbonyloxy, arylalkyloxy, alkylcarbonylamino, haloalkylcarbonylamino, cycloalkylcarbonylamino, heterocyclylcarbonylamino arylcarbonylamino, heteroarylcarbonylamino, alkylcarbonylaminoalkyl, acylamino, carbamoyl, hydroxycarbamoyl, alkylcarbamoyl, arylcarbamoyl, heteroarylcarbamoyl, carbamoylalkyl, carbamoylamino, alkylcarbamoylamino, sulfino, alkylsulfinyl, sulfo, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, and haloalkylsulfonylamino, or two substituents form an alkylenedioxy group or a haloalkylenedioxy group, or fused to the cycloalkyl, aryl, or heterocyclyl group may be one or more cycloalkyl, aryl, heterocyclyl or heteroaryl groups, each of said groups being optionally substituted by one or more further substituent(s) selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, nitro, oxo, and sulfonyl.

7. A compound according to claim 1 having Formula Ic: 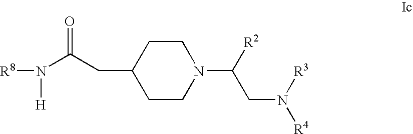
and pharmaceutically acceptable salts and solvates thereof, wherein
R² is H, or C₁-C₄ alkyl;
R³ and R⁴ are aryl, independently optionally substituted by one or more substituent(s) selected from halo, oxo, nitro, cyano, azido, alkyl, hydroxyalkyl, haloalkyl, cycloalkyl, cycloalkylalkyl, alkenyl, alkynyl, heteroalkyl, heterocyclyl, heterocyclylalkyl, aryl, aralkyl, heteroaryl, heteroarylalkyl, hydroxyl, alkoxy, haloalkoxy, cycloalkyloxy, heterocyclyloxy, aryloxy, thiol, alkylthio, thioalkyl, haloalkylthio, acyl, thioacyl, aroyl, amino, alkylamino, aminoalkyl, carboxy, alkoxycarbonyl, cycloalkyloxycarbonyl, heterocyclyloxycarbonyl, aryloxycarbonyl, heteroaryloxycarbonyl, alkylcarbonyloxy, cycloalkylcarbonyloxy, heterocyclylcarbonyloxy, arylcarbonyloxy, heteroarylcarbonyloxy, arylalkyloxy, alkylcarbonylamino, haloalkylcarbonylamino, cycloalkylcarbonylamino, heterocyclylcarbonylamino arylcarbonylamino, heteroarylcarbonylamino, alkylcarbonylaminoalkyl, acylamino, carbamoyl, hydroxycarbamoyl, alkylcarbamoyl, arylcarbamoyl, heteroarylcarbamoyl, carbamoylalkyl, carbamoylamino, alkylcarbamoylamino, sulfino, alkylsulfinyl, sulfo, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, and haloalkylsulfonylamino, or two substituents form an alkylenedioxy group or a haloalkylenedioxy group, or fused to the aryl group may be one or more cycloalkyl, aryl, heterocyclyl or heteroaryl group, each of said groups being optionally substituted by one or more further substituent(s) selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, nitro, oxo, and sulfonyl;
R⁸ is aryl, optionally substituted by one or more substituent(s) selected from halo, oxo, nitro, cyano, azido, alkyl, hydroxyalkyl, haloalkyl, cycloalkyl, cycloalkylalkyl, alkenyl, alkynyl, heteroalkyl, heterocyclyl, heterocyclylalkyl, aryl, aralkyl, heteroaryl, heteroarylalkyl, hydroxyl, alkoxy, haloalkoxy, cycloalkyloxy, heterocyclyloxy, aryloxy, thiol, alkylthio, thioalkyl, haloalkylthio, acyl, thioacyl, aroyl, amino, alkylamino, aminoalkyl, carboxy, alkoxycarbonyl, cycloalkyloxycarbonyl, heterocyclyloxycarbonyl, aryloxycarbonyl, heteroaryloxycarbonyl, alkylcarbonyloxy, cycloalkylcarbonyloxy, heterocyclylcarbonyloxy, arylcarbonyloxy, heteroarylcarbonyloxy, arylalkyloxy, alkylcarbonylamino, haloalkylcarbonylamino, cycloalkylcarbonylamino, heterocyclylcarbonylamino arylcarbonylamino, heteroarylcarbonylamino, alkylcarbonylaminoalkyl, acylamino, carbamoyl, hydroxycarbamoyl, alkylcarbamoyl, arylcarbamoyl, heteroarylcarbamoyl, carbamoylalkyl, carbamoylamino, alkylcarbamoylamino, sulfino, alkylsulfinyl, sulfo, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, and haloalkylsulfonylamino, or two substituents form an alkylenedioxy group or a haloalkylenedioxy group, or fused to the aryl group may be one or more cycloalkyl, aryl, heterocyclyl or heteroaryl group, each of said groups being optionally substituted by one or more further substituent(s) selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, nitro, oxo, and sulfonyl.

8. The compound according to claim 7 or a pharmaceutically acceptable salt or solvate thereof, wherein
R³ and R⁴ are phenyl, independently optionally substituted by one or more substituent(s) selected from halo, oxo, nitro, cyano, azido, alkyl, hydroxyalkyl, haloalkyl, cycloalkyl, cycloalkylalkyl, alkenyl, alkynyl, heteroalkyl, heterocyclyl, heterocyclylalkyl, aryl, aralkyl, heteroaryl, heteroarylalkyl, hydroxyl, alkoxy, haloalkoxy, cycloalkyloxy, heterocyclyloxy, aryloxy, thiol, alkylthio, thioalkyl, haloalkylthio, acyl, thioacyl, aroyl, amino, alkylamino, aminoalkyl, carboxy, alkoxycarbonyl, cycloalkyloxycarbonyl, heterocyclyloxycarbonyl, aryloxycarbonyl, heteroaryloxycarbonyl, alkylcarbonyloxy, cycloalkylcarbonyloxy, heterocyclylcarbonyloxy, arylcarbonyloxy, heteroarylcarbonyloxy, arylalkyloxy, alkylcarbonylamino, haloalkylcarbonylamino, cycloalkylcarbonylamino, heterocyclylcarbonylamino arylcarbonylamino, heteroarylcarbonylamino, alkylcarbonylaminoalkyl, acylamino, carbamoyl, hydroxycarbamoyl, alkylcarbamoyl, arylcarbamoyl, heteroarylcarbamoyl, carbamoylalkyl, carbamoylamino, alkylcarbamoylamino, sulfino, allylsulfinyl, sulfo, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, haloalkylsulfonylamino, or two substituents form an alkylenedioxy group or a haloalkylenedioxy group, or fused to the phenyl group may be one or more cycloalkyl, aryl, heterocyclyl or heteroaryl group, each of said groups being optionally substituted by one or more further substituent(s) selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, nitro, oxo, and sulfonyl.

9. A compound according to claim 7 or a pharmaceutically acceptable salt or solvate thereof, wherein
R⁸ is phenyl, optionally substituted by one or more substituent(s) selected from halo, oxo, nitro, cyano, azido, alkyl, hydroxyalkyl, haloalkyl, cycloalkyl, cycloalkylalkyl, alkenyl, alkynyl, heteroalkyl, heterocyclyl, heterocyclylalkyl, aryl, aralkyl, heteroaryl, heteroarylalkyl, hydroxyl, alkoxy, haloalkoxy, cycloalkyloxy, heterocyclyloxy, aryloxy, thiol, alkylthio, thioalkyl, haloalkylthio, acyl, thioacyl, aroyl, amino, alkylamino, aminoalkyl, carboxy, alkoxycarbonyl, cycloalkyloxycarbonyl, heterocyclyloxycarbonyl, aryloxycarbonyl, heteroaryloxycarbonyl, alkylcarbonyloxy, cycloalkylcarbonyloxy, heterocyclylcarbonyloxy, arylcarbonyloxy, heteroarylcarbonyloxy, arylalkyloxy, alkylcarbonylamino, haloalkylcarbonylamino, cycloalkylcarbonylamino, heterocyclylcarbonylamino arylcarbonylamino, heteroarylcarbonylamino, alkylcarbonylaminoalkyl, acylamino, carbamoyl, hydroxycarbamoyl, alkylcarbamoyl, arylcarbamoyl, heteroarylcarbamoyl, carbamoylalkyl, carbamoylamino, alkylcarbamoylamino, sulfino, alkylsulfinyl, sulfo, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, and haloalkylsulfonylamino, or two substituents form an alkylenedioxy group or a haloalkylenedioxy group, or fused to the phenyl group may be one or more cycloalkyl, aryl, heterocyclyl or heteroaryl group, each of said groups being optionally substituted by one or more further substituent(s) selected from halo, alkoxy, alkyl, alkylamino, alkylcarbonyl, alkylheteroaryl, alkylsulfonyl, aralkyl, aryl, arylamino, aryloxy, cyano, haloalkoxy, haloalkyl, heteroaryl, heteroarylalkyl, heteroarylcarbonyl, heterocyclyl, hydroxyl, nitro, oxo, and sulfonyl.

10. A compound according to claim 1 having Formula Id: 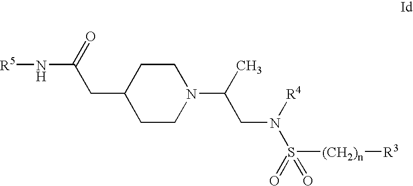
and pharmaceutically acceptable salts and solvates thereof, wherein
n is 0, 1 or 2;
R³ is aryl, heteroaryl or cycloalkyl, optionally substituted by one or more substituent(s) selected from halo, oxo, nitro, cyano, azido, alkyl, hydroxyalkyl, cycloalkyl, alkynyl, hydroxyl, alkoxy, haloalkoxy, thiol, alkylthio, thioalkyl, haloalkylthio, acyl, thioacyl, amino, alkylamino, aminoalkyl, carboxy, alkoxycarbonyl, cycloalkyloxycarbonyl, alkylcarbonyloxy, alkylcarbonylamino, haloalkylcarbonylamino, cycloalkylcarbonylamino, alkylcarbonylaminoalkyl, acylamino, carbamoyl, hydroxycarbamoyl, alkylcarbamoyl, carbamoylalkyl, carbamoylamino, alkylcarbamoylamino, sulfino, alkylsulfinyl, sulfo, alkylsulfonyl, haloalkylsulfonyl, cycloalkylsulfonyl, heterocyclylsulfonyl, arylsulfonyl, heteroarylsulfonyl sulfamoyl, alkylsulfamoyl, arylsulfamoyl, heteroarylsulfamoyl, alkylsulfonylamino, cycloalkylsulfonylamino, heterocyclylsulfonylamino, arylsulfonylamino, heteroarylsulfonylamino, and haloalkylsulfonylamino, or two substituents form an alkylenedioxy group or a haloalkylenedioxy group, or fused to the phenyl or pyridinyl group may be one or more cycloalkyl, aryl, heterocyclyl or heteroaryl group;
R⁴ is defined as in claim 1; and
R⁵ is defined as in claim 1.

11. A compound according to claim 10 having formula Id′ 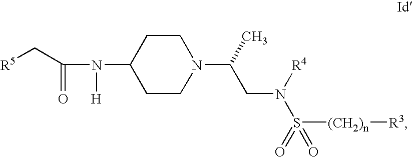
and pharmaceutically acceptable salts and solvates thereof, wherein n, R³, R⁴, and R⁵ are defined as in claim 10.

12. A compound according to claim 1, selected from the group consisting of 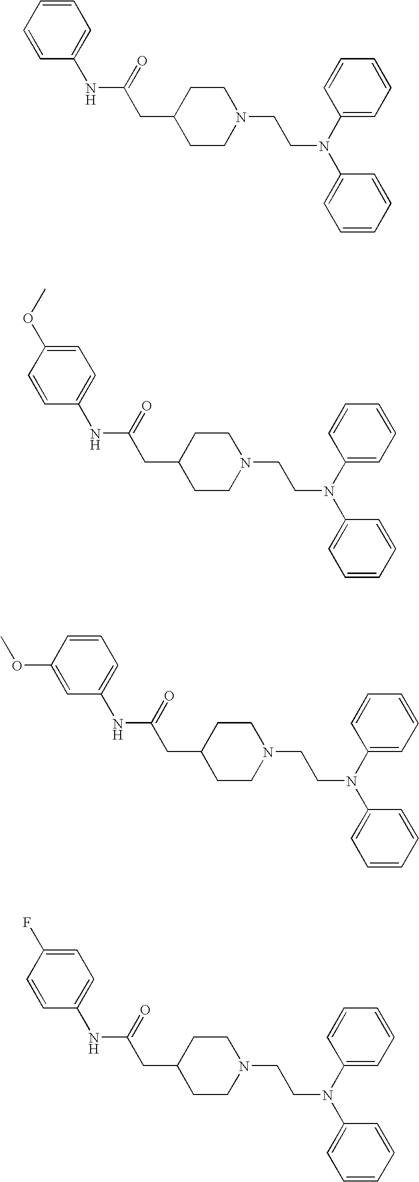 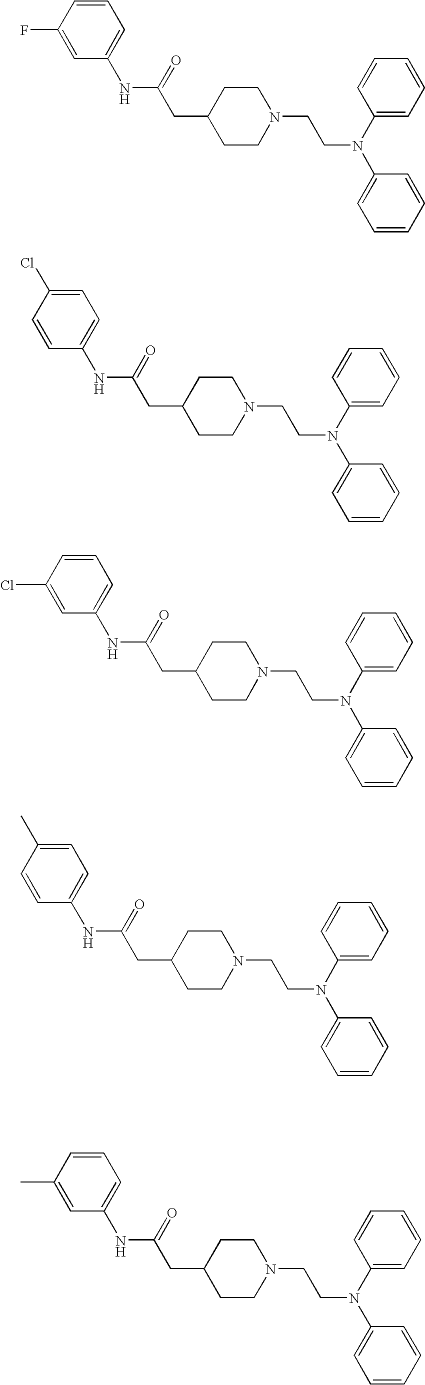 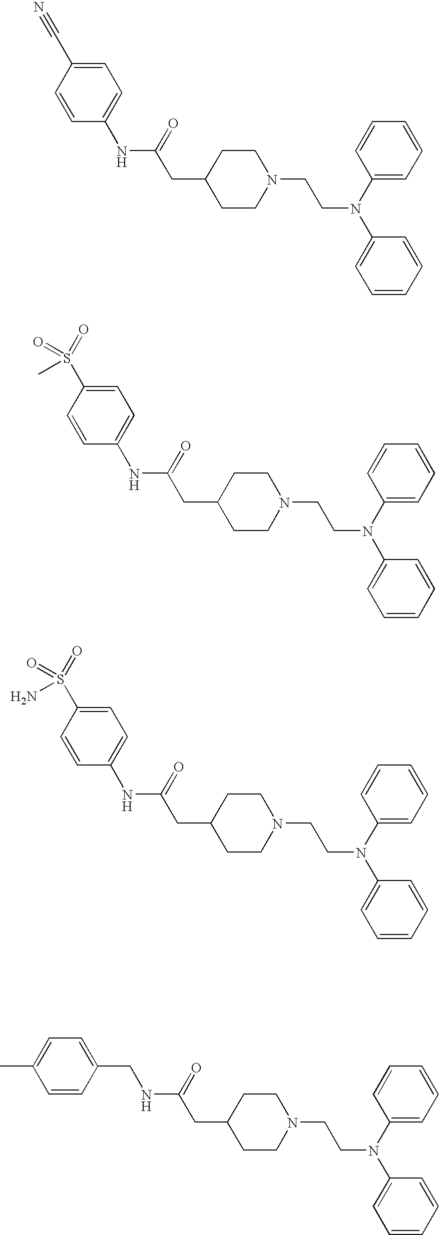 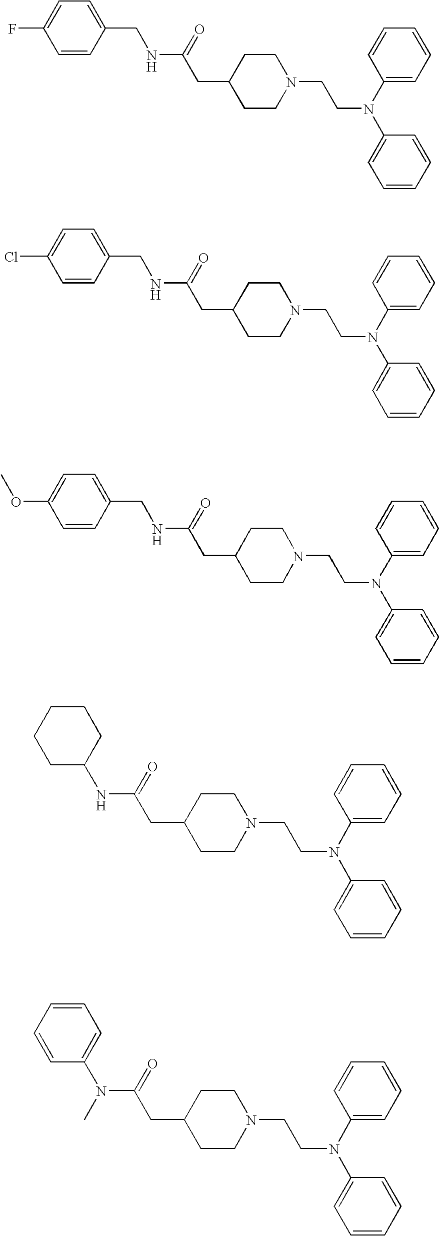 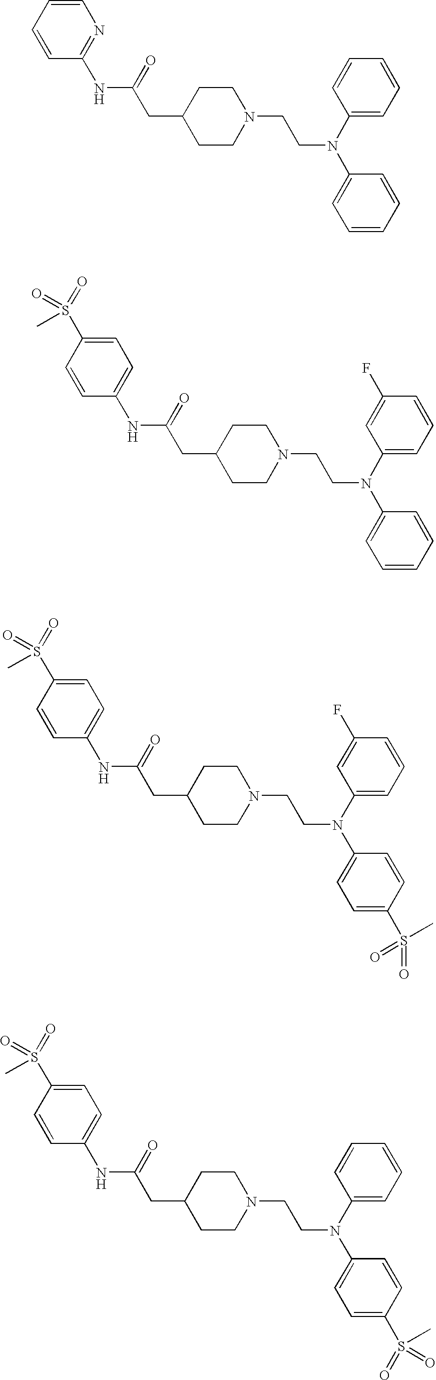 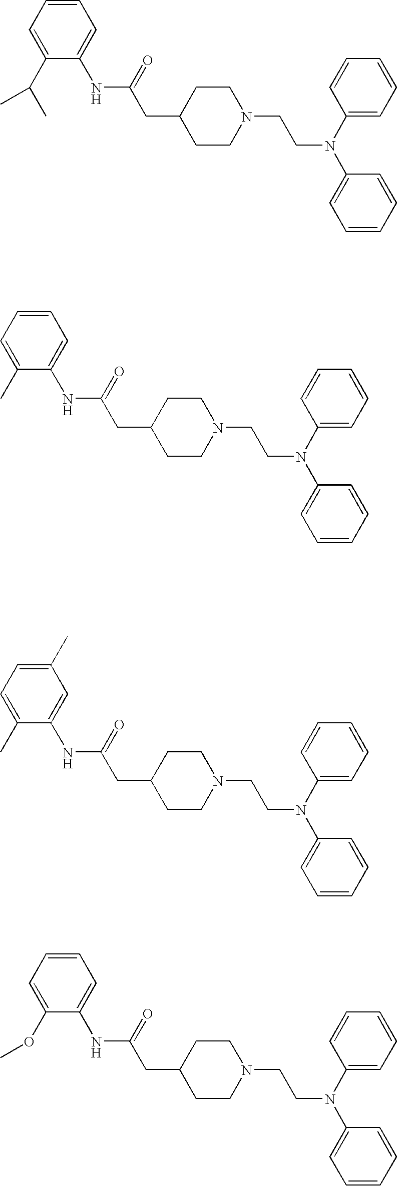 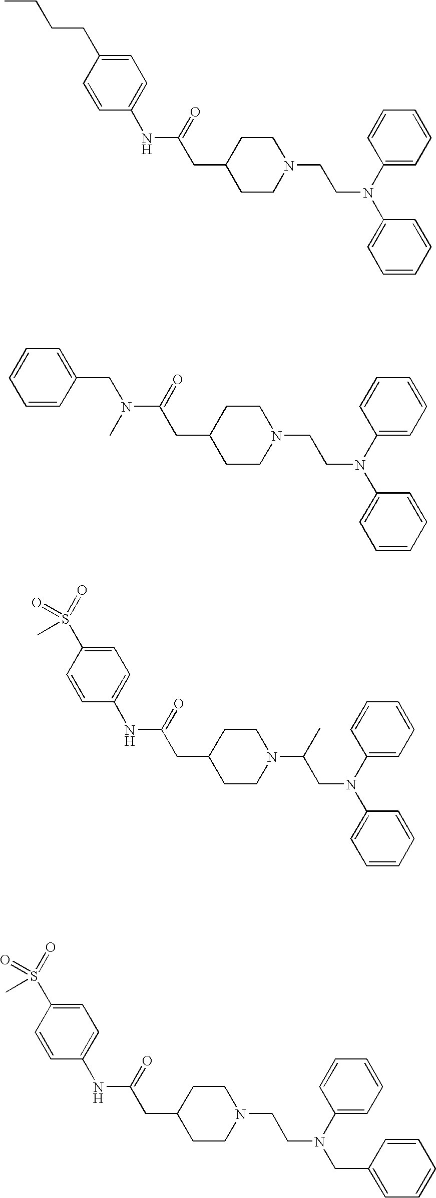 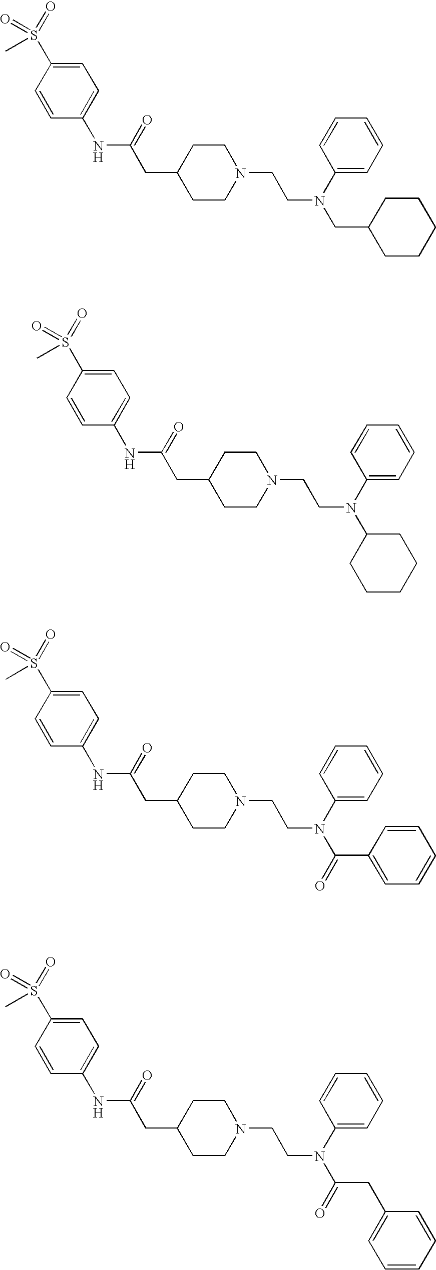 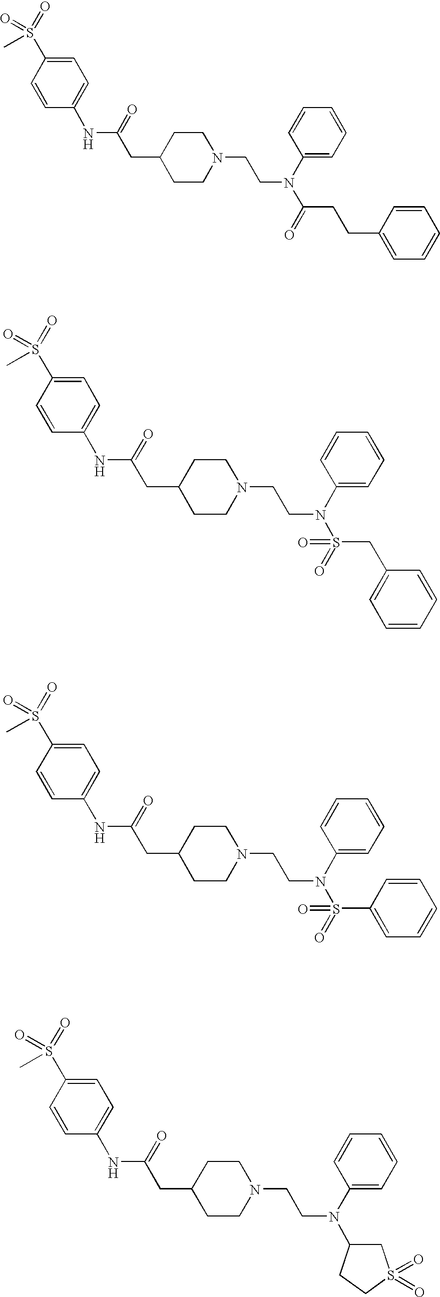 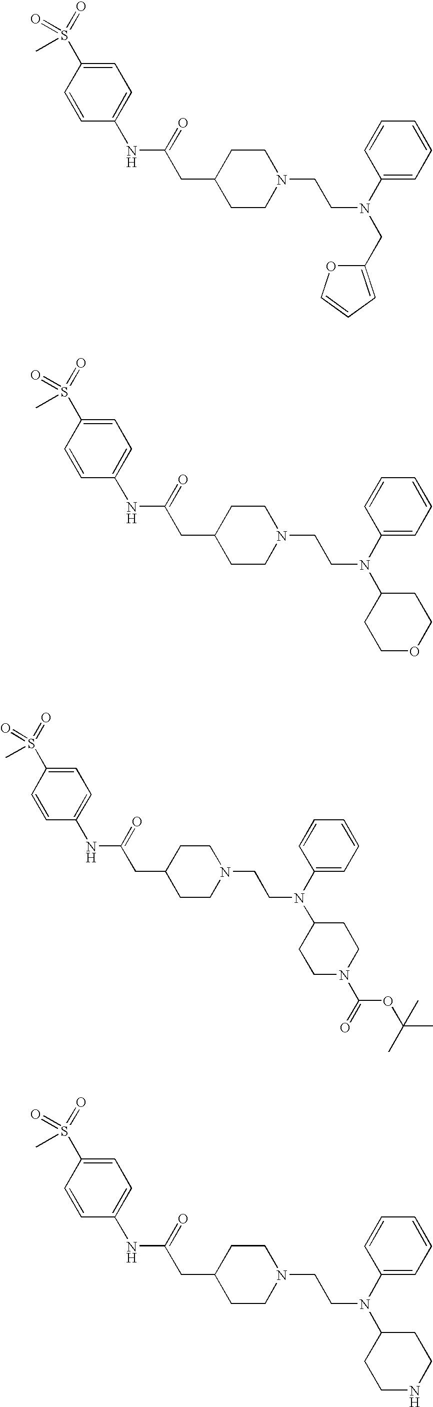 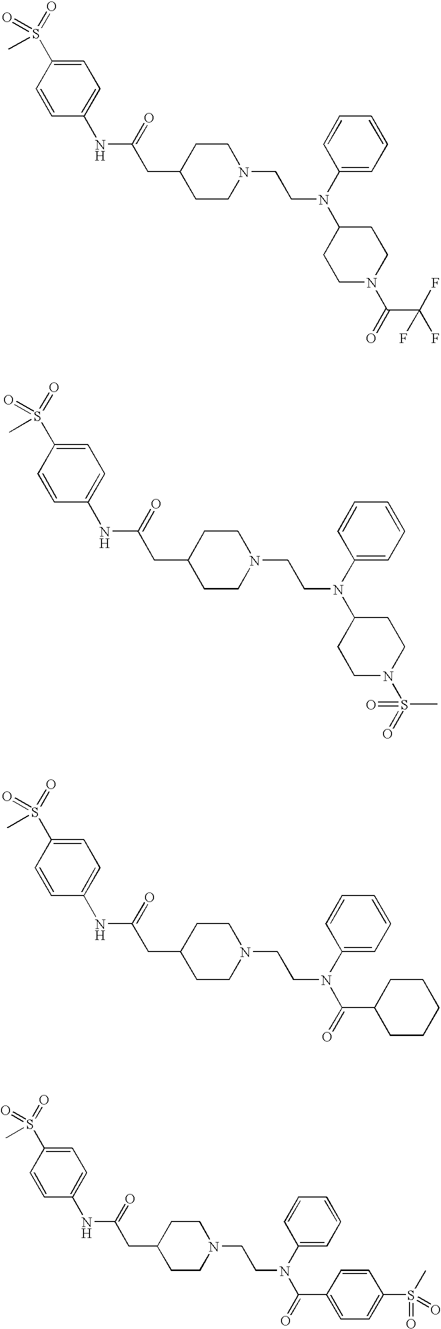 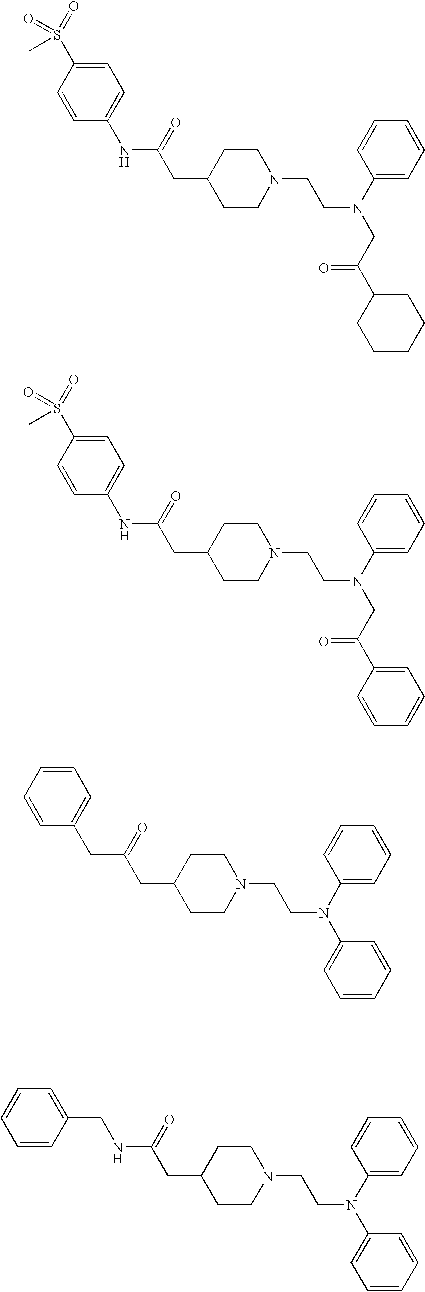 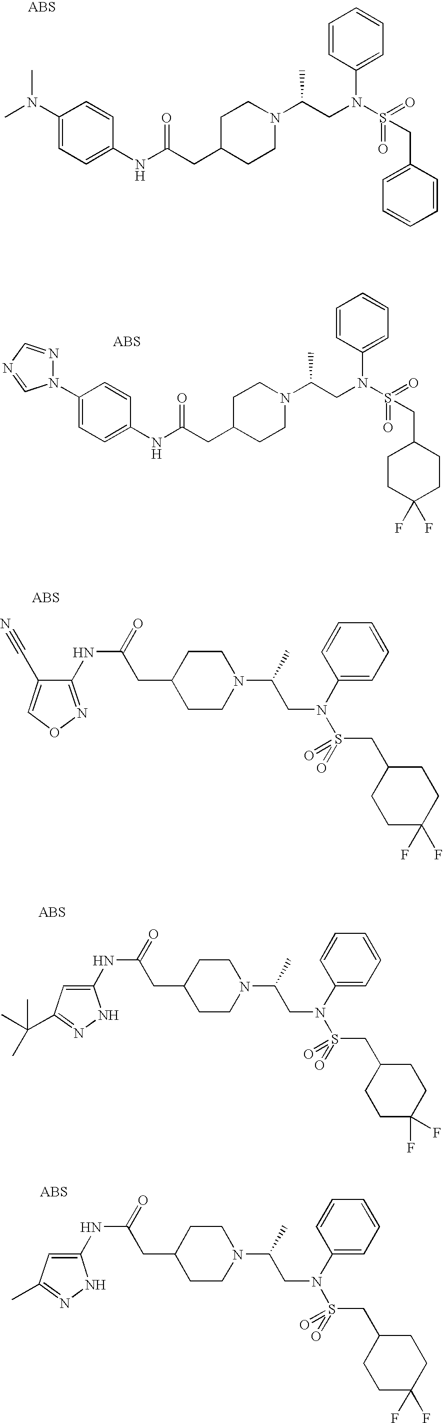 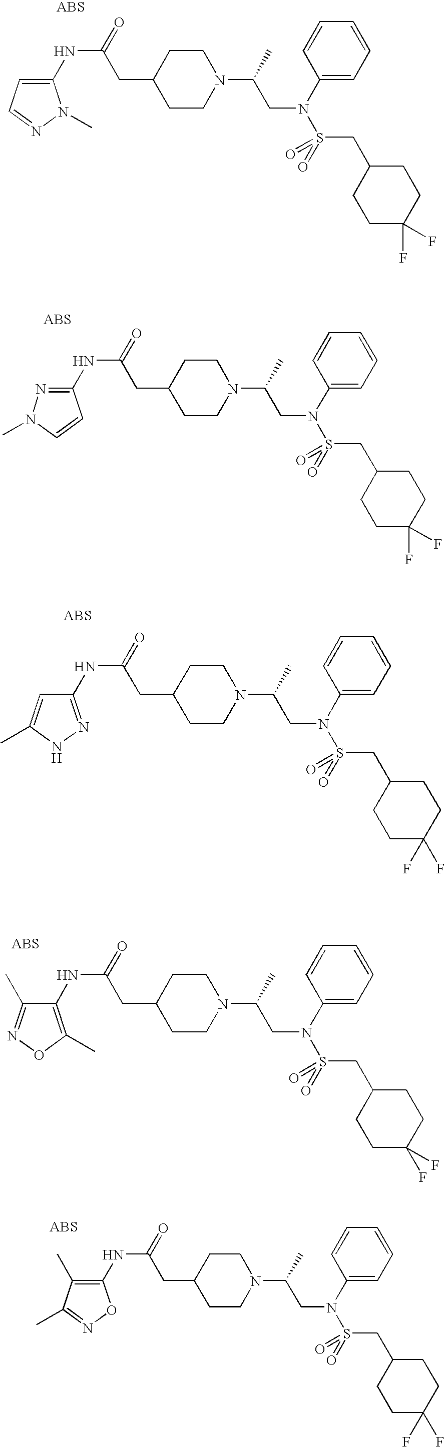 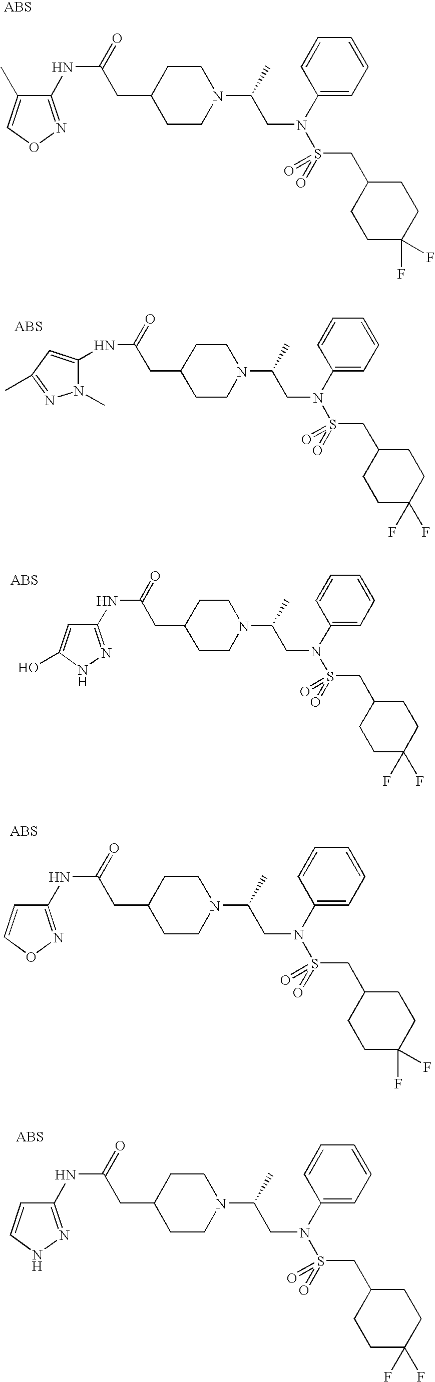 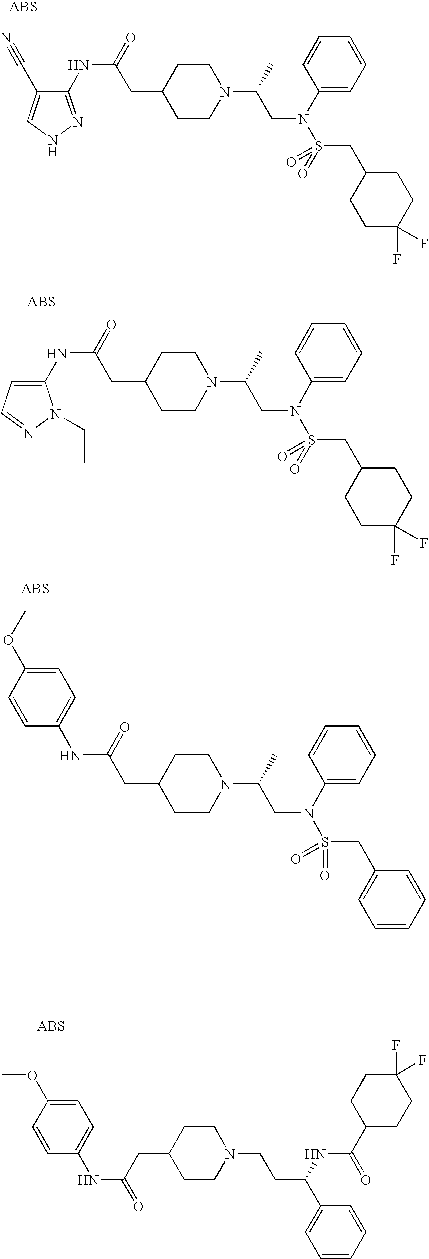 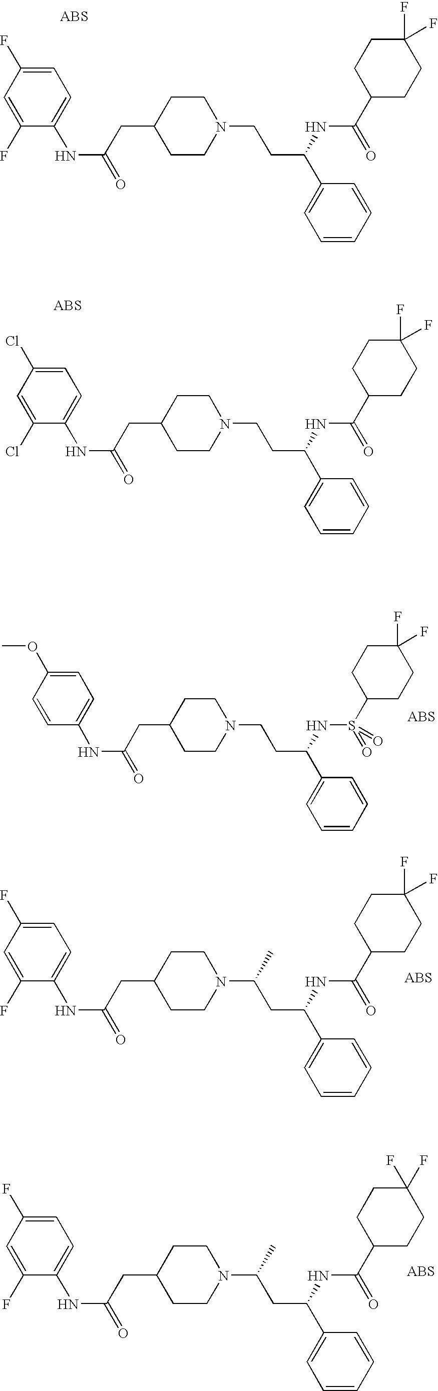 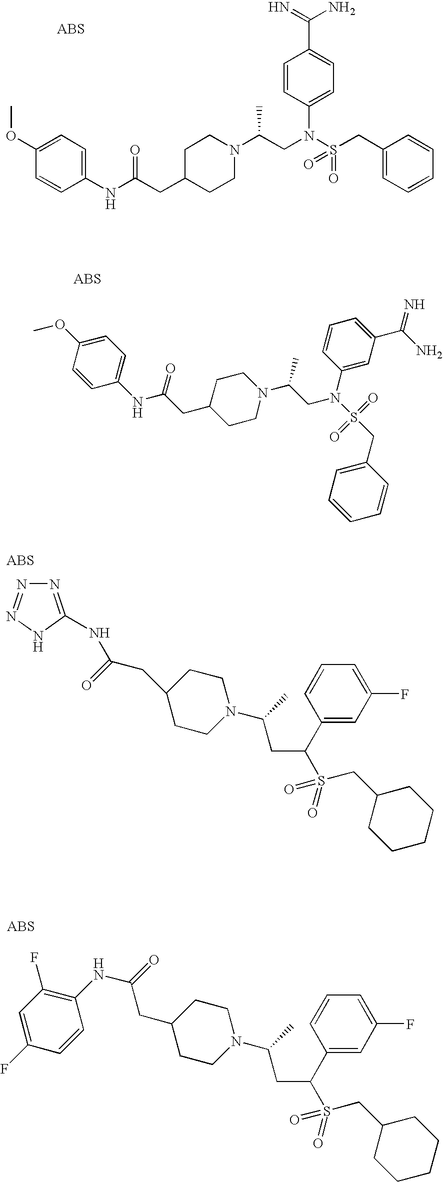 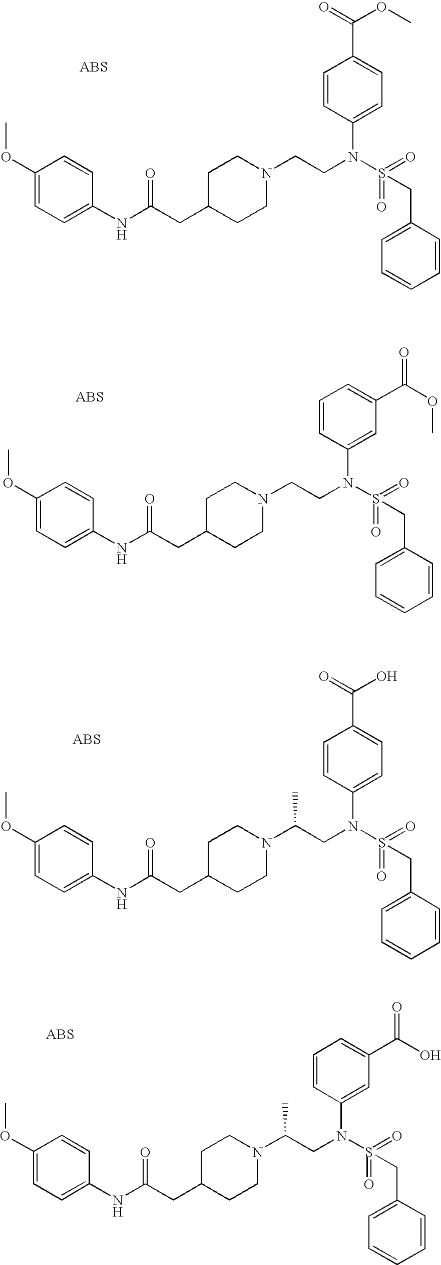 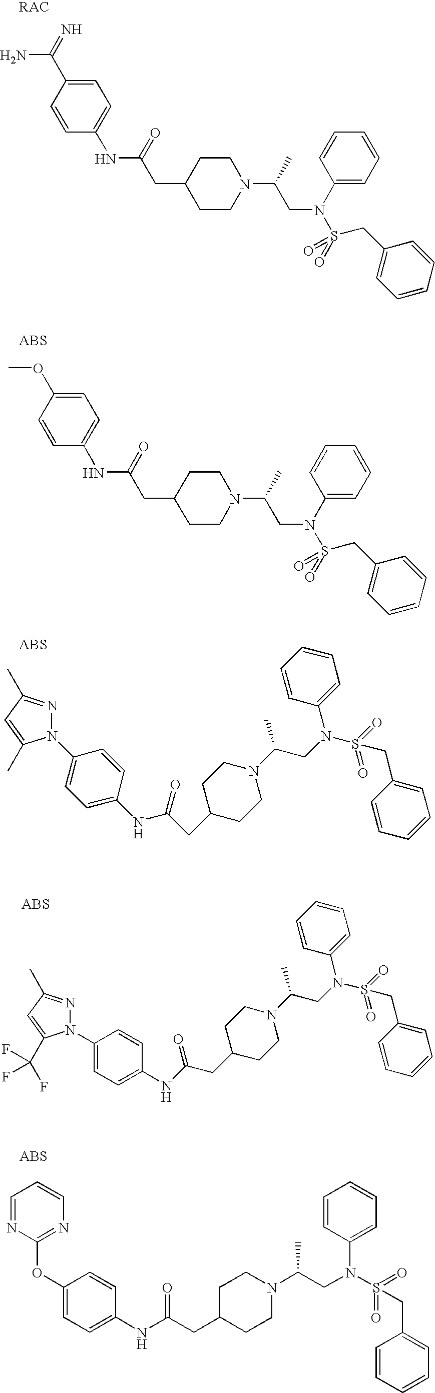 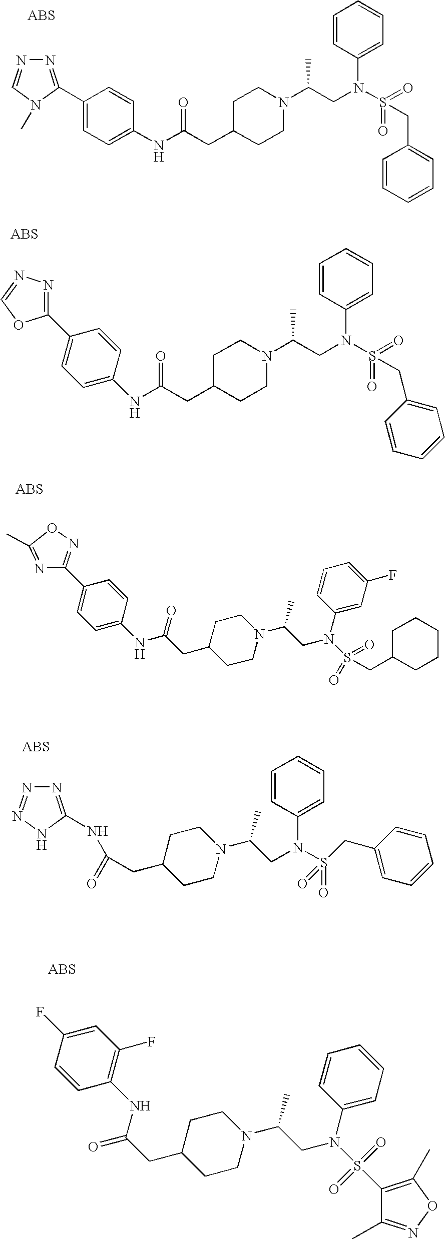 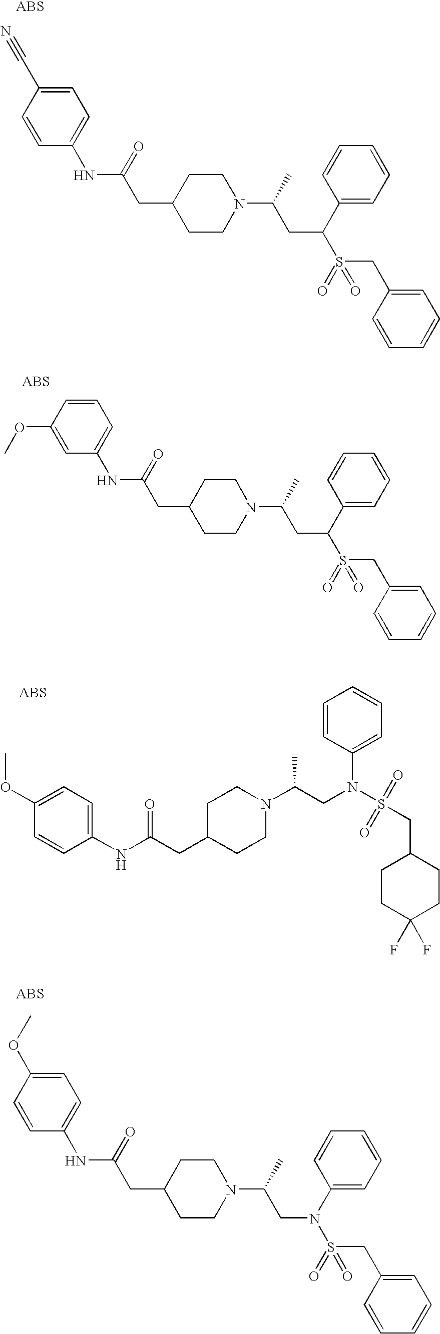 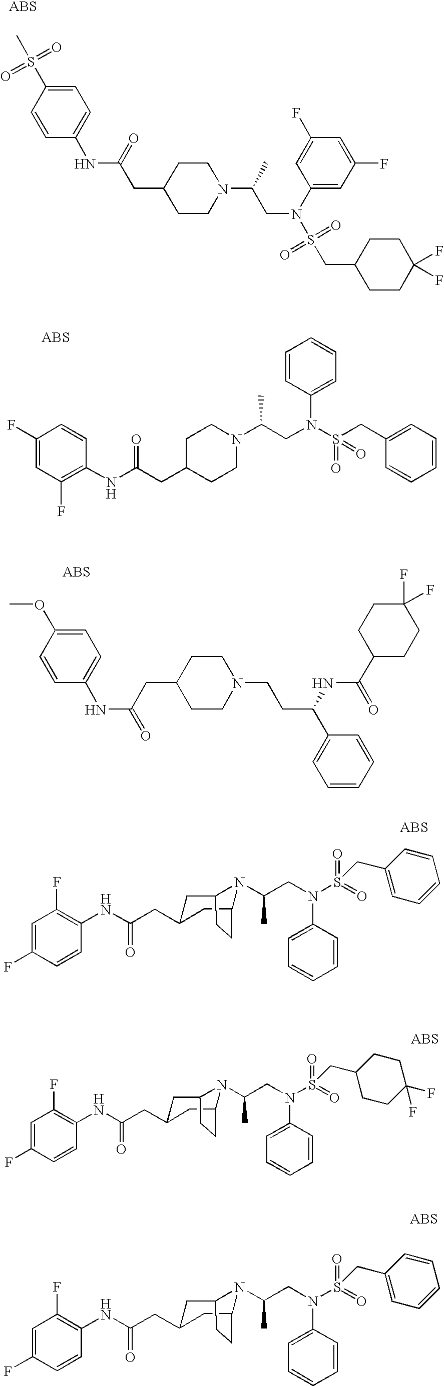
and pharmaceutically acceptable salts and solvates thereof.

13. A pharmaceutical composition comprising a compound according to claim 1 or a pharmaceutically acceptable salt or solvate thereof and at least one pharmaceutically acceptable carrier, diluent, excipient and/or adjuvant.

14-23. (canceled)

24. A method of treating and/or preventing autoimmune, inflammatory, infectious, proliferative or hyperproliferative diseases, or immunologically mediated diseases, comprising administering a therapeutically effective amount of a compound according to claim 1 or a pharmaceutically acceptable salt or solvate thereof or a pharmaceutical composition according to claim 13 to a patient in need thereof.

25. The method according to claim 24, wherein the disease is selected from AIDS, inflammatory and immunoregulatory disorders and diseases including asthma, pulmonary emphysema, allergic diseases and graft rejection as well as autoimmune pathologies such as rheumatoid arthritis, atherosclerosis, psoriasis, systemic lupus erythematosus, ulcerative colitis, multiple sclerosis, glomerulonephritis, together with chronic obstructive pulmonary disease (COPD, including pulmonary fibrosis), metastatic cancers and renal diseases.

26. The method according to claim 25, wherein the disease is AIDS caused by HIV-1 or -2 infection.

27. A method of inhibiting the entry of viruses into target cells and, therefore, for the prevention of infection by viruses, the treatment of infection by viruses, comprising administering a therapeutically effective amount of a compound according to claim 1 or a pharmaceutically acceptable salt or solvate thereof or a pharmaceutical composition according to claim 13 to a patient in need thereof.

28. The method according to claim 27, wherein the virus is human immunodeficiency virus.

29. The method according to claim 28 for the prevention and/or treatment of acquired immune deficiency syndrome (AIDS).

30. A method of modulating chemokine receptor activity in a patient comprising administering a therapeutically effective amount of a compound according to claim 1 or a pharmaceutically acceptable salt or solvate thereof or a pharmaceutical composition according to claim 13 to a patient in need thereof.

31. The method according to claim 30, wherein the chemokine is CCR5.

32. The method according to claim 24, wherein said compound or the pharmaceutically acceptable salt or solvate thereof or the pharmaceutical composition is administered in combination with at least one additional therapeutic agent and/or active ingredient.

33. The method according to claim 27, wherein said compound or the pharmaceutically acceptable salt or solvate thereof, or the pharmaceutical composition is administered in combination with at least one additional therapeutic agent and/or active ingredient.

34. The method according to claim 30, wherein said compound or the pharmaceutically acceptable salt or solvate thereof, or the pharmaceutical composition is administered in combination with at least one additional therapeutic agent and/or active ingredient.
